(19) United States
(12) Patent Application Publication
(10) Pub. No.: US 2025/0273295 A1
(43) Pub. Date: Aug. 28, 2025

(54) DETECTING THE PRESENCE OF A TUMOR BASED ON METHYLATION STATUS OF CELL-FREE NUCLEIC ACID MOLECULES

(71) Applicant: GUARDANT HEALTH, INC., Palo Alto, CA (US)

(72) Inventors: Yupeng HE, Alhambra, CA (US); Zhaoren HE, La Jolla, CA (US); Ariel JAIMOVICH, Redwood City, CA (US); William Walter YOUNG GREENWALD, San Diego, CA (US); William J. GREENLEAF, Menlo Park, CA (US); Sai CHEN, San Diego, CA (US); Tingting JIANG, San Diego, CA (US); Jun ZHAO, San Diego, CA (US)

(21) Appl. No.: 18/907,227

(22) Filed: Oct. 4, 2024

Related U.S. Application Data
(63) Continuation of application No. PCT/US2023/065560, filed on Apr. 7, 2023.
(60) Provisional application No. 63/336,852, filed on Apr. 29, 2022.
(60) Provisional application No. 63/328,602, filed on Apr. 7, 2022.

(51) Int. Cl. G16B 20/20; A61B 10/00; G16B 40/00; G16H 10/40; G16H 50/20

(52) U.S. Cl. CPC G16B 20/20; A61B 10/0041; G16B 40/00; G16H 10/40; G16H 50/20

(57) ABSTRACT

In implementations described herein, methylation information is determined with respect to classification regions of a reference genome that are related to the presence of a tumor in a subject. The methylation information can be analyzed using a number of computational techniques to provide metrics related to the presence or absence of a tumor in a given subject.

### CROSS REFERENCE TO RELATED APPLICATION

The present application is a Continuation of International Patent Application No. PCT/US2023/065560, filed Apr. 7, 2023, which claims the benefit of U.S. Provisional Application No. 63/328,602, filed Apr. 7, 2022, and U.S. Provisional Application No. 63/336,852, filed Apr. 29, 2022. All are incorporated by reference in their entirety for all purposes.

### BACKGROUND

Cancer is a major cause of disease worldwide. Each year, tens of millions of people are diagnosed with cancer around the world, and more than half eventually die from it. In many countries, cancer ranks the second most common cause of death following cardiovascular diseases. Early detection is associated with improved outcomes for many cancers.

Cancer can be caused by the accumulation of genetics variations within an individual's normal cells, at least some of which result in improperly regulated cell division. Such variations commonly include copy number variations (CNVs), single nucleotide variations (SNVs), gene fusions, insertions and/or deletions (indels), epigenetic variations including 5-methylation of cytosine (5-methylcytosine) and association of DNA with chromatin and transcription factors.

Cancers are often detected by biopsies of tumors followed by analysis of cells, markers or DNA extracted from cells. But more recently it has been proposed that cancers can also be detected from cell-free nucleic acids in body fluids, such as blood or urine. Such tests have the advantage that they are noninvasive and can be performed without identifying suspected cancer cells in biopsy. However, such tests are complicated by the fact that the amount of nucleic acids in body fluids is very low and what nucleic acids are present are heterogeneous in form (e.g., RNA and DNA, single-stranded and double-stranded, and various states of post-replication modification and association with proteins, such as histones).

Thus, there is a need for improved systems and methods for improved cancer detection using liquid biopsy assays. Therefore, it is an object of the disclosure to provide computer-implemented systems and methods that have improved capability to classify a sample as containing tumor-derived DNA with heightened sensitivity.

### BRIEF DESCRIPTION OF THE DRAWINGS

The accompanying drawings, which are incorporated in and constitute a part of this specification, illustrate certain implementations, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a diagrammatic representation of an example environment 100 that identifies nucleic acids that correspond to classification regions of a reference sequence, where the classification regions have at least a threshold number of CpGs

FIG. 2 is a diagrammatic representation of an example architecture to determine tumor metrics based on one or more models that analyze methylation status of cell free nucleic acid molecules, according to one or more implementations.

FIG. 3 is a diagrammatic representation of an example architecture to train one or more machine learning models to determine cancer metrics based on methylation status of cell-free nucleic acid molecules, according to one or more implementations.

FIG. 4 is a flow diagram of an example process to determine tumor metrics related to levels of methylation of classification regions of a reference sequence, according to one or more implementations.

FIG. 5 is a block diagram illustrating components of a machine, in the form of a computer system, that may read and execute instructions from one or more machine-readable media to perform any one or more methodologies described herein, in accordance with one or more example implementations.

FIG. 6 is block diagram illustrating a representative software architecture that may be used in conjunction with one or more hardware architectures described herein, in accordance with one or more example implementations.

FIGS. 7A, 7B, and 7C are graphical representations showing promoter-methylation calls in training and test samples among 88 TSG+HRD genes.

FIG. 8A is a graphical representation showing cancer-prediction scores in cancer-free samples that have >1 call. FIG. 8B is a table showing genes that called most often for promoter methylation in cancer-free donors. FIG. 8C is a table showing in-silico LoD estimates in selected genes from cell line KM12.

FIG. 9 is a graphical representation showing prevalence of promoter methylation in different types of cancer of TSG and HRD genes, our test dataset (total N=559), and in TCGA public data (total N=2,380). Limited to genes with promoter methylation in TCGA.

FIG. 10A is a graphical representation showing MLH1 promoter methylation in cancer-free donors and CRC patients (MSI-H and MSS). FIG. 10B is a table showing calls of MLH1 promoter methylation and BRAF-V600E in CRC patients.

FIG. 11 is a table showing an overview of the training and the test datasets for Example 5.

FIG. 12A is a graph graphical representation showing model performance for the prediction of CRC/cancer-free status in the training set. Shadows indicate variations in iterations. FIG. 12B is a table showing performance of cancer prediction models on the independent test dataset.

FIG. 13A is a table showing CV of TF estimates from genomic calls and methylation in the in-vitro dataset. FIG. 13B is a graphical representation showing TF model performance (black lines for diagonals) in the training set of CRC and cancer-free samples (cross-validation) FIG. 13C is a graphical representation showing the in-silico dataset for lower truth TFs.

FIG. 14 is a graphical representation showing distribution of predicted TF for CRC patients in the training set with and without driver mutations.

FIG. 15A is a graphical representation showing positivity rates in individual for lung cancer detection in stage I/II patients and in stage III/IV. patients.

FIG. 15B is a graphical representation showing positivity rates in individuals for multi-cancer detection (bladder, gastric, ovarian, pancreatic, and liver) in stage I/II patients and in stage III/IV patients.

FIG. 16 is a graphical representation showing positivity rates in individuals for multi-cancer detection (bladder, gastric, ovarian, pancreatic, and liver) in stage I patients, stage II patients, stage Ill patients, and stage IV patients.

FIG. 17 is a graphical representation of epigenomic MAF in relation to target MAF for colorectal cancer, lung cancer, and breast cancer.

FIG. 18 is a table showing that the quantitative precision of epigenomics cTF is capable of reaching an LoQ of less than 0.1% in CRC, lung and breast clinical samples.

FIG. 19A is a graphical representation showing that the somatic mutation based CTF is robust for replicates within the same cTF levels, particularly at cTF levels of 0.5% or higher.

FIG. 19B is a graphical representation showing that the epigenomic cTF can maintain a 100% evaluation rate and has a LoQ down to 0.1% cTF.

FIG. 20A is a graphical representation of methylation signals and somatic mutations for a first replicate of clinical titrations.

FIG. 20B is a graphical representation of methylation signals and somatic mutations for a second replicate of clinical titrations.

FIG. 21 is a table indicating ctDNA level changes for the first replicate and the second replicate calculated using a genomic-only method and a methylation method.

FIG. 22 is a graphical representation of epigenomic vs genomic cTF on clinical samples (one point for one sample).

FIG. 23 is a graphical representation of the epiMAF distribution in early and late-stage cancer patients for breast cancer, colorectal cancer, lung cancer, and a group of other cancers.

FIG. 24 is a graphical representation showing a probability distribution indicating the number of methylated cytosines included in the three partitions.

FIG. 25A includes a graphical representation showing changes to metrics for a first classification region for a first group of samples treated with MBD using a first set of reagents and a second group of samples treated with MBD using a second set of reagents.

FIG. 25B includes a graphical representation showing changes to metrics for a second classification region for a first classification region for a first group of samples treated with MBD using a first set of reagents and a second group of samples treated with MBD using a second set of reagents.

### SUMMARY

In one or more aspects, a method includes obtaining, by a computing system having one or more hardware processors and memory, training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The method also includes analyzing, by the computing system, the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The method also includes analyzing, by the computing system, the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The method also includes determining, by the computing system, a metric for the individual classification regions of the plurality of classification regions based on the first quantitative measure for the individual classification regions and the second quantitative measure for the plurality of control regions. The method also includes generating, by the computing device, training data that includes the metric for the individual classification regions of the plurality of classification regions for the training sequence reads from samples of training subjects. The method also includes implementing, by the computing system and using the training data, one or more machine learning algorithms to generate a model to determine an indication of cancer being present in subjects based on amounts of methylated cytosines in at least a portion of the plurality of classification regions, the model including weights for individual classification regions of the plurality of classification regions and at least a portion of the weights of the individual classification regions being different from one another.

In one or more aspects, the method includes obtaining, by the computing system, testing sequence data from an additional subject that is not included in the plurality of subjects, the testing sequence data including testing sequencing reads derived from a sample of the additional subject, individual testing sequencing reads including a nucleotide sequence corresponding to a fragment of a nucleic acid included in the additional sample and individual testing sequencing reads corresponding to molecules having at least the threshold amount of methylated cytosines included in regions of the nucleotide sequence having at least the threshold cytosine-guanine content; and determining, using the model and the additional sequence data, the indication of cancer being present in the additional subject.

In one or more aspects, the method includes analyzing, by the computing system, the testing sequencing reads to determine a first quantitative measure derived from the testing sequencing reads that correspond to the individual classification regions of the plurality of classification regions; analyzing, by the computing system, the testing sequencing reads to determine a second quantitative measure derived from the testing sequencing reads that correspond to the individual control regions the plurality of control regions; determining, by the computing system, the metric for the individual classification regions based on the first quantitative measure for the individual classification regions and the second quantitative measure for the plurality of control regions; and generating, by the computing system, an input vector that includes the metrics for the individual classification regions, where the model uses the input vector to determine the indication of cancer being present in the additional subject.

In one or more aspects, the one or more machine learning algorithms include one or more classification algorithms and the indication of cancer being present corresponds to a probability of cancer being present in the additional subject.

In one or more aspects, the one or more machine learning algorithms include one or more regression algorithms and the indicator corresponds to an estimate of tumor fraction of the additional sample.

In one or more aspects, the training sequencing reads comprise a first portion of the training sequence data and additional training sequencing reads comprise a second portion of the training sequence data, where the additional training sequencing reads are different from the training sequencing reads and the method includes analyzing, by the computing system, at least one of the first portion of the training sequence data or the second portion of the training sequence data to determine an individual frequency of a plurality of variants present in an individual sample of the plurality of samples; determining, by the computing system and for the individual samples, a variant of the plurality of variants having a maximum frequency that corresponds to the individual frequency having a greatest value among individual frequencies derived from an individual sample; and determining, by the computing system, individual measures of tumor fraction for an individual sample based on the greatest value of the individual frequencies derived from the individual sample.

In one or more aspects, the training data includes the individual measures of tumor fraction for the individual samples of the plurality of samples and the model is generated based on the individual measures of tumor fraction for the individual samples of the plurality of samples.

In one or more aspects, the metric for the individual classification regions is determined based on a scaling factor and an error correction factor.

In one or more aspects, the plurality of classification regions individually correspond to genomic regions in which a methylation rate of the genomic regions in nucleic acids derived from cells obtained from subjects in which cancer is present is different from a methylation rate of the genomic regions in nucleic acids derived from cells obtained from subjects in which cancer is not present.

In one or more aspects, the plurality of classification regions correspond to a first plurality of classification regions for a first cancer type and the model can be generated for a second cancer type based on a second plurality of classification regions that are different from the first plurality of classification regions.

In one or more aspects, the plurality of samples and the additional sample include cell free nucleic acids.

In one or more aspects, the method includes performing, by the computing system, a training process using the training data to generate the model, where the training process includes determining, by the computing system, one or more additional weights of individual samples included in the training data based on the indication of cancer for the individual samples being within a threshold confidence level.

In one or more aspects, the indication of cancer for an individual sample is outside of the threshold confidence level and the method includes applying, by the computing system, a penalty to a weight of the individual sample during the training process.

In one or more aspects, the method includes performing, by the computing system and using the one or more machine learning algorithms, one or more first iterations of the training process for the model using a portion of the training data; and generating, by the computing system, first output data for the model based on the one or more first iterations of the training process, the first output data corresponding to one or more first additional indications of cancer being present in first individual subjects of the plurality of subjects, the first individual subjects corresponding to the portion of the training data.

In one or more aspects, the method includes combining, by the computing system, the first output data and the training data to produce additional training data; performing, by the computing system, one or more second iterations of the training process for the model using a portion of the additional training data; and generating, by the computing system, second output data for the model based on the one or more second iterations of the training process, the second output data indicating one or more second additional indications of cancer being present in second individual subjects of the plurality of subjects, the second individual subjects corresponding to the portion of the additional training data.

In one or more aspects, the weights for the individual classification regions of the plurality of classification regions are determined based on the first output data and the second output data.

In one or more aspects, the method includes determining, by the computing system, that a number of indications of cancer being present that were determined during one or more iterations of the training process are at least a threshold value for one or more samples included in the training data; and determining, by the computing system, that modifications to one or more weights of the model are not modified or are modified by a minimal amount.

In one or more aspects, the method includes determining, by the computing system, that an additional number of indications of cancer being present that were determined during the one or more iterations of the training process are less than the threshold value for one or more additional samples included in the training data; and determining, by the computing system, that modifications to one or more additional weights of the model are modified by more than the minimal amount.

In one or more aspects, the method includes combining a plurality of nucleic acids derived from at least one of blood or tissue of a subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content.

In one or more aspects, a wash of the plurality of washes is performed with a solution having a concentration of sodium chloride (NaCl) and produces a nucleic acid fraction of the number of nucleic acid fractions having a range of binding strengths to MBD proteins.

In one or more aspects, the method includes determining that a first nucleic acid fraction is associated with a first partition of a plurality of partitions of nucleic acids, the first partition corresponding to a first range of binding strengths to MBD proteins; attaching a first molecular barcode to nucleic acids of the first nucleic acid fraction, the first molecular barcode being included in a first set of molecular barcodes associated with the first partition; determining that a second nucleic acid fraction is associated with a second partition of the plurality of partitions of nucleic acids, the second partition corresponding to a second range of binding strengths to MBD proteins different from the first range of binding strengths to MBD proteins; and attaching a second molecular barcode to nucleic acids of the second nucleic acid fraction, the second molecular barcode being included in a second set of molecular barcodes associated with the second partition.

In one or more aspects, the method includes combining at least a portion of the number of nucleic acid fractions with an amount of one or more methylation sensitive restriction enzymes that cleave molecules with one or more unmethylated cytosines to produce at least a portion of the plurality of samples used to produce the sequencing reads.

In one or more aspects, the method includes combining at least a portion of the number of nucleic acid fractions with an amount of one or more methylation dependent restriction enzymes that cleaves molecules with one or more methylated cytosines to produce at least a portion of the plurality of samples used to produce the sequencing reads.

In one or more aspects, a limit of detection for the model to determine tumor fraction of samples is no greater than 0.05%.

In one or more aspects, a computing system includes: one or more hardware processors; and one or more non-transitory computer-readable storage media including computer-readable instructions that, when executed by the one or more hardware processors, cause the one or more hardware processors to perform operations comprising: obtaining training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The operations also include analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The operations also include analyzing the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The operations also include determining a metric for the individual classification regions of the plurality of classification regions based on the first quantitative measure for the individual classification regions and the second quantitative measure for the plurality of control regions. The operations also include generating training data that includes the metric for the individual classification regions of the plurality of classification regions for the training sequence reads from samples of training subjects. The operations also include implementing, using the training data, one or more machine learning algorithms to generate a model to determine an indication of cancer being present in subjects based on amounts of methylated cytosines in at least a portion of the plurality of classification regions, the model including weights for individual classification regions of the plurality of classification regions and at least a portion of the weights of the individual classification regions being different from one another.

In one or more aspects, the computing system may obtain testing sequence data from an additional subject that is not included in the plurality of subjects. The testing sequence data may include testing sequencing reads derived from a sample of the additional subject with individual testing sequencing reads including a nucleotide sequence corresponding to a fragment of a nucleic acid included in the additional sample and individual testing sequencing reads corresponding to molecules having at least the threshold amount of methylated cytosines included in regions of the nucleotide sequence having at least the threshold cytosine-guanine content. The operations may also include determining, using the model and the additional sequence data, the indication of cancer being present in the additional subject.

In one or more aspects, the computing system may analyze the testing sequencing reads to determine a first quantitative measure derived from the testing sequencing reads that correspond to the individual classification regions of the plurality of classification regions. The computing system may also analyze the testing sequencing reads to determine a second quantitative measure derived from the testing sequencing reads that correspond to the individual control regions the plurality of control regions. The computing system may also determine the metric for the individual classification regions based on the first quantitative measure for the individual classification regions and the second quantitative measure for the plurality of control regions. The computing system may also generate an input vector that includes the metrics for the individual classification regions, where the model uses the input vector to determine the indication of cancer being present in the additional subject.

In one or more aspects of the computing system, the one or more machine learning algorithms include one or more classification algorithms and the indication of cancer being present corresponds to a probability of cancer being present in the additional subject.

In one or more aspects of the computing system, the one or more machine learning algorithms include one or more regression algorithms and the indicator corresponds to an estimate of tumor fraction of the additional sample.

In one or more aspects of the computing system, the training sequencing reads comprise a first portion of the training sequence data and additional training sequencing reads comprise a second portion of the training sequence data, where the additional training sequencing reads are different from the training sequencing reads and the computing system may analyze at least one of the first portion of the training sequence data or the second portion of the training sequence data to determine an individual frequency of a plurality of variants present in an individual sample of the plurality of samples. The computing system may also determine, for the individual samples, a variant of the plurality of variants having a maximum frequency that corresponds to the individual frequency having a greatest value among individual frequencies derived from an individual sample. The computing system may also determine individual measures of tumor fraction for an individual sample based on the greatest value of the individual frequencies derived from the individual sample.

In one or more aspects of the computing system, the training data includes the individual measures of tumor fraction for the individual samples of the plurality of samples and the model is generated based on the individual measures of tumor fraction for the individual samples of the plurality of samples.

In one or more aspects of the computing system, the metric for the individual classification regions is determined based on a scaling factor and an error correction factor.

In one or more aspects of the computing system, the plurality of classification regions individually correspond to genomic regions in which a methylation rate of the genomic regions in nucleic acids derived from cells obtained from subjects in which cancer is present is different from a methylation rate of the genomic regions in nucleic acids derived from cells obtained from subjects in which cancer is not present.

In one or more aspects of the computing system, the plurality of classification regions correspond to a first plurality of classification regions for a first cancer type and the model can be generated for a second cancer type based on a second plurality of classification regions that are different from the first plurality of classification regions.

In one or more aspects, the computing system may perform a training process using the training data to generate the model, where the training process includes determining one or more additional weights of individual samples included in the training data based on the indication of cancer for the individual samples being within a threshold confidence level.

In one or more aspects of the computing system, the indication of cancer for an individual sample is outside of the threshold confidence level and the method includes applying, by the computing system, a penalty to a weight of the individual sample during the training process.

In one or more aspects, the computing system may perform, using the one or more machine learning algorithms, one or more first iterations of the training process for the model using a portion of the training data. The computing system may also generate first output data for the model based on the one or more first iterations of the training process, the first output data corresponding to one or more first additional indications of cancer being present in first individual subjects of the plurality of subjects, where the first individual subjects corresponding to the portion of the training data.

In one or more aspects, the computing system may combine the first output data and the training data to produce additional training data; perform one or more second iterations of the training process for the model using a portion of the additional training data; and generate second output data for the model based on the one or more second iterations of the training process, with the second output data indicating one or more second additional indications of cancer being present in second individual subjects of the plurality of subjects, and the second individual subjects corresponding to the portion of the additional training data.

In one or more aspects of the computing system, the weights for the individual classification regions of the plurality of classification regions are determined based on the first output data and the second output data.

In one or more aspects, the computing system may determine that a number of indications of cancer being present that were determined during one or more iterations of the training process are at least a threshold value for one or more samples included in the training data. The computing system may also determine that modifications to one or more weights of the model are not modified or are modified by a minimal amount.

In one or more aspects, the computing system may determine that an additional number of indications of cancer being present that were determined during the one or more iterations of the training process are less than the threshold value for one or more additional samples included in the training data; and determine that modifications to one or more additional weights of the model are modified by more than the minimal amount.

In one or more aspects of the computing system, a limit of detection for the model to determine tumor fraction of samples is no greater than 0.05%.

In one or more aspects, one or more computer-readable storage media comprise computer-readable instructions that, when executed by one or more processors of a computing system, cause the computing system to perform operations comprising: obtaining training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The operations also include analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The operations also include analyzing the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The operations also include determining a metric for the individual classification regions of the plurality of classification regions based on the first quantitative measure for the individual classification regions and the second quantitative measure for the plurality of control regions. The operations also include generating training data that includes the metric for the individual classification regions of the plurality of classification regions for the training sequence reads from samples of training subjects. The operations also include implementing, using the training data, one or more machine learning algorithms to generate a model to determine an indication of cancer being present in subjects based on amounts of methylated cytosines in at least a portion of the plurality of classification regions, the model including weights for individual classification regions of the plurality of classification regions and at least a portion of the weights of the individual classification regions being different from one another.

In one or more aspects, a method includes obtaining, by a computing system having one or more hardware processors and memory, sequencing reads derived from a sample obtained from a subject, where individual sequencing reads include a nucleotide sequence corresponding to a fragment of a nucleic acid included in the sample and correspond to molecules having a threshold amount of methylated cytosines included in regions of the nucleotide sequence having at least a threshold cytosine-guanine content. The method may also include determining, by the computing system, a first quantitative measure derived from the sequencing reads that corresponds to individual classification regions of a plurality of classification regions, where at least a portion of the individual classification regions of the plurality of classification regions correspond to genomic regions of a reference genome that have the threshold amount of methylated cytosines in subjects in which cancer is detected and that have at least the threshold cytosine-guanine content. The method may also include analyzing, by the computing system, the sequencing reads to determine a second quantitative measure derived from the sequencing reads that correspond to a plurality of control regions, where individual control regions of the plurality of control regions correspond to additional genomic regions of the reference genome that have at least the threshold cytosine-guanine content and that have at least the threshold amount of methylated cytosines in subjects in which cancer is detected and in additional subjects in which cancer is not detected. The method may also include determining, by the computing system, a plurality of metrics with individual metrics of the plurality of metrics corresponding to individual classification regions of the plurality of classification regions based on the first quantitative measure for the individual classification regions and the second quantitative measure for the plurality of control regions. The method may also include determining, by the computing system, an indication of cancer being present in the subject based on at least a portion of the plurality of metrics.

In one or more aspects, the method may also include includes determining, by the computing system and using the sequencing data, a distribution of sequence representations for a differentially methylated region and determining, by the computing system, that at least a threshold amount of the sequence representations included in the distribution overlap with a subregion of the differentially methylated region. The method may also include determining, by the computing system, that the subregion of the differentially methylated region is a classification region of the plurality of classification regions.

In one or more aspects, the method may also include determining, by the computing system, an order of the values of the plurality of metrics, and determining, by the computing system, a subset of classification regions from among the plurality of classification regions based on the order, where a portion of the plurality of metrics that correspond to the subset of the classification regions is used to determine the indication of cancer being present in the subject.

In one or more aspects, the indication of cancer being present in the subject is an initial indication of cancer being present in the subject, and the method may also include applying, by the computing system, a scaling factor to the initial indication of cancer being present in the subject to determine a modified indication of cancer being present in the subject.

In one or more aspects, the indication of cancer being present in the subject corresponds to a tumor fraction.

In one or more aspects, the sample is a first sample collected at least one of before or at onset for treatment of cancer, and the method may also include obtaining, by the computing system having one or more hardware processors and memory, additional sequencing reads derived from a second sample obtained from the subject, where individual additional sequencing reads including an additional nucleotide sequence correspond to a fragment of a nucleic acid included in the second sample and correspond to additional molecules having the threshold amount of methylated cytosines included in regions of the additional nucleotide sequence having at least the threshold cytosine-guanine content. The method may also include determining, by the computing system, an additional first quantitative measure derived from the additional sequencing reads that corresponds to the individual classification regions of the plurality of classification regions, with at least a portion of the individual classification regions of the plurality of classification regions corresponding to genomic regions of the reference genome that have the threshold amount of methylated cytosines in subjects in which cancer is detected and that have at least the threshold cytosine-guanine content. The method may also include analyzing, by the computing system, the additional sequencing reads to determine an additional second quantitative measure derived from the additional sequencing reads that correspond to a plurality of control regions, with individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The method may also include determining, by the computing system, a plurality of additional metrics with individual additional metrics of the plurality of additional metrics corresponding to individual classification regions of the plurality of classification regions based on the additional first quantitative measure for the individual classification regions and the additional second quantitative measure for the plurality of control regions. The method may also include determining, by the computing system, an additional indication of cancer being present in the subject based on at least a portion of the plurality of additional metrics.

In one or more aspects, a method includes obtaining, by a computing system having one or more hardware processors and memory, testing sequence data from a subject, with the testing sequence data including testing sequencing reads derived from a sample of the subject, individual testing sequencing reads including a nucleotide sequence corresponding to a fragment of a nucleic acid included in the additional sample and individual testing sequencing reads corresponding to molecules having a threshold amount of methylated cytosines included in regions of the nucleotide sequence having at least the threshold cytosine-guanine content. The method may also include analyzing, by the computing system, the testing sequencing reads to determine a first quantitative measure derived from the testing sequencing reads that correspond to individual classification regions of a plurality of classification regions at least a portion of the individual classification regions of the plurality of classification regions corresponding to genomic regions of a reference genome that have the threshold amount of methylated cytosines in subjects in which cancer is detected and that have at least the threshold cytosine-guanine content. The method may also include analyzing, by the computing system, the testing sequencing reads to determine a second quantitative measure derived from the testing sequencing reads that correspond to individual control regions a plurality of control regions, with individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The method may also include determining, by the computing system, a metric for the individual classification regions based on the first quantitative measure for the individual classification regions and the second quantitative measure for the plurality of control regions. The method may also include generating, by the computing system, an input vector that includes the metrics for the individual classification regions. The method also includes determining, by the computing system, an indication of cancer being present in the subject by providing the input vector to a model that implements one or more machine learning techniques to generate indications of cancer being present in subjects, with the model including weights for individual classification regions of the plurality of classification regions and at least a portion of the weights of the individual classification regions being different from one another.

In one or more aspects, the method may also include includes obtaining, by the computing system having one or more hardware processors and memory, training sequence data including training sequencing reads derived from a plurality of samples of a plurality of training subjects, with individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The method may also include analyzing, by the computing system, the training sequencing reads to determine an additional first quantitative measure derived from the training sequencing reads that corresponds to individual classification regions of the plurality of classification regions. The method may also include analyzing, by the computing system, the training sequencing reads to determine an additional second quantitative measure derived from the training sequencing reads that correspond to a plurality of control regions. The method may also include determining, by the computing system, an additional metric for the individual classification regions of the plurality of classification regions based on the additional first quantitative measure for the individual classification regions and the additional second quantitative measure for the plurality of control regions. The method may also include generating, by the computing device, training data that includes the additional metric for the individual classification regions of the plurality of classification regions for the training sequence reads from samples of the plurality of training subjects. The method may also include implementing, by the computing system and using the training data, one or more machine learning algorithms to generate the model to determine the indications of cancer being present in subjects based on amounts of methylated cytosines in at least a portion of the plurality of classification regions.

In one or more aspects, the one or more machine learning algorithms include one or more classification algorithms, and the indication of cancer being present corresponds to a probability of cancer being present in the subject.

In one or more aspects, the one or more machine learning algorithms include one or more regression algorithms; and the indication corresponds to an estimate of tumor fraction of the sample.

In one or more aspects, the sample of the subject and the plurality of samples of the plurality of training subjects include cell free nucleic acids.

In one or more aspects, the threshold amount of sequence representations is at least about 70% of the sequence representations included in the distribution.

In one or more aspects, the second sample is obtained at least one week after the treatment of cancer is administered to the subject.

In one or more aspects, the method may also include analyzing, by the computing system, the indication of cancer being present in the subject in relation to the additional indication of cancer being present in the subject to determine a response to the treatment for the subject.

In one or more aspects, the training sequencing reads comprise a first portion of the training sequence data and additional training sequencing reads comprise a second portion of the training sequence data, where the additional training sequencing reads are different from the training sequencing reads; and the method may also include analyzing, by the computing system, at least one of the first portion of the training sequence data or the second portion of the training sequence data to determine an individual frequency of a plurality of variants present in an individual sample of the plurality of samples, and determining, by the computing system and for the individual sample, a variant of the plurality of variants having a maximum frequency that corresponds to the individual frequency having a greatest value among individual frequencies derived from an individual sample. The method may also include determining, by the computing system, individual measures of tumor fraction for an individual sample based on the greatest value of the individual frequencies derived from the individual sample.

In one or more aspects, the training data includes the individual measures of tumor fraction for the individual samples of the plurality of samples, and the model is generated based on the individual measures of tumor fraction for the individual samples of the plurality of samples.

In one or more aspects, a computing system comprises: a processor; and a memory storing instructions that, when executed by the processor, configure the computing system to: obtain sequencing reads derived from a sample obtained from a subject, individual sequencing reads including a nucleotide sequence corresponding to a fragment of a nucleic acid included in the sample and corresponding to molecules having a threshold amount of methylated cytosines included in regions of the nucleotide sequence having at least a threshold cytosine-guanine content. The computing system may also determine a first quantitative measure derived from the sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The computing system may also analyze the sequencing reads to determine a second quantitative measure derived from the sequencing reads that correspond to a plurality of control regions, with individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The computing system may also determine a plurality of metrics with individual metrics of the plurality of metrics corresponding to individual classification regions of the plurality of classification regions based on the first quantitative measure for the individual classification regions and the second quantitative measure for the plurality of control regions. The computing system may also determine an indication of cancer being present in the subject based on at least a portion of the plurality of metrics.

In one or more aspects of the computing system, the threshold amount of sequence representations is at least about 70% of the sequence representations included in the distribution.

In one or more aspects, the computing system may determine, using the sequencing data, a distribution of sequence representations for a differentially methylated region. The computing system may also determine that at least a threshold amount of the sequence representations included in the distribution overlap with a subregion of the differentially methylated region. The computing system may also determine, by the computing system, that the subregion of the differentially methylated region is a classification region of the plurality of classification regions.

In one or more aspects, the computing system may determine an order of the values of the plurality of metrics; and determine a subset of classification regions from among the plurality of classification regions based on the order; where a portion of the plurality of metrics that correspond to the subset of the classification regions is used to determine the indication of cancer being present in the subject.

In one or more aspects of the computing system, the indication of cancer being present in the subject is an initial indication of cancer being present in the subject, and the computing system may apply a scaling factor to the initial indication of cancer being present in the subject to determine a modified indication of cancer being present in the subject.

In one or more aspects of the computing system, the indication of cancer being present in the subject corresponds to a tumor fraction.

In one or more aspects of the computing system, the sample is a first sample collected at least one of before or at onset for treatment of cancer, and the computing system may obtain additional sequencing reads derived from a second sample obtained from the subject, with individual additional sequencing reads including an additional nucleotide sequence corresponding to a fragment of a nucleic acid included in the second sample and corresponding to additional molecules having the threshold amount of methylated cytosines included in regions of the additional nucleotide sequence having at least the threshold cytosine-guanine content. The computing system may also determine an additional first quantitative measure derived from the additional sequencing reads that corresponds to the individual classification regions of the plurality of classification regions, with at least a portion of the individual classification regions of the plurality of classification regions corresponding to genomic regions of the reference genome that have the threshold amount of methylated cytosines in subjects in which cancer is detected and that have at least the threshold cytosine-guanine content. The computing system may also analyze the additional sequencing reads to determine an additional second quantitative measure derived from the additional sequencing reads that correspond to a plurality of control regions, with individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The computing system may also determine a plurality of additional metrics with individual additional metrics of the plurality of additional metrics corresponding to individual classification regions of the plurality of classification regions based on the additional first quantitative measure for the individual classification regions and the additional second quantitative measure for the plurality of control regions. The computing system may also determine an additional indication of cancer being present in the subject based on at least a portion of the plurality of additional metrics.

In one or more aspects of the computing system, the second sample is obtained at least one week after the treatment of cancer is administered to the subject.

In one or more aspects, the computing system may analyze the indication of cancer being present in the subject in relation to the additional indication of cancer being present in the subject to determine a response to the treatment for the subject.

In one or more aspects, computing system comprising: a processor; and a memory storing instructions that, when executed by the processor, configure the system to: obtain testing sequence data from a subject, the testing sequence data including testing sequencing reads derived from a sample of the subject, individual testing sequencing reads including a nucleotide sequence corresponding to a fragment of a nucleic acid included in the additional sample and individual testing sequencing reads corresponding to molecules having a threshold amount of methylated cytosines included in regions of the nucleotide sequence having at least the threshold cytosine-guanine content. The computing system may also analyze the testing sequencing reads to determine a first quantitative measure derived from the testing sequencing reads that correspond to individual classification regions of a plurality of classification regions at least a portion of the individual classification regions of the plurality of classification regions corresponding to genomic regions of a reference genome that have the threshold amount of methylated cytosines in subjects in which cancer is detected and that have at least the threshold cytosine-guanine content. The computing system may also analyze the testing sequencing reads to determine a second quantitative measure derived from the testing sequencing reads that correspond to individual control regions a plurality of control regions, with individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The computing system may also determine a metric for the individual classification regions based on the first quantitative measure for the individual classification regions and the second quantitative measure for the plurality of control regions. The computing system may also generate an input vector that includes the metrics for the individual classification regions. The computing system may also determine an indication of cancer being present in the subject by providing the input vector to a model that implements one or more machine learning techniques to generate indications of cancer being present in subjects, with the model including weights for individual classification regions of the plurality of classification regions and at least a portion of the weights of the individual classification regions being different from one another.

In one or more aspects, the computing system may obtain training sequence data including training sequencing reads derived from a plurality of samples of a plurality of training subjects, with individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The computing system may also analyze the training sequencing reads to determine an additional first quantitative measure derived from the training sequencing reads that corresponds to individual classification regions of the plurality of classification regions. The computing system may also analyze the training sequencing reads to determine an additional second quantitative measure derived from the training sequencing reads that correspond to a plurality of control regions. The computing system may also determine an additional metric for the individual classification regions of the plurality of classification regions based on the additional first quantitative measure for the individual classification regions and the additional second quantitative measure for the plurality of control regions. The computing system may also generate training data that includes the additional metric for the individual classification regions of the plurality of classification regions for the training sequence reads from samples of the plurality of training subjects. The computing system may also implement, using the training data, one or more machine learning algorithms to generate the model to determine the indications of cancer being present in subjects based on amounts of methylated cytosines in at least a portion of the plurality of classification regions.

In one or more aspects of the computing system, the one or more machine learning algorithms include one or more classification algorithms; and the indication of cancer being present corresponds to a probability of cancer being present in the subject.

In one or more aspects of the computing system, the one or more machine learning algorithms include one or more regression algorithms; and the indication corresponds to an estimate of tumor fraction of the sample.

In one or more aspects of the computing system, the training sequence reads comprise a first portion of the training sequence data and additional training sequencing reads comprise a second portion of the training sequence data, where the additional training sequencing reads are different from the training sequencing reads and the computing system may: analyze at least one of the first portion of the training sequence data or the second portion of the training sequence data to determine an individual frequency of a plurality of variants present in an individual sample of the plurality of samples. The computing system may also determine, for the individual sample, a variant of the plurality of variants having a maximum frequency that corresponds to the individual frequency having a greatest value among individual frequencies derived from an individual sample. The computing system may also determine individual measures of tumor fraction for an individual sample based on the greatest value of the individual frequencies derived from the individual sample.

In one or more aspects of the computing apparatus, the training data includes the individual measures of tumor fraction for the individual samples of the plurality of samples and the model is generated based on the individual measures of tumor fraction for the individual samples of the plurality of samples.

In one or more aspects, a method includes obtaining, by a computing system having one or more hardware processors and memory, sequencing data from a plurality of subjects, the sequencing data including sequencing reads derived from a plurality of samples of the plurality of subjects, individual sequencing reads including a nucleotide sequence corresponding to a fragment of a nucleic acid included in the additional sample and individual sequencing reads corresponding to molecules having a threshold amount of methylated cytosines included in a promoter region of the nucleotide sequence having at least the threshold cytosine-guanine content. The method may also include analyzing, by the computing system, the sequencing reads to determine a first quantitative measure derived from the sequencing reads that corresponds to the promoter region. The method may also include analyzing, by the computing system, the sequencing reads to determine a second quantitative measure derived from the sequencing reads that correspond to individual control regions a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The method may also include determining, by the computing system, a metric for the promoter region based on the first quantitative measure for the individual classification regions and the second quantitative measure for the plurality of control regions. The method may also include generating, by the computing system, an indication of methylation status of the promoter region based on the metric having at least a threshold value.

In one or more aspects, a computing system may include one or more hardware processors; and one or more non-transitory computer-readable storage media including computer-readable instructions that, when executed by the one or more hardware processors, cause the one or more hardware processors to perform operations comprising: obtaining sequencing data from a plurality of subjects, the sequencing data including sequencing reads derived from a plurality of samples of the plurality of subjects, individual sequencing reads including a nucleotide sequence corresponding to a fragment of a nucleic acid included in the additional sample and individual sequencing reads corresponding to molecules having a threshold amount of methylated cytosines included in a promoter region of the nucleotide sequence having at least the threshold cytosine-guanine content. The system may also analyze the sequencing reads to determine a first quantitative measure derived from the sequencing reads that corresponds to the promoter region. The system may also analyze the sequencing reads to determine a second quantitative measure derived from the sequencing reads that correspond to individual control regions a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The system may also analyzing the sequencing reads to determine a second quantitative measure derived from the sequencing reads that correspond to individual control regions a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The system may also determine a metric for the promoter region based on the first quantitative measure for the individual classification regions and the second quantitative measure for the plurality of control regions. The system may also generate an indication of methylation status of the promoter region based on the metric having at least a threshold value.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth.

It is also to be understood that the terminology used herein is for the purpose of describing particular implementations only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About. As used herein, “about” or “approximately” as applied to one or more values or elements of interest, refers to a value or element that is similar to a stated reference value or element. In certain implementations, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Adapter. As used herein, “adapter” refers to a short nucleic acid (e.g., less than about 500 nucleotides, less than about 100 nucleotides, or less than about 50 nucleotides in length) that can be at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next-generation sequencing (NGS) applications. Adapters can also include binding sites for capture probes, such as an oligonucleotide attached to a flow cell support or the like. Adapters can also include a nucleic acid tag as described herein. Nucleic acid tags can be positioned relative to amplification primer and sequencing primer binding sites, such that a nucleic acid tag is included in amplicons and sequence reads of a given nucleic acid molecule. The same or different adapters can be linked to the respective ends of a nucleic acid molecule. In some implementations, the same adapter is linked to the respective ends of the nucleic acid molecule except that the nucleic acid tag differs. In some implementations, the adapter is a Y-shaped adapter in which one end is blunt ended or tailed as described herein, for joining to a nucleic acid molecule, which is also blunt ended or tailed with one or more complementary nucleotides. In still other example implementations, an adapter is a bell-shaped adapter that includes a blunt or tailed end for joining to a nucleic acid molecule to be analyzed. Other examples of adapters include T-tailed and C-tailed adapters.

Alignment. As used herein, “alignment” or “align” refers to determining whether at least two sequence representations have at least a threshold amount of homology. In one or more examples, the threshold amount of homology can be at least about 90%, at least about 91%, at least about 92%, at least about 93%, at least about 94%, at least about 95%, at least about 96%, at least about 97%, at least about 98%, at least about 99%, at least about 99.5%, or at least about 99.9%. In situations where two sequence representations have at least the threshold amount of homology, the two sequence representations can be referred to as being “aligned.”

Amplify. As used herein, “amplify” or “amplification” in the context of nucleic acids refers to the production of multiple copies of a polynucleotide, or a portion of the polynucleotide, starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” or “molecular barcode” in the context of nucleic acids refers to a nucleic acid molecule comprising a sequence that can serve as a molecular identifier. For example, individual “barcode” sequences can be added to each DNA fragment during next-generation sequencing (NGS) library preparation so that each read can be identified and sorted before the final data analysis.

Cancer Type: As used herein, “cancer type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Carrier Signal: As used herein, “carrier signal” refers to any intangible medium that is capable of storing, encoding, or carrying transitory or non-transitory instructions 502 for execution by the machine 500, and includes digital or analog communications signals or other intangible medium to facilitate communication of such instructions 502. Instructions 502 may be transmitted or received over the network 534 using a transitory or non-transitory transmission medium via a network interface device and using any one of a number of well-known transfer protocols.

Cell-Free Nucleic Acid: As used herein, “cell-free nucleic acid” refers to nucleic acids not contained within or otherwise bound to a cell or, in some implementations, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, IRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Nucleic Acids: As used herein, “cellular nucleic acids” means nucleic acids that are disposed within one or more cells at least at the point a sample is taken or collected from a subject, even if those nucleic acids are subsequently removed as part of a given analytical process.

Classification Region: As used herein, “classification region” refers to a genomic region that may show sequence-independent changes in neoplastic cells (e.g., tumor cells and cancer cells) or that may show sequence-independent changes in cfDNA from subjects having cancer relative to cfDNA from subjects in which cancer is not present. Examples of sequence-independent changes include, but are not limited to, changes in methylation rate (increases or decreases), nucleosome distribution, CTCF binding, transcription start sites, and regulatory protein binding regions. In one or more examples, sequence-independent changes in a classification region can indicate the presence of a single form of cancer in a subject. In one or more additional examples, sequence-independent changes in a classification region can correspond to the presence of multiple forms in a subject. The classification region can be enriched by one or more probes. In addition, the classification region can be defined by a pair of primer binding sites. Further, the classification region can be defined by a predetermined beginning genomic locus and a predetermined ending genomic locus. The classification region can include from about 25 nucleotides to about 250 nucleotides, from about 50 nucleotides to about 200 nucleotides, or from about 75 nucleotides to about 150 nucleotides. For instance, classification region can be a differentially methylated region. “Differentially methylated region” or “DMR” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation (e.g., a hypermethylated region/hypermethylated target region) in at least one cell or tissue type relative to the degree of methylation in the same region of DNA from at least one other cell or tissue type that contribute to cfDNA in healthy individuals, or from the same cell or tissue type from a healthy subject. In some embodiments, a differentially methylated region has a detectably lower degree of methylation (e.g., a hypomethylated region/hypomethylated target region) in at least one cell or tissue type relative to the degree of methylation in the same region of DNA from at least one other cell or tissue type, such as other immune cell types and/or cell types that contribute to cfDNA in healthy individuals, or from the same cell or tissue type from a healthy subject. In some embodiments, the classification regions comprise hypermethylated target regions and/or hypomethylated target regions.

Communications Network: As used herein, “communications network” refers to one or more portions of a network 114, 1034 that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network 114, 1034 or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

Confidence Interval: As used herein, “confidence interval” means a range of values so defined that there is a specified probability that the value of a given parameter lies within that range of values.

Control Sample: As used herein, “control sample” or “reference sample” refers to a sample obtained from individuals without known copy number variation.

Coverage: As used herein, “coverage” or “coverage metrics” refer to the number of nucleic acid molecules or sequencing reads that correspond to a particular genomic region of a reference sequence.

Deoxyribonucleic Acid or Ribonucleic Acid: As used herein, “deoxyribonucleic acid” or “DNA” refers to a natural or modified nucleotide which has a hydrogen group at the 2′-position of the sugar moiety. DNA can include a chain of nucleotides comprising four types of nucleotide bases: adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA can include a chain of nucleotides comprising four types of nucleotides: A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data”, “nucleic acid sequencing information”, “sequence information”, “sequence representation”, “nucleic acid sequence”, “nucleotide sequence”, “genomic sequence”, “genetic sequence”, “fragment sequence”, “sequencing read”, or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Differentially Methylated Region: As used herein, differentially methylated region” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation (e.g., a hypermethylated region) in at least one cell or tissue type, such as at least one immune cell type, relative to the degree of methylation in the same region of DNA from at least one other cell or tissue type, such as other immune cell types and/or cell types that contribute to cfDNA in healthy individuals, or from the same cell or tissue type from a healthy subject. In some embodiments, a differentially methylated region has a detectably lower degree of methylation (e.g., a hypomethylated region) in at least one cell or tissue type, such as at least one immune cell type, relative to the degree of methylation in the same region of DNA from at least one other cell or tissue type, such as other immune cell types and/or cell types that contribute to cfDNA in healthy individuals, or from the same cell or tissue type from a healthy subject.

Driver Mutation: As used herein, “driver mutation” means a mutation that drives cancer progression.

Epigenetic Target Regions: As used herein, “epigenetic target regions” refers to target regions that may show sequence-independent differences in different cell or tissue types (e.g., different types of immune cells) or in neoplastic cells (e.g., tumor cells and cancer cells) relative to normal cells; or that may show sequence-independent differences (i.e., in which there is no change to the nucleotide sequence, e.g., differences in methylation, nucleosome distribution, or other epigenetic features) in DNA, such as cfDNA, from different cell types or from subjects having cancer relative to DNA, such as cfDNA, from healthy subjects, or in cfDNA originating from different cell or tissue types that ordinarily do not substantially contribute to cfDNA (e.g., immune, lung, colon, etc.) relative to background cfDNA (e.g., cfDNA that originated from hematopoietic cells). Examples of sequence-independent changes include, but are not limited to, changes in methylation (increases or decreases), nucleosome distribution, cfDNA fragmentation patterns, CCCTC-binding factor (“CTCF”) binding, transcription start sites (e.g., with respect to any one of more of binding of RNA polymerase components, binding of regulatory proteins, fragmentation characteristics, and nucleosomal distribution), and regulatory protein binding regions. Epigenetic target region sets thus include, but are not limited to, hypermethylation target region sets, hypomethylation target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. An epigenetic target region set is a set of epigenetic target regions.

Hypermethylation: As used herein, “hypermethylation” refers to an increased level or degree of methylation of nucleic acid molecule(s) relative to the other nucleic acid molecules within a population (e.g., sample) of nucleic acid molecules from the same genomic locus. In some embodiments, hypermethylated DNA can include DNA molecules comprising at least 1 methylated cytosine, at least 2 methylated cytosines, at least 3 methylated cytosines, at least 5 methylated cytosines, or at least 10 methylated cytosines.

Hypomethylation: As used herein, “hypomethylation” refers to a decreased level or degree of methylation of nucleic acid molecule(s) relative to the other nucleic acid molecules within a population (e.g., sample) of nucleic acid molecules from the same genomic locus. In some embodiments, hypomethylated DNA includes unmethylated DNA molecules. In some embodiments, hypomethylated DNA can include DNA molecules comprising 0 methylated cytosine, at most 1 methylated cytosine, at most 2 methylated cytosines, at most 3 methylated cytosines, at most 4 methylated cytosines, or at most 5 methylated cytosines.

Immunotherapy: As used herein, “immunotherapy” refers to treatment with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Example agents include antibodies against any of PD-1, PD-2, PD-L1, PD-L2, CTLA-40, OX40, B7.1, B7He, LAG3, CD137, KIR, CCR5, CD27, or CD40. Other example agents include proinflammatory cytokines, such as IL-1β, IL-6, and TNF-α. Other example agents are T-cells activated against a tumor, such as T-cells activated by expressing a chimeric antigen targeting a tumor antigen recognized by the T-cell.

Indel: As used herein, “indel” refers to a mutation that involves the insertion or deletion of nucleotides in the genome of a subject.

Limit of Detection (LoD): As used herein, “limit of detection” means the smallest amount of a substance (e.g., a nucleic acid) in a sample that can be measured by a given assay or analytical approach.

Machine-Readable Medium: As used herein, “machine-readable medium” refers to a component, device, or other tangible media able to store instructions 502 and data temporarily or permanently and may include, but is not limited to, random-access memory (RAM), read-only memory (ROM), buffer memory, flash memory, optical media, magnetic media, cache memory, other types of storage (e.g., erasable programmable read-only memory (EEPROM)) and/or any suitable combination thereof. The term “machine-readable medium” may be taken to include a single medium or multiple media (e.g., a centralized or distributed database, or associated caches and servers) able to store instructions 502. The term “machine-readable medium” shall also be taken to include any medium, or combination of multiple media, that is capable of storing instructions 502 (e.g., code) for execution by a machine 500, such that the instructions 502, when executed by one or more processors 504 of the machine 500, cause the machine 500 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

Maximum MAF: As used herein, “maximum MAF” or “max MAF” refers to the maximum MAF (mutant allele fraction) of all somatic variants in a sample.

Methylation: As used herein, “methylation” or “DNA methylation” refers to addition of a methyl group to a nucleotide base in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence). In some embodiments, DNA methylation refers to addition of a methyl group to adenine, such as in N⁶-methyladenine. In some embodiments, DNA methylation is 5-methylation (modification of the 5th carbon of the 6-carbon ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

Methylation-Dependent Nuclease: As used herein, “methylation-dependent nuclease” refers to a nuclease that preferentially cuts methylated DNA relative to unmethylated DNA. For example, a methylation-dependent nuclease may cut at or near a recognition sequence such as a restriction site in a manner dependent on methylation of at least one of the nucleobases in the recognition sequence, such as a cytosine. In some embodiments, the nucleolytic activity of the methylation-dependent nuclease is at least 10, 20, 50, or 100-fold higher on a methylated recognition site relative to an unmethylated control in a standard nucleolysis assay. Methylation-dependent nucleases include methylation-dependent restriction enzymes.

Methylation-Dependent Restriction Enzyme: As used herein, “methylation-dependent restriction enzyme” or “MDRE” refers to a restriction enzyme that is dependent on methylation of the DNA (e.g. cytosine methylation) i.e., the presence or absence of methyl group in a nucleotide base alters the rate at which the enzyme cleaves the target DNA. In some embodiments, the methylation dependent restriction enzymes do not cleave the DNA if a particular nucleotide base is unmethylated at the recognition sequence. For example, MspJI is a methylation dependent restriction enzyme with a recognition sequence “mCNNR(N9)” and it does not cleave DNA if the absence of the methylated cytosine (mC) in the recognition sequence.

Methylation-Sensitive Nuclease: As used herein, “methylation-sensitive nuclease” refers to a nuclease that preferentially cuts unmethylated DNA relative to methylated DNA. For example, a methylation-sensitive nuclease may cut at or near a recognition sequence such as a restriction site in a manner dependent on lack of methylation of at least one of the nucleobases in the recognition sequence, such as a cytosine. In some embodiments, the nucleolytic activity of the methylation-sensitive nuclease is at least 10, 20, 50, or 100-fold higher on an unmethylated recognition site relative to a methylated control in a standard nucleolysis assay. Methylation-sensitive nucleases include methylation-sensitive restriction enzymes.

Methylation Sensitive Restriction Enzyme: As used herein, “methylation sensitive restriction enzyme” or “MSRE” refers to a restriction enzyme that is sensitive to the methylation status of the DNA (e.g. cytosine methylation) i.e., the presence or absence of methyl group in a nucleotide base alters the rate at which the enzyme cleaves the target DNA. In some embodiments, the methylation sensitive restriction enzymes do not cleave the DNA if a particular nucleotide base is methylated at the recognition sequence. For example, HpaII is a methylation sensitive restriction enzyme with a recognition sequence “CCGG” and it does not cleave DNA if the second cytosine in the recognition sequence is methylated.

Methylation rate: As used herein, “methylation rate” refers to the probability, likelihood, or percentage that a given base (for example: cytosine residue in a CpG) is methylated on a DNA molecule at a particular genomic region analyzed in the sample. In some embodiments, the methylation rate may be applied to a defined region that comprises one or more potentially methylated bases. In some embodiments, the methylation rate refers to the percentage of CpG residues methylated in a DNA molecule. In some embodiments, the methylation rate refers to the percentage of CpG residues methylated in molecules aligned to particular genomic position or genomic region. Methylation rate can be measured by a variety of methods including, but not limited to, either using bisulfite sequencing (any single base resolution like TAPS, EM-SEQ, etc.) or using partitioning (DNA molecule resolution). Methylation rate can be measured in different ways. One estimation can be by counting how many DNA fragments end up in each methylation dependent partition or by counting the number of converted CpGs per fragment in the case of bisulfite sequencing or any other base-level resolution sequencing methods. In addition, in the case of methylation dependent partitioning, the rate calculation can be normalized using a set of predefined regions with known methylation state (i.e., positive control regions and/or negative control regions) or spiked-in synthetic DNA with known methylation state, deriving rate-parametrized partition distributions and estimating the rate using a maximum likelihood approach. In one or more examples, the methylation rate can be determined by determining an abundance of sequencing reads that correspond to a portion of a genomic region. The portion of the genomic region can include a number of genomic locations of the genomic region for which at least a threshold number of sequencing reads overlap.

Methylation Status: As used herein, “methylation status” or “methylation state” can refer to the presence or absence of methyl group on a DNA base (e.g. cytosine) at a particular genomic position in a nucleic acid molecule. It can also refer to the degree of methylation in a nucleic acid sequence (e.g., highly methylated, low methylated, intermediately methylated or unmethylated nucleic acid molecules). The methylation status can also refer to the number of nucleotides methylated in a particular nucleic acid molecule.

Modified Nucleotide Specific Binding Reagent: As used herein, refers to a binding reagent that is specific for, or targets, modified nucleotides. For example, a modified nucleotide can be a nucleotide that has been methylated, thus, the binding reagent can be specific for a methylated nucleotide. Examples of binding reagents include, but are not limited to, a methyl binding domain (MBD) of a methylation binding protein (“MBP”) or variants thereof, an antibody (and antibody variants e.g., single chain antibodies), aptamers, or combinations thereof. Thus, as disclosed throughout, the use of MBD can be exchanged for any other modified nucleotide specific binding reagent, provided the modified nucleotide specific binding reagent has the desired specificity and affinity for the specific modified base of interest in the selected implementation.

Mutant Allele Fraction: As used herein, “mutant allele fraction”, “mutation dose,” or “MAF” refers to the fraction of nucleic acid molecules harboring an allelic alteration or mutation at a given genomic position in a given sample. MAF is generally expressed as a fraction or a percentage. For example, an MAF can be less than about 0.5, 0.1, 0.05, or 0.01 (i.e., less than about 50%, 10%, 5%, or 1%) of all somatic variants or alleles present at a given locus.

Mutation: As used herein, “mutation” refers to a variation from a known reference sequence and includes mutations such as, for example, single nucleotide variants (SNVs), copy number variants or variations (CNVs)/aberrations, insertions or deletions (indels), gene fusions, transversions, translocations, frame shifts, duplications, repeat expansions, and epigenetic variants. A mutation can be a germline or somatic mutation. In some examples, a reference sequence for purposes of comparison is a wildtype genomic sequence of the species of the subject providing a test sample, typically the human genome.

Mutation Caller. As used herein, “mutation caller” means an algorithm (embodied in software or otherwise computer implemented) that is used to identify mutations in test sample data (e.g., sequence information obtained from a subject).

Mutation Count: As used herein, “mutation count” or “mutational count” refers to the number of somatic mutations in a whole genome or exome or targeted regions of a nucleic acid sample.

Negative Control Region: As used herein, “negative control region”, refers to a genomic region that is expected to be unmethylated or hypomethylated in essentially all samples, regardless of whether the DNA is derived from a cancer cell or a normal cell.

Neoplasm: As used herein, the terms “neoplasm” and “tumor” are used interchangeably. They refer to abnormal growth of cells in a subject. A neoplasm or tumor can be benign, potentially malignant, or malignant. A malignant tumor is referred to as a cancer or a cancerous tumor.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequencing reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g. molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference sequence, a sub-sequence of one or both ends of a sequence, and/or length of a sequence) in combination with at least one molecular barcode. A sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

Partitioning: As used herein, “partitioning” refers to physically separating or fractionating a mixture of nucleic acid molecules in a sample based on a characteristic of the nucleic acid molecules. The partitioning can be physical partitioning of molecules. Partitioning can involve separating the nucleic acid molecules into groups or sets based on the level of epigenetic feature (for e.g., methylation). For example, the nucleic acid molecules can be partitioned based on the level of methylation of the nucleic acid molecules. In some embodiments, the methods and systems used for partitioning may be found in PCT Patent Application No. PCT/US2017/068329, which is hereby incorporated by reference in its entirety.

Partitioned set: As used herein, “partitioned set” or “partition” refers to a set of nucleic acid molecules partitioned into a set or group based on the differential binding affinity of the nucleic acid molecules or proteins associated with the nucleic acid molecules to a binding agent. A partitioned set may also be referred to as a subsample. The binding agent binds preferentially to the nucleic acid molecules comprising nucleotides with epigenetic modification. For example, if the epigenetic modification is methylation, the binding agent can be a methyl binding domain (MBD) protein. In some embodiments, a partitioned set can comprise nucleic acid molecules belonging to a particular level or degree of epigenetic feature (for e.g., methylation). For example, the nucleic acid molecules can be partitioned into three sets-one set for highly methylated nucleic acid molecules (first subsample, hyper partition, hyper partitioned set or hypermethylated partitioned set), a second set for low methylated nucleic acid molecules (second subsample, hypo partition, hypo partitioned set or hypomethylated partitioned set), and a third set for intermediate methylated nucleic acid molecules (third subsample, intermediate partitioned set, intermediately methylated partitioned set, residual partition, or residual partitioned set). In another example, the nucleic acid molecules can be partitioned based on the number of methylated nucleotides-one partitioned set can have nucleic acid molecules with nine methylated nucleotides, and another partitioned set can have unmethylated nucleic acid molecules (zero methylated nucleotides).

Polynucleotide: As used herein, “polynucleotide”, “nucleic acid”, “nucleic acid molecule”, “polynucleotide molecule”, or “oligonucleotide” refers to a linear polymer of nucleosides (including deoxyribonucleosides, ribonucleosides, or analogs thereof) joined by internucleosidic linkages. A polynucleotide can comprise at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Positive Control Region: As used herein, As used herein, “positive control region”, refers to a genomic region that is expected to be methylated or hypermethylated in essentially all samples, regardless of whether the DNA is derived from a cancer cell or a normal cell.

Probe: As used herein, “probe” refers to a polynucleotide comprising a functionality. The functionality can be a detectable label (fluorescent), a binding moiety (biotin), or a solid support (a magnetically attractable particle or a chip). Probes can include single-stranded DNA/RNA polynucleotides or double stranded DNA polynucleotides that hybridize to target nucleic acid sequences (e.g., SureSelect® probes, Agilent Technologies). Sequence capture using probes generally depends, in part, on the number of consecutive nucleotides in at least a portion of the target nucleic acid sequence that is complementary (or nearly complementary) to the sequence of the probe. In some examples, probes can correspond to driver mutations.

Processing: As used herein, the terms “processing”, “calculating”, and “comparing” can be used interchangeably. In certain applications, the terms refer to determining a difference, e.g., a difference in number or sequence. For example, gene expression, copy number variation (CNV), indel, and/or single nucleotide variant (SNV) values or sequences can be processed.

Processor. As used herein, “processor” refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC or any combination thereof. A processor may further be a multi-core processor having two or more independent processors (sometimes referred to as “cores”) that may execute instructions contemporaneously.

Promoter Region As used herein, “promoter region” refers to a DNA sequence recognized by the synthetic machinery of the cell, or introduced synthetic machinery, required to initiate the specific transcription of a gene.

Quantitative Measures: As used herein, “quantitative measures” refers to an absolute or relative measure. A quantitative measure can be, without limitation, a number, a statistical measurement (e.g., frequency, mean, median, standard deviation, or quantile), or a degree or a relative quantity (e.g., high, medium, and low). A quantitative measure can be a ratio of two quantitative measures. A quantitative measure can be a linear combination of quantitative measures. A quantitative measure may be a normalized measure.

Reference Sequence: As used herein, “reference sequence” refers to a known sequence used for purposes of comparison with experimentally determined sequences. For example, a known sequence can be an entire genome, a chromosome, or any segment thereof. A reference sequence can include at least about 20, at least about 50, at least about 100, at least about 200, at least about 250, at least about 300, at least about 350, at least about 400, at least about 450, at least about 500, at least about 1000, or more nucleotides. A reference sequence can align with a single contiguous sequence of a genome or chromosome or can include non-contiguous segments that align with different regions of a genome or chromosome. Example reference sequences, include, for example, human genome reference sequences, such as, hG19 and hG38.

Sample: As used herein, “sample” means anything capable of being analyzed by the methods and/or systems disclosed herein.

Sensitivity. As used herein, “sensitivity” means the probability of detecting the presence of a single nucleotide variant, an insertion, and a deletion at a given MAF and coverage and the probability of detecting the presence of a copy number variant at a given tumor fraction and coverage.

Sequencing: As used herein, “sequencing” refers to any of a number of technologies used to determine the sequence (e.g., the identity and order of monomer units) of a biomolecule, e.g., a nucleic acid such as DNA or RNA. Example sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing, MS-PET sequencing, and a combination thereof. In some implementations, sequencing can be performer by a gene analyzer such as, for example, gene analyzers commercially available from Illumina, Inc., Pacific Biosciences, Inc., or Applied Biosystems/Thermo Fisher Scientific, among many others.

Single Nucleotide Variant: As used herein, “single nucleotide variant” or “SNV” means a mutation or variation in a single nucleotide that occurs at a specific position in the genome.

Somatic Mutation: As used herein, “somatic mutation” means a mutation in the genome that occurs after conception. Somatic mutations can occur in any cell of the body except germ cells and accordingly, are not passed on to progeny.

Specifically binds: As used herein, “specifically binds” in the context of an probe or other oligonucleotide and a target sequence means that under appropriate hybridization conditions, the oligonucleotide or probe hybridizes to its target sequence, or replicates thereof, to form a stable probe: target hybrid, while at the same time formation of stable probe: non-target hybrids is minimized. Thus, a probe hybridizes to a target sequence or replicate thereof to a sufficiently greater extent than to a non-target sequence, to enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

Subject. As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Target Region: As used herein, “target region” refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity).

Threshold: As used herein, “threshold” refers to a predetermined value used to characterize experimentally determined values of the same parameter for different samples depending on their relation to the threshold.

Tumor Fraction: As used herein, “tumor fraction” refers to the estimate of the fraction of nucleic acid molecules derived from a tumor in a given sample. For example, the tumor fraction of a sample can be a measure derived from the max MAF of the sample or pattern of sequencing coverage of the sample or length of the cfDNA fragments in the sample or any other selected feature of the sample. In some instances, the tumor fraction of a sample is equal to the max MAF of the sample.

Variant: As used herein, a “variant” can be referred to as an allele. A variant is usually presented at a frequency of 50% (0.5) or 100% (1), depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

### DETAILED DESCRIPTION

Cancer is usually caused by the accumulation of mutations within genes of an individual's cells, at least some of which result in improperly regulated cell division. Such mutations can include single nucleotide variations (SNVs), gene fusions, insertions, transversions, translocations, and inversions. These mutations can also include copy number variations that correspond to an increase or a decrease in the number of copies of a gene within a tumor genome relative to an individual's noncancerous cells. An extent of mutations present in cell-free nucleic acids and an amount of mutated cell-free nucleic acids of a sample can be used as biomarkers to determine tumor progression, predict patient outcome, and refine treatment choices. In various examples, the extent of mutations present in cell-free nucleic acids can be indicated by tumor cells copy number and tumor fraction for a given sample.

Additionally, cancer can be indicated by non-sequence modifications, such as methylation. Examples of methylation changes in cancer include local gains of DNA methylation in the CpG islands at the TSS of genes involved in normal growth control, DNA repair, cell cycle regulation, and/or cell differentiation. This increased amount of methylation can be associated with an aberrant loss of transcriptional capacity of involved genes and occurs at least as frequently as point mutations and deletions as a cause of altered gene expression.

Thus, DNA methylation profiling can be used to detect aberrant methylation in DNA of a sample. The DNA can correspond to certain genomic regions (“differentially methylated regions” or “DMRs”) that are normally hypermethylated or hypomethylated in a given sample type (e.g., cfDNA from the bloodstream) but which may show an abnormal degree of methylation that correlates to a neoplasm or cancer, e.g., because of unusually increased contributions of tissues to the type of sample (e.g., due to increased shedding of DNA in or around the neoplasm or cancer) and/or from extents of methylation of the genome that are altered during development or that are perturbed by disease, for example, cancer or any cancer-associated disease.

Some methods of measuring DNA methylation, can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determination of tumor fraction estimates can impact diagnosis and/or treatment decisions for individuals.

The methods and systems described herein are directed to accurately generating information indicating the amounts of methylation of nucleic acids using data that indicates an amount of binding of nucleic acids to methyl binding domain (MBD). In various examples, the application is directed to systems and processes to determine an estimate for tumor fraction of a sample. In one or more examples, amounts of methylation of nucleic acids can be determined based on a strength of binding by the nucleic acids to methyl binding domain (MBD). The nucleic acids can be partitioned according to the strength of binding to MBD. Additionally, a number of cytosine-guanine (CG) regions for the nucleic acids can be determined. Amounts of methylation of classification regions of the nucleic acids can be determined based on the partition information associated with the nucleic acids and the number of cytosine-guanine regions of the nucleic acids. The classification regions can have differing amounts of methylation in tumor cells and non-tumor cells. The estimate for tumor fraction of the sample can be determined according to the amounts of methylation of the classification regions.

In at least some implementations, the methods, systems, techniques, and architectures can implement models that are configured to have at least one of parameters or weights that can be modified to more accurately fit to the methylation data provided to the models. The methods, systems, techniques, and architectures are also directed to implementing a number of optimization procedures during the training of the models to generate models that more accurately predict metrics indicating the presence or absence of tumors than other systems, methods, techniques, and architectures. Further, the methods, techniques, and processes used to generate the information used to produce the methylation data reduce the amount of noise present in the methylation data that leads to more accurate predictions of metrics that indicate the presence or absence of tumors than other methods, techniques, and processes.

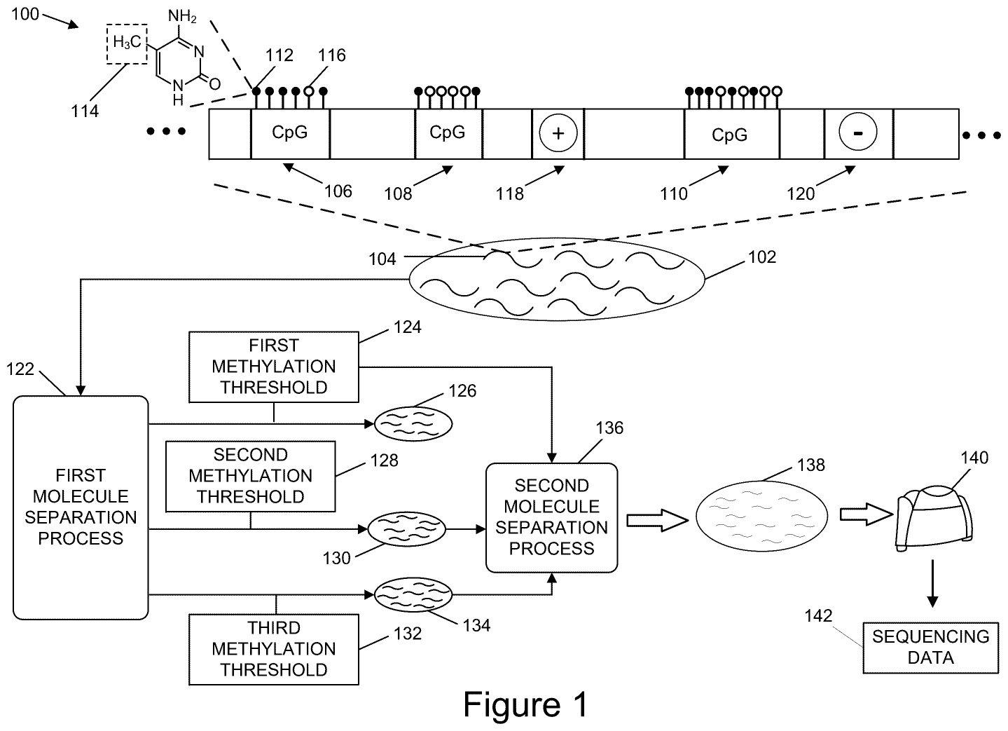

FIG. 1 is a diagrammatic representation of an example environment 100 that identifies nucleic acids that correspond to classification regions of a reference sequence, where the classification regions have at least a threshold number of CpGs, according to one or more implementations. In one or more examples, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

The environment 100 can include a sample 102. The sample 102 can be derived from a biological fluid obtained from a subject. For example, the sample 102 can be derived from blood obtained from a subject. In one or more additional examples, the sample 102 can be derived from tissue of a subject. In various examples, the sample 102 can be derived from multiple sources. To illustrate, the sample 102 can be derived from one or more fluids of a subject and/or from tissue of a subject. In one or more illustrative examples, the subject can be a mammal. In one or more additional illustrative examples, the subject can be a human. In one or more further illustrative examples, the subject can be a non-human mammal.

The sample 102 can include a number of nucleic acids 104. Individual nucleic acids 104 can include a number of regions that have at least a threshold number of cytosine molecules and guanine molecules. In one or more examples, individual nucleic acids 104 can include regions having at least a threshold number of cytosine-guanine dinucleotides. In various examples, at least a portion of the cytosine-guanine pairs included in the regions can be sequentially located in sequences of the nucleic acids 104. In one or more illustrative examples, a region of a nucleic acid having at least a threshold amount of cytosine-guanine pairs can be referred to herein as a “CG region” or a “CpG region.” In one or more examples, a CG region can include at least 200 CpG dinucleotides. In one or more illustrative examples, a CG region can include from 200 CpG dinucleotides to 5000 CpG dinucleotides, from 300 CpG dinucleotides to 3000 CpG dinucleotides, from 200 CpG dinucleotides to 2500 CpG dinucleotides, or from 500 CpG dinucleotides to 1500 CpG dinucleotides. Additionally, a CG region can have a GC percentage of at least 50% and an observed-to-expected CpG ratio of at least 60%. The observed-to-expected CpG ratio can be calculated where the observed CpG is the number of CpGs identified in a given genomic region and the expected CpGs is the number of cytosines multiplied by the number of guanines divided by the number of bases in the genomic region. The expected CpGs can also be calculated by:

((number of cytosines+number of guanines)/2)²/length of genomic region.

For example, a CG region can be determined using the techniques described by Gardiner-Garden M, Frommer M (1987). “CpG islands in vertebrate genomes”. Journal of Molecular Biology. 196 (2): 261-282. and/or Saxonov S, Berg P, Brutlag DL (2006). “A genome-wide analysis of CpG dinucleotides in the human genome distinguishes two distinct classes of promoters”. Proc Natl Acad Sci USA. 103 (5): 1412-1417.

In the illustrative example of FIG. 1, a portion of a sequence of an example nucleic acid 104 can include a first CG region 106, a second CG region 108, and a third CG region 110. Although the illustrative example of FIG. 1 illustrates a portion of a sequence of a nucleic acid 104 having three CG regions, nucleic acids 104 included in the sample 102 can have a different number of CG regions. For example, individual nucleic acids 104 included in the sample 102 can include at least 1 CG region, at least 5 CG regions, at least 10 CG regions, at least 25 CG regions, at least 50 CG regions, at least 100 CG regions, at least 250 CG regions, at least 500 CG regions, or at least 1000 CG regions.

Individual CG regions can correspond to a number of molecules with one or more methylated cytosines. In the illustrative example of FIG. 1, the CG region 106 can include a molecule with a methylated cytosine 112. In the illustrative example of FIG. 1, the molecule with a methylated cytosine 112 is 5-methylcytosine. Individual CG regions can also correspond to a number of molecules with an unmethylated cytosine. For example, the CG region 106 can include a molecule with an unmethylated cytosine 116. In various examples, at least a portion of the CG regions of a nucleic acid 104 can correspond to classification regions of a reference genome. Classification regions can correspond to genomic regions of a reference genome that correspond to non-sequence differences that are consistent with one or more biological conditions, such as one or more types of cancer. In at least some examples, the non-sequence differences can include one or more mutations that are consistent with one or more biological conditions. In one or more examples, a classification region can correspond to a genomic region of the reference sequence for which molecules derived from subjects having at least one form of cancer. In at least some examples, nucleic acid molecules having at least a threshold amount of methylated cytosines in at least one CG region (e.g., hypermethylated molecules) can be derived from subjects in which cancer is present and correspond to a classification. In one or more additional examples, nucleic acid molecules having less than a threshold amount of methylated cytosines (e.g., hypomethylated molecules) in at least one CG region can be derived from subjects in which cancer is present and correspond to a classification region.

In addition to the classification regions, the CG regions can include one or more positive control regions, such as positive control region 118. The positive control region 108 can be mapped to nucleic acid molecules having at least a threshold number of methylated cytosine molecules in at least one CG region and that are derived from subjects that are free of cancer and are derived from subjects in which cancer is present. In various examples, the positive control region 106 can be hypermethylated in cells derived from subjects that are free of cancer and also in cells derived from subjects in which cancer is present. The CG regions can also include one or more negative control regions, such as negative control region 120. The negative control region 120 can be mapped to nucleic acid molecules having less than a threshold number of methylated cytosine molecules in at least one CG region and that are derived from subjects that are free of cancer and also subjects in which cancer is present. In one or more illustrative examples, the negative control region 120 can be hypomethylated in subjects that are free of cancer and also in subjects in which cancer is present. In various examples, the positive control regions and the negative control regions can be used to perform normalization calculations. The normalization calculations can be performed to generate input data for one or more models that are implemented to determine tumor metrics for a given sample 102.

A first molecule separation process 122 can be performed. The first molecule separation process 122 can separate nucleic acids 104 included in the sample 102 based on an amount of methylated cytosines of the individual nucleic acids 104. In one or more examples, the first molecule separation process can separate nucleic acids 104 included in the sample 102 based on amounts of methylated cytosines included in CG regions of individual nucleic acids 104. In various examples, the first molecule separation process 122 can separate the nucleic acids 104 into a plurality of groups with individual groups corresponding to respective amounts of methylated cytosines of the nucleic acids 104.

In the illustrative example of FIG. 1, the first molecule separation process 122 can be performed in relation to a first methylation threshold 124. Performing the first molecule separation process 122 with regard to the first methylation threshold 124 can produce a first partition of nucleic acids 126. In one or more examples, the first methylation threshold 124 can indicate a first threshold number of molecules with a methylated cytosine located in CG regions of the nucleic acids 104. The first molecule separation process 122 can identify a number of nucleic acids 104 having fewer molecules with a methylated cytosine in CG regions than the first methylation threshold 124. In various examples, the first methylation threshold 124 can correspond to a first methylation rate.

The first molecule separation process 122 can also be performed with respect to a second methylation threshold 128. The second methylation threshold 128 can indicate an amount of methylated cytosines in one or more genomic regions of the nucleic acids 104 that is greater than the amount of methylated cytosines in the one or more regions corresponding to the first methylation threshold 124. The second methylation threshold 124 can indicate a number of molecules with a methylated cytosine per a number of nucleic acids. In one or more additional examples, the second methylation threshold 124 can correspond to a rate of methylation of nucleic acids that is greater than the rate of methylation that corresponds to the first methylation threshold 124. Performing the first molecule separation process 122 with respect to the second methylation threshold 128 can produce a second partition of nucleic acids 130. In one or more examples, the first molecule separation process 122 can identify nucleic acids 104 having a greater amount of methylated cytosines than the first methylation threshold 124 and having a lower amount of methylated cytosines than the second methylation threshold 128 to produce the second partition of nucleic acids 130.

Additionally, the first molecule separation process 122 can also be performed with respect to a third methylation threshold 132. The third methylation threshold 132 can indicate an amount of methylated cytosines in one or more genomic regions of the nucleic acids 104 that is greater than the amount of methylated cytosines in the one or more regions corresponding to the first methylation threshold 124 and greater than the amount of methylated cytosines in the one or more regions corresponding to the second methylation threshold 128. The third methylation threshold 132 can indicate a number of molecules with a methylated cytosine per a number of nucleic acids. In one or more additional examples, the third methylation threshold 132 can correspond to a rate of methylated cytosines that is greater than the rate of methylation that corresponds to the first methylation threshold 124 and greater than the rate of methylation that corresponds to the second methylation threshold 128. Performing the first molecule separation process 122 with respect to the third methylation threshold 132 can produce a third partition of nucleic acids 134. In one or more examples, the first molecule separation process 122 can identify nucleic acids 104 having a greater amount of methylated cytosines than nucleic acids 104 included in the second partition of nucleic acids 128. In this way, the amount of methylated cytosines of nucleic acids included in the first partition 122, the second partition 126, and the third partition 130 increases from the first partition 122 to the second partition 126 and increases from the second partition 126 to the third partition 130. In one or more illustrative examples, the first partition of nucleic acids 126 can be referred to as a hypomethylation partition, the second partition of nucleic acids 130 can be referred to as an intermediate partition, and the third partition of nucleic acids 134 can be referred to as a hypermethylation partition.

In one or more examples, the amount of methylated cytosines of nucleic acids can correspond to a strength of binding to methyl binding domain (MBD). In these scenarios, the first partition 126, the second partition 130, and the third partition 134 can be produced based on different strengths of binding to MBD for nucleotides having different amounts of methylated cytosines. In one or more examples, the first molecule separation process 122 can include a series of washes where the nucleic acids 104 are contacted with solutions having different concentrations of sodium chloride (NaCl).

Partitioning of the nucleic acids can be performed by contacting the nucleic acids with a modified nucleotide specific binding reagent, such as a MBD of a MBP. A modified nucleotide specific binding reagent can bind to 5-methylcytosine (5mC). The modified nucleotide specific binding reagent, such as a MBD, can be coupled to paramagnetic beads, such as Dynabeads® M-280 Streptavidin via a biotin linker. Partitioning into fractions with different extents of methylation can be performed by increasing the NaCl concentration in a series of washes. The sequences eluted from the modified nucleotide specific binding reagent are partitioned into two or more fractions (e.g., hypo, hyper) depending on which wash (e.g., NaCl concentration) eluted the sequences. Resulting partitions can include one or more of the following nucleic acid forms: double-stranded DNA (dsDNA), shorter DNA fragments and longer DNA fragments.

The binding of the nucleic acids with the modified nucleotide specific binding reagent can be a function of number of methylated (or modified) sites per molecule, with molecules having more methylation eluting under increased salt concentrations. To elute the DNA into distinct populations based on the extent of methylation, one can use a series of elution buffers of increasing NaCl concentration. Salt concentrations can, in one or more implementations, range from about 100 nM to about 2500 mM NaCl. In various implementations, the process results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population (hypo partition). For example, the first partition 126 can be representative of the hypomethylated form of DNA is that which remains unbound at a low salt concentration. In one or more illustrative examples, the concentration of NaCl of the solution used to produce the first partition 126 can be about 100 nM, about 120 nM, about 140 nM, about 160 nM, about 180 nM, about 200 nM. or about 250 nM. The second partition 130 can be referred to as a “residual partition” or an “intermediate partition” and can be representative of intermediate methylated DNA is eluted using an intermediate salt concentration, e.g., between 100 mM and 2000 mM concentration. In one or more additional illustrative examples, the concentration of NaCl of the solution used to produce the second partition 130 can be from about 100 mM to about 500 mM, from about 100 mM to about 1000 mM, from about 100 mM to about 1500 mM, from about 250 mM to about 1000 mM, from about 250 mM to about 1500 mM, from about 500 mM to about 1500 mM, from about 250 mM to about 2000 mM, from about 500 mM to about 2000 mM, or from about 1000 mM to about 2000 mM. This is also separated from the sample. The third partition 134 can be representative of hypermethylated form of DNA (hyper partition) and is eluted using a high salt concentration, e.g., at least about 2000 mM. In one or more further illustrative examples, the concentration of NaCl of the solution used to produce the third partition 134 can be from about 2000 mM to about 5000 mM, from about 2000 mM to about 4000 mM, from about 2000 mM to about 3500 mM, from about 2000 mM to about 3000 mM, or from about 2500 mM to about 4000 mM.

In various examples, the first partition 126 can correspond to a first range of binding strengths of nucleic acids to MBD and to a first range of methylated CG regions and the second partition 130 can correspond to a second range of binding strengths of nucleic acids to MBD and to a second range of methylated CG regions. The first range of binding strengths can be less than the second range of binding strengths. In one or more scenarios, a first solution having a first NaCl concentration can separate a first group of nucleic acids having the first range of binding strengths from MBD and a second solution having a second NaCl concentration can separate a second group of nucleic acids having the second range of binding strengths from MBD with the second NaCl concentration being greater than the first NaCl concentration. Additionally, the third partition 134 can correspond to a third range of binding strengths and a third range of methylated CG regions. The third range of binding strengths can be greater than the first range of binding strengths and the second range of binding strengths. In one or more instances, a third solution having a third NaCl concentration can separate a third group of nucleic acids having the third range of binding strengths from NaCl. The third NaCl concentration can be greater than the first NaCl concentration and the second NaCl concentration.

In one or more illustrative examples, a plurality of nucleic acids derived from at least one of blood or tissue of a subject can be combined with a solution including an amount of MBD to produce a nucleic acid-MBD solution. A first wash of the nucleic acid-MBD solution can be performed with a first solution including a first NaCl concentration to produce a first nucleic acid fraction and a first residual solution. The first nucleic acid fraction can include a first portion of the plurality of nucleic acids and the first residual solution can include a second portion of the plurality of nucleic acids. In one or more examples, the first portion of the plurality of nucleic acids can have a first range of binding strengths to MBD that are less than a second range of binding strengths to MBD of the second portion of the plurality of nucleic acids.

Additionally, a second wash of the first residual solution can be performed with a second solution including a second concentration of NaCl that is greater than the first concentration of NaCl to produce a second nucleic acid fraction and a second residual solution. The second nucleic acid fraction can include a first subset of the second portion of the plurality of nucleic acids and the second residual solution can include a second subset of the second portion of the plurality of nucleic acids. The first subset of the second portion of the plurality of nucleic acids can have a third range of binding strengths to MBD that are less than a fourth range of binding strengths to MBD of the second subset of the second portion of the plurality of nucleic acids. Further, a third wash of the second residual solution can be performed with a third solution including a third concentration of NaCl that is greater than the second concentration of NaCl to produce a third nucleic acid fraction that includes the second subset of the second portion of the plurality of nucleic acids.

Subsequent to the first wash, the second wash, and the third wash a determination can be made that the first portion of the plurality of nucleic acids are associated with the first partition 126. The first portion of the plurality of nucleic acids can be attached with molecular barcodes from a first set of molecular barcodes indicating the first partition 126. In this way, a sequencing read that corresponds to the first partition 126 can be identified based on determining that the sequencing read includes the first molecular barcode. In addition, a determination can be made that the first subset of the second portion of the plurality of nucleic acids is associated with an additional partition of the plurality of partitions. In these situations, a second set of molecular barcodes different from the first set of molecular barcodes can be attached to the second portion of the plurality of nucleic acids with the second molecular barcode indicating the additional partition. As a result, a sequencing read that corresponds to the additional partition can be identified based on determining that the sequencing read includes one or more molecular barcodes from among the second set of molecular barcodes. Further, a determination can be made that the second subset of the second portion of the plurality of nucleic acids is associated with the second partition 130. A third set of molecular barcodes different from the first set of molecular barcodes and the second set of molecular barcodes can then be attached to the second subset of the second portion of the plurality of nucleic acids where the third set of molecular barcodes indicate the second partition 130. In these instances, a sequencing read that corresponds to the second partition 130 can be identified based on determining that the sequencing read includes a third molecular barcode from among the third set of molecular barcodes.

In at least some examples, the first molecule separation process 122 can result in nucleic acids being present in at least one of the first partition 126, the second partition 130, or the third partition 134 having an amount of methylation that is different from the amount of methylation of the other nucleic acids in the respective partition. For example, the first partition 126 can include a number of nucleic acids having amounts of methylation that correspond to the amounts of methylation of nucleic acids included in at least one of the second partition 130 or the third partition 134. Additionally, at least one of the second partition 130 or the third partition 134 can include nucleic acids having amounts of methylation that correspond to the amounts of methylation of nucleic acids included in the first partition 126. The presence of nucleic acids in at least one of the first partition 126, the second partition 130, or the third partition 134 that do not correspond to the amounts of methylation of at least a majority of the other nucleic acids included in the respective partition can cause data noise when performing computational operations with respect to sequence reads produced from nucleic acids included in the first partition 126, the second partition 130, and the third partition 134. The data noise can result in inaccuracies with respect to calculations made based on sequence reads derived from nucleic acids included in the first partition 126, the second partition 130, and the third partition 134.

To reduce or eliminate data noise associated with nucleic acids being present in at least one of the first partition 126, the second partition 130, or the third partition 134 that have amounts of methylation that are not consistent with the amounts of methylation of at least a majority of other molecules included in the respective partitions, a second molecule separation process 136 can be performed after the first molecule separation process 122. The second molecule separation process 136 can be performed with respect to nucleic acids included in the first partition 126, nucleic acids included in the second partition 130, and nucleic acids included in the third partition 134. In one or more examples, the second molecule separation process 136 can include performing digestion of the nucleic acids included in the first partition 126 using methylation dependent restriction enzyme (MDRE) and nucleic acids included in the second partition 130 and the third partition 134 can be digested using methylation sensitive restriction enzyme (MSRE). Digestion of the nucleic acids included in the first partition 126 with MDRE can result in separation of nucleic acids included in the first partition having amounts of methylation corresponding to the second partition 130 and the third partition 134 from nucleic acids having amounts of methylation corresponding to the first partition. Additionally, digestion of nucleic acids included in the second partition 130, and the third partition 134 with MSRE can result in separation of the nucleic acids having amounts of methylation corresponding to the first partition 126 from the nucleic acids of the second partition 130 and the nucleic acids of the third partition 134. By removing nucleic acids from the first partition 126 having amounts of methylation that correspond to the second partition 130 and the third partition 134 and by removing nucleic acids from the second partition 130 and the third partition 134 that have amounts of methylation that correspond to the first partition 126, an additional group of nucleic acids 138 can be produced. The additional group of nucleic acids 138 can include nucleic acids corresponding to methylation amounts of the second partition 130 and the third partition 134 with a minimal amount or no nucleic acids having amounts of methylation corresponding to the first partition 126. For example, less than 50% of the nucleic acids included in the additional group 138 can have amounts of methylation that correspond to the second partition 130 and the third partition 134, at least 50% of the nucleic acids included in the additional group 138 can have amounts of methylation that correspond to the second partition 130 and the third partition 134, at least 60% of the nucleic acids included in the additional group 138 can have amounts of methylation that correspond to the second partition 130 and the third partition 134, at least 70% of the nucleic acids included in the additional group 138 can have amounts of methylation that correspond to the second partition 130 and the third partition 134, at least 90% of the nucleic acids included in the additional group 138 can have amounts of methylation that correspond to the second partition 130 and the third partition 134, at least 95% of the nucleic acids included in the additional group 138 can have amounts of methylation that correspond to the second partition 130 and the third partition 134, at least 97% of the nucleic acids included in the additional group 138 can have amounts of methylation that correspond to the second partition 130 and the third partition 134, at least 99% of the nucleic acids included in the additional group 138 can have amounts of methylation that correspond to the second partition 130 and the third partition 134, at least 99.5% of the nucleic acids included in the additional group 138 can have amounts of methylation that correspond to the second partition 130 and the third partition 134, or at least 99.9% of the nucleic acids included in the additional group 138 can have amounts of methylation that correspond to the second partition 130 and the third partition 134.

The architecture 100 can include a sequencing machine 140. In one or more examples, the sequencing machine 140 can be any of a number of sequencing machines that can perform one or more sequencing operations that amplify nucleic acids present in a sample 104. In various examples, the sequencing machine 140 can perform next-generation sequencing operations. In one or more examples, the sample 104 can include an amount of at least one bodily fluid extracted from a subject. In one or more additional examples, the sample 104 can include a tissue sample that is obtained from a subject.

In one or more examples, prior to sequencing, the extracted polynucleotides can be partitioned into two or more partitions based on the binding strength of the of binding strengths of polynucleotides to MBD. A blunt-end ligation can be performed on the partitioned polynucleotides and adapters, as well as tags (e.g., molecular barcodes) can be added to the partitioned polynucleotides. The tagged polynucleotides in the one or more partitions (e.g. hyper and/or intermediate partitions) can be treated with one or more methylation sensitive restriction enzymes (MSREs). In some examples, the hypo partition can be treated with one or more methylated dependent restriction enzymes (MDREs). Post the MSRE and/or MDRE treatment, the molecules can also be enriched by causing hybridization between the extracted polynucleotides and probes that correspond to target regions of a reference sequence. The enrichment process can identify thousands, hundreds of thousands, up to millions of polynucleotides that correspond to on-target regions associated with the probes.

Subsequent and/or prior to the enrichment process, the molecules can be amplified according to one or more amplification processes. The one or more amplification processes can produce thousands, up to millions of copies of individual nucleic acid molecules. In one or more examples, a portion of the unenriched polynucleotides can be amplified, in some instances, but not to the extent that the enriched polynucleotides are amplified. The one or more amplification processes can generate an amplification product that undergoes one or more sequencing operations. After performing one or more sequencing operations with respect to the sample 104, the sequencing machine 140 can produce a sequencing data 142.

The sequencing data 142 can include alphanumeric representations of the nucleic acids included in an amplification product. For example, the sequencing data 142 can include, for individual nucleic acids of the amplification product, data that corresponds to a string of letters that represent the respective chains of nucleotides that correspond to the individual nucleic acids.

The sequencing data 142 can be stored in one or more data files. For example, the sequencing data 142 can be stored in a FASTQ file that comprises a text-based sequencing data file format storing raw sequence data and quality scores. In one or more additional examples, the sequencing data 142 can be stored in a data file according to a binary base call (BCL) sequence file format. In one or more further examples, the sequencing data 142 can be stored in a BAM file. In one or more examples, the sequencing data 142 can comprise at least about one gigabyte (GB), at least about 2 GB, at least about 3 GB, at least about 4 GB, at least about 5 GB, at least about 8 GB, or at least about 10 GB. An individual sequence representation included in the sequencing data 106 can be referred to herein as a “read” or a “sequencing read.” In various examples, individual first nucleic acids included in the pool 138 can correspond to multiple sequence representations included in the sequencing data 142 as a result of the amplification of the individual first nucleic acids. In one or more additional examples, individual second nucleic acids included in the pool 138 can correspond to a single sequence representation included in the sequencing data 142 as a result of the absence of amplification of the individual second nucleic acids.

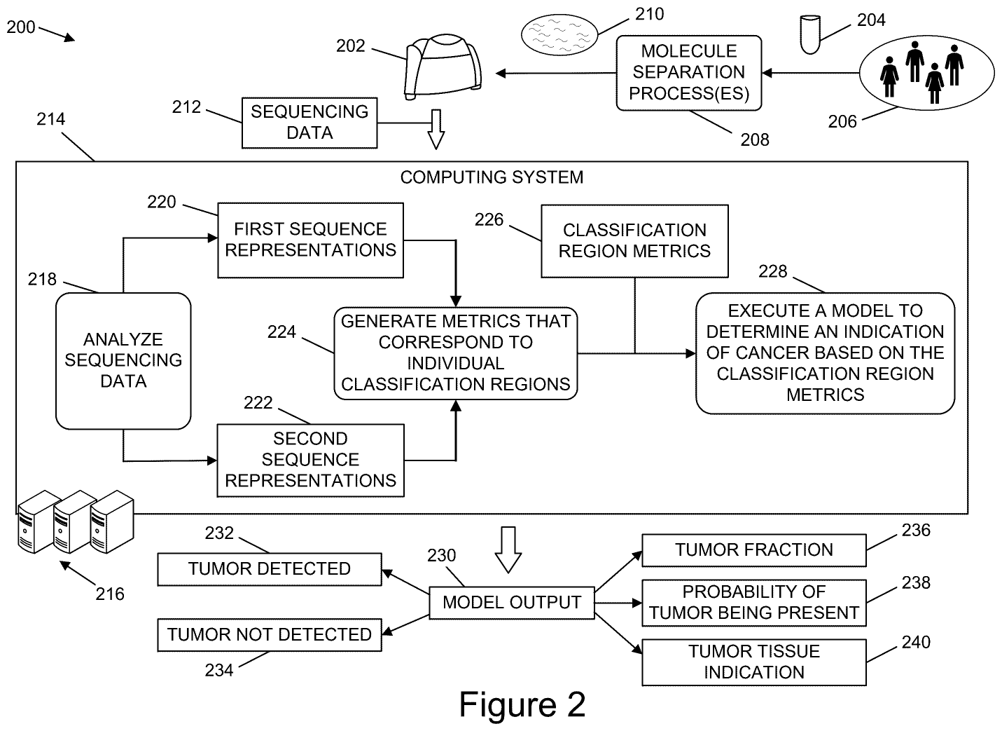

FIG. 2 is an example architecture 200 to analyze sequencing data to determine one or more metrics indicating the presence of a tumor in subjects, in accordance with one or more implementations. The architecture 200 can include one or more sequencing machines 202 that perform one or more sequencing operations with respect to a number of samples 204. The one or more samples 204 can be obtained from subjects 206. In one or more illustrative examples, a first portion of the subjects 206 can be free of cancer. That is, a tumor is not detected in the first portion of the subjects 206. Additionally, a tumor can be present in a second portion of the subjects 206.

One or more molecule separation processes 208 can be performed with respect to the samples 204. The one or more separation processes 208 can correspond to separating nucleic acid molecules into a number of partitions based on the characteristics of the nucleic acid molecules. Examples of characteristics that can be used for partitioning nucleic acid molecules include multiple different nucleotide modifications, methylation level, nucleosome binding, sequence mismatch, immunoprecipitation, and/or proteins that bind to DNA. In one or more illustrative examples, a heterogeneous population of nucleic acid molecules can be partitioned into nucleic acid molecules with one or more epigenetic modifications and without the one or more epigenetic modifications. Examples of epigenetic modifications include, but are not limited to, presence or absence of methylation; level of methylation, hydroxymethylation, and type of methylation (5′ cytosine or 6 methyladenine).

Prior to the one or more molecule separation processes 208, nucleic acid molecules can be extracted from a sample 204. In one or more implementations, the nucleic acid molecules comprise cell-free nucleic acids (e.g., cell-free DNA). In various implementations, the sample 204 can be a sample selected from one or more of blood, plasma, serum, urine, fecal, saliva samples, combinations thereof, and/or the like. In one or more additional examples, the sample 204 can comprise a sample selected from one or more of whole blood, a blood fraction, a tissue biopsy, pleural fluid, pericardial fluid, cerebrospinal fluid, and peritoneal fluid. In one or more illustrative examples, the cell-free nucleic acid molecules can be extracted from the sample 204 where the sample 204 is obtained from a subject 206 known to have cancer (e.g., a cancer patient), or a subject 206 suspected of having cancer.

The extraction of nucleic acid molecules from the sample 204 can include implementing one or more cell lysis techniques to cleave the membranes of cells included in the sample 204 and applying one or more proteases to break down proteins included in the sample 204. The extraction of nucleic acid molecules from the sample 204 can also include a number of washing and/or elution techniques to separate the nucleic acid molecules from other components included in the sample 204. In various examples, thousands, up to millions, up to billions of nucleic acid molecules can be extracted from the sample 204 prior to being subjected to the one or more separation processes 208.

The nucleic acid molecules extracted from samples 204 can include molecules having varying levels of methylation. Methylation can occur from any one or more post-replication or transcriptional modifications. Post-replication modifications include modifications of the nucleotide cytosine, including, but not limited to, 5-methylcytosine, 5-hydroxymethylcytosine, 5-formylcytosine and 5-carboxylcytosine. The one or more molecule separation processes 208 can separate nucleic acid molecules extracted from samples 204 into a number of partitions with individual partitions corresponding to different levels of methylation. For example, the molecule separation processes 208 can produce a first partition of nucleic acid molecules having first levels of methylation, a second partition of nucleic acid molecules having second levels of methylation, and a third partition of nucleic acid molecules having third levels of methylation. In various examples, the second levels of methylation can be greater than the first levels of methylation and the third levels of methylation can be greater than the first levels of methylation and the second levels of methylation. In one or more illustrative examples, the one or more molecule separation processes 208 can include the first molecule separation process 122 and the second molecule separation process 136 of FIG. 1.

The one or more molecule separation processes 208 can produce a pool 210 that includes a portion of the nucleic acid molecules extracted from one or more samples 204 and subjected to the one or more molecule separation processes 208. For example, the pool 210 can include a number of nucleic acid molecules having the second levels of methylation and a number of nucleic acid molecules having the third levels of methylation. Thus, the nucleic acid molecules included in the pool 210 can have at least a threshold amount of methylation. In one or more illustrative examples, the nucleic acid molecules included in the pool 210 can have at least a threshold amount of methylation in CG regions of the nucleic acid molecules.

The one or more sequencing machines 202 can perform one or more sequencing operations to produce sequencing data 212 that corresponds to the pool 210. The architecture 200 can include a computing system 214 that obtains the sequencing data 212 from the one or more sequencing machines 202 and analyzes the sequencing data 212. For example, the computing system 214 can analyze the sequencing data 212 to determine one or more metrics indicating that a tumor may be present in a subject 206 that provided at least one sample 204. The computing system 214 can include one or more computing devices 216. The one or more computing devices 216 can include at least one of one or more desktop computing devices, one or more mobile computing devices, or one or more server computing device. In various examples, at least a portion of the one or more computing devices 216 can be included in a remote computing environment, such as a cloud computing environment. In one or more examples, the computing system 214 and the sequencing machine 202 can be owned, operated, maintained, and/or controlled by a single organization. In one or more additional examples, the computing system 214 and the sequencing machine 202 can be owned, operated, maintained, and/or controlled by multiple organizations.

At operation 218, the computing system 214 can analyze the sequencing data 212. Analyzing the sequencing data 212 can include determining one or more first sequence representations 220 included in the sequencing data 212 that correspond to one or more classification regions of a reference sequence. The one or more classification regions can correspond to genomic regions of a reference sequence that are mapped to nucleic acid molecules having an amount of methylation in cfDNA obtained from subjects in which cancer is present relative to an amount of methylation of the molecules that map to the same genomic regions of the reference sequence in cfDNA obtained from subjects in which a tumor is not present. In at least some examples, the amount of methylation present in nucleic acid molecules that map to a classification region and are derived from subjects in which cancer is present is less than the amount of methylation present in nucleic acid molecules that map to the classification region and are derived from subjects in which cancer is not present. In one or more additional examples, the amount of methylation present in nucleic acid molecules that map to a classification region and are derived from subjects in which cancer is present is greater than the amount of methylation present in nucleic acid molecules that map to the classification region and are derived from subjects in which cancer is not present. The one or more classification regions can also include at least a threshold amount of cytosine-guanine content. In various examples, the one or more classification regions can include a series of cytosine-guanine (CG) pairs in 5′→3′ direction (CpG sites), such as at least 3 CpG sites, at least 5 CpG sites, at least 8 CpG sites, at least 10 CpG sites, at least 12 CpG sites, at least 15 CpG sites, at least 18 CpG sites, or at least 20 CpG sites.

In addition, the computing system 214 can analyze the sequencing data 212 to determine one or more second sequence representations 222 that correspond to one or more control regions of a reference sequence. The one or more control regions can include one or more positive control regions and/or one or more negative control regions. In various examples, a positive control region can comprise a genomic region of a reference sequence having at least a threshold amount of molecules with a methylated cytosine and including at least a threshold number of CpG sites. A positive control region can correspond to nucleic acid molecules having at least a threshold amount of methylation in one or more CG regions and that are obtained from subjects in which cancer is present and in samples obtained from subjects in which a tumor is not present. In at least some examples, the threshold amount of methylation can correspond to at least 5, at least 6, at least 7, at least 8, at least 9, at least 10, at least 11, at least 12, at least 15 or more CpGs being methylated in nucleic acid molecules. In one or more illustrative examples, positive control regions can be mapped to nucleic acid molecules that are hypermethylated in one or more CG regions and are derived from samples obtained from both subjects in which cancer is present and subjects in which cancer is not present. In one or more examples, a negative control region can comprise a genomic region of a reference sequence having less than a threshold amount of molecules with a methylated cytosine and at least a threshold number of CpG sites. A negative control region can correspond to nucleic acid molecules having less than an additional threshold amount of methylation in one or more CG regions and that are obtained from subject in which cancer is present and in samples obtained from subjects in which a tumor is not present. In various examples, the additional threshold amount of methylation can correspond to no greater than 1, no greater than 2, no greater than 3, no greater than 4, no greater than 5, no greater than 6, or no greater than 7 CpGs being methylated in nucleic acid molecules. In one or more additional illustrative examples, negative control regions can be mapped to nucleic acid molecules that are hypomethylated in one or more CG regions and are derived from samples obtained from both subjects in which cancer is present and subjects in which cancer is not present.

In one or more illustrative examples, the first sequence representations 220 can be determined by aligning sequence representations included in the sequencing data 212 with one or more classification regions of a reference sequence. In addition, the second sequence representations 222 can be determined by aligning sequence representations included in the sequencing data 212 with one or more control regions of a reference sequence. The alignment process can identify the first sequence representations 220 by determining a number of sequence representations included in the sequencing data 212 that correspond to one or more classification regions of the reference sequence. Further, the alignment process can identify the second sequence representations 222 by determining a number of sequence representations that correspond to one or more control regions of the reference sequence.

In one or more illustrative examples, the alignment process can determine an amount of homology between individual sequence representations included in the sequence data 212 and portions of the reference sequence. The amount of homology between a given sequence representation and the reference sequence can indicate a number of positions of the reference sequence that have the same nucleotide as corresponding positions of the given sequence representation. The computing system 214 can determine that a sequence representation is aligned with a portion of a reference sequence based on determining that the sequence representation and the portion of the reference sequence have at least a threshold amount of homology. In scenarios where a sequence representation has at least the threshold amount of homology with respect to multiple portions of the reference sequence, the portion of the reference sequence having the greatest amount of homology with the sequence representation can be determined to be aligned with the sequence representation.

The amount of homology between a given sequence representation and a portion of a reference sequence can be determined using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Genetics Computer Group, University Research Park, Madison Wis.), using default settings, which uses the algorithm of Needleman and Wunsch (J. Mol. Biol. 48; 443-453 (1970)). The amount of homology between a sequence representation and a portion of the reference sequence can also be determined using a Burrows-Wheeler aligner (Li, H., & Durbin, R. (2009). Fast and accurate short read alignment with Burrows-Wheeler transform. Bioinformatics, 25 (14), 1754-1760).

In one or more examples, after the sequence representations included in the sequencing data 212 have been aligned with a reference sequence, the aligned sequence representations can be analyzed to identify one or more groups of sequence representations. For example, individual aligned sequence representations can correspond to individual sequencing reads that are included in the sequencing data 212. In these scenarios, the aligned sequence representations can include multiple reads that correspond to a single nucleic acid molecule included in the sample pool 210. In one or more additional examples, the aligned sequence representations can correspond to individual nucleic acid molecules included in the pool 210. In these situations, the computing system can determine a group of reads included in the sequence data 212 that correspond to an individual nucleic acid molecules included in the pool 210 based on molecular barcodes that are common to each group of sequencing reads. That is, individual nucleic acid molecules included in the pool 210 can be encoded with molecular barcodes that uniquely identify the individual nucleic acid molecules and, in at least some cases, the individual nucleic acid molecules can be represented by multiple sequencing reads included in the sequencing data 212. Accordingly, when multiple sequence representations are present in the sequencing data 212 that correspond to a single nucleic acid molecule included in the pool 210, the computing system 214 can group the multiple sequence representations together. In various examples, the groups of sequence representations that correspond to a single nucleic acid molecule included in the pool 210 can be referred to herein as “families.” Additionally, start and stop positions with respect to the reference sequence of the aligned sequence representations having a common molecular barcode can be used to group the sequence representations that correspond to individual nucleic acids included in the pool 210. In one or more illustrative examples, an individual sequence representation that represents a family of sequence representations that corresponds to a single nucleic acid molecule included in the pool 210 can be referred to herein as a “consensus sequence representation.”

At operation 224, the computing system 214 can analyze the first sequence representations and the second sequence representations 222 to generate metrics that correspond to individual classification regions. In the illustrative example of FIG. 2, the computing system 214 can analyze the first sequence representations 220 and the second sequence representations 222 to generate classification region metrics 226. The classification region metrics 226 can include quantitative measures determined based on a number of first sequence representations 220 having at least a threshold amount of methylated cytosines. In one or more illustrative examples, the classification region metrics 226 can include quantitative measures determined based on a number of sequencing reads corresponding to a number of the first sequence representations 220 having at least a threshold amount of methylated cytosines located. In one or more additional illustrative examples, the classification region metrics 226 can include quantitative measures determined based on a number of nucleic acid molecules that correspond to a number of the first sequence representations 220. In various examples, the classification region metrics 226 can include quantitative measures determined based on a number of first sequence representations 220 having at least a threshold amount of methylated cytosines and a number of second sequence representations 222 that correspond to control regions of a reference sequence. In one or more further illustrative examples, the classification region metrics 226 can include quantitative measures related to a ratio of a number of first sequence representations 220 having at least a threshold amount of methylated cytosines in relation to a number of second sequence representations. In at least some examples, the sequence representations of the second sequence representations 222 used by the computing system 214 to generate quantitative measures included in the classification region metrics 226 can include sequence representations that correspond to positive control regions of a reference sequence.

The classification region metrics 226 can also be determined by performing one or more normalization operations with respect to quantitative measures generated by the computing system 214 using at least one of the first sequence representations 220 and the second sequence representations 222. For example, a logarithm calculation can be performed with respect to quantitative measures generated by the computing system 214 using at least one of the first sequence representations 220 or the second sequence representations 222. Additionally, the classification region metrics 226 can be determined by adding a pseudocount to quantitative measures determined by the computing system 214 using at least one of the first sequence representations 220 or the second sequence representations 222. In one or more illustrative examples, the one or more normalization operations can include determining quantitative measures that correspond to a ratio of first sequence representations 220 for an individual classification region with respect to a number of second sequence representations 222 that correspond to positive control regions of a reference sequence.

In one or more illustrative examples, the computing system 214 can determine a number of the first sequence representations 220 that correspond to individual classification regions of a reference sequence and that have at least a threshold amount of methylated cytosines located in the individual classification regions. In these scenarios, the computing system 214 can determine individual classification region metrics 226 for individual classification regions. In addition, the computing system 214 can determine a number of the second sequence representations 222 that correspond to positive control regions. In at least some examples, the computing system 214 can, for individual classification regions, determine a ratio including a number of first sequence representations 220 that correspond to the individual classification region and that have at least a threshold amount of molecules with a methylated cytosine in the classification region in relation to a total number of the second sequence representations 222 the correspond to positive control regions of a reference sequence. In one or more examples, the computing system 214 can add a value of a pseudocount to the ratio to determine a classification region metric 226 for the individual classification region. The value of the pseudocount can be at least 1, at least 1.2, at least 1.4, at least 1.6, at least 1.8, or at least 2. Further, the computing system 214 can perform a log base 10 operation with respect to the combination of the ratio and the pseudocount to determine a classification region metric 226 for an individual classification region. In at least some illustrative examples, the computing system 214 can determine at least a portion of the classification region metrics according to the following equation:

$$Score of region i = {log}_{10} ( \frac{{x}_{i}}{{x}_{positive \_ control}} + pseudocount ) , \qquad ( 1 )$$

where xᵢ is a total number of first sequence representations 220 for an individual classification region, i, having at least a threshold amount of methylated cytosines included in the region, I, and x_{positive_control} is a total number of the second sequence representations 222 that correspond to positive control regions of a reference sequence.

At operation 228, the computing system 214 can execute a model to determine an indication of cancer based on the classification region metrics 226. In the illustrative example of FIG. 2, the computing system 214 can execute a model using the classification region metrics 226 to generate model output 230. In one or more examples, the model output 230 can indicate a status of tumor detection 232 or a status of tumor not detected 234 in relation to a sample 204 provided by a subject 206. In one or more additional examples, the computing system 214 can execute a model to determine an estimate of tumor fraction 236 for a sample 204. In one or more further examples, the computing system 214 can execute a model to determine a probability of a tumor being present in a subject 206 that provided a sample 204.

In one or more examples, the model can include a classification model that implements one or more machine learning techniques. In one or more illustrative examples, the model can include a linear regression model. In various examples, the model can be executed to determine a probability of a tumor being present 238 in a subject 206 that provided a sample 204 based on the classification region metrics 226. In one or more illustrative examples, the computing system 214 can execute the model to determine weights for individual classification regions. The weights for individual classification regions can be different. For example, the computing system 214 can determine that a first weight of a first classification region metric 226 for a first classification region is different from a second weight of a second classification region metric 226 for a second classification region. In at least some illustrative examples, a probability of a tumor being present 238 in a subject 206 that provided a sample 204 can be determined by the computing system 214 by executing a model that corresponds to the following equation:

$$P ( cancer \vert region scores ) = \frac{1}{1 + {e}^{- {\sum }_{i} {w}_{i} ( score of region i ) + b}} , \qquad ( 2 )$$

where wᵢ is a weight of an individual classification region, the score of the region i is calculated using Equation (1), and b is a slope corresponding to a linear regression model. In at least some examples, the probability of a tumor being present 238 can be used to generate a status of tumor detected 232 or a status of tumor not detected 234. In one or more further illustrative examples, the computing system 214 can analyze the probability of a tumor being present 238 with respect to a threshold probability to determine a status of tumor detected 232 or a status of tumor not detected 234 for a sample 204. The computing system 214 can determine that a sample 204 corresponds to the status of tumor detected 232 in response to determining that a probability of a tumor being present 238 for the sample 204 is at least the threshold probability. Additionally, the computing system 214 can determine that a sample 204 corresponds to the status of tumor not detected 234 in response to determining that a probability of a tumor being present 238 for the sample 204 is less than the threshold probability.

In one or more additional examples, the computing system 214 can execute a model that determines a maximum mutant allele fraction (MAF). In various examples, the computing system 214 can execute a model using the maximum MAF value to determine tumor fraction 236 for a sample 204. In one or more illustrative examples, the computing system 214 can execute a model using the classification region metrics 226 to determine a logit transformed maximum MAF value that can then be used by the computing system 214 to estimate tumor fraction for a sample 204. In various examples, the computing system 214 can analyze maximum MAF values to determine a probability of cancer being present 238 in a subject 206 that provided a sample 204. In various examples, a Huber regression (Huber, P. J. 1964. “Robust Estimation of a Location Parameter.” Annals of Mathematical Statistics 35 (1): 73-101) can be performed to determine a maximum MAF value based on the classification region metrics 226.

In various examples, the model output 230 can also include a tumor tissue indication 240. The tumor tissue indication 240 can indicate one or more tissues from which cancer cells that produced genomic material detected in the sample 204 originate. In one or more examples, the tumor tissue indication 240 can correspond to one or more tissues of origin for cancer cells that produced genomic material detected in the sample 204. In these scenarios, the computing system 214 can generate multiple models with individual models corresponding to a given tissue type. The output from individual models can be analyzed to determine additional metrics that indicate a tissue from which cancer cells that produced genomic material detected in one or more samples originate. In at least some examples, the output for the individual models can indicate at least one of tumor fraction 236 or a probability of tumor being present 238. The computing system 214 can analyze the respective model outputs to determine the model having at least one of a greatest value for tumor fraction or a greatest probability of cancer being present. The computing system 214 can then generate a tumor tissue indication 240 that corresponds to the model having the greatest value for tumor fraction and/or a greatest probability of cancer being present.

For example, samples 204 can be obtained from subjects 206 in which different types of cancer are present. To illustrate, first samples can be obtained from a first group of subjects in which a first classification of cancer is present and second samples can be obtained from a second group of subjects in which a second classification of cancer is present. The sequencing data generated from the first samples can be analyzed by the computing system 214 to generate first metrics that correspond to classification regions for the first classification of cancer and the first metrics can be used to generate a first model that corresponds to the first classification of cancer. Additionally, the sequencing data generated from second samples can be analyzed by the computing system 214 to generate second metrics that correspond to classification regions for the first classification of cancer and the second metrics can be used to generate a second model that corresponds to the second classification of cancer. After the models have been trained, the computing system 214 can analyze sequencing data obtained from one or more additional subjects that were not included in the training subjects to determine classification region metrics for the one or more additional subjects. The classification region metrics can then be analyzed using the different tumor classification models to generate model outputs. The model outputs can be analyzed by the computing system to determine a model having greatest values for the respective model outputs and determine the tumor tissue classification that corresponds to the model.

In one or more additional illustrative implementations, the model output 230 can also indicate methylation status for one or more genomic regions of a reference sequence. For example, the computing system 214 can analyze the classification region metrics 226 to determine a methylation status of one or more promoter regions of a reference sequence. In various examples, the one or more promoter regions can include at least one promoter region that is related to the presence of a tumor in a subject. In one or more illustrative examples, the classification region metrics 226 can indicate a number of sequence representations having at least a threshold amount of methylation with respect to the one or more promoter regions. In these scenarios, the computing system 214 can determine that a promoter region is methylated in response to determining that the number of sequence representations having at least a threshold amount of molecules with a methylated cytosine in the promoter region is greater than a threshold number.

In still other illustrative implementations, the computing system 214 can analyze the sequencing data 212 to determine a quantitative measure that corresponds to a number of sequence representations that correspond to a promoter region and determine an additional quantitative measure that correspond to an additional number of sequence representations that correspond to a number of positive control regions. Normalized metrics can be determined based on the quantitative measure and the additional quantitative measure. The normalized metrics can be analyzed with respect to a threshold to determine a methylation status of the promoter region. In various examples, the threshold may be different for different promoter regions. The threshold can be determined using a cancer-free dataset and the threshold value is set such that false positive rates are no greater than 5%, no greater than 4%, no greater than 3%, no greater than 2%, or no greater than 1% and at a specificity of at least 80%, at least 90%, at least 95%, at least 98%, or at least 99%. In at least some examples, the computing system 214 can analyze the promoter region with regard to the threshold in situations where at least 4 sequence representations correspond to the promoter region, where at least 5 sequence representations correspond to the promoter region, where at least 6 sequence representations correspond to the promoter region, where at least 7 sequence representations correspond to the promoter region, where at least 8 sequence representations correspond to the promoter region, where at least 9 sequence representations correspond to the promoter region, where at least 10 sequence representations correspond to the promoter region, where at least 11 sequence representations correspond to the promoter region, or where at least 12 sequence representations correspond to the promoter region.

In one or more further illustrative examples, the computing system 214 can combine results from multiple models to determine the model output 230. For example, the computing system 214 can execute models with respect to one or more epigenetic signals, such as methylation of classification regions, to determine one or more first tumor metrics. With regard to methylation, the computing system 214 can execute both a classification model, such as a logistic regression model, that produced an indication of cancer being present in a subject providing a sample and an additional model that predicts tumor fraction for a sample. In one or more examples, the epigenetic signals can also correspond to fragment lengths of sequence representations generated from samples. In addition, the computing system 214 can execute one or more additional models with respect to genomic signals to generate further tumor metrics with respect to samples. In various examples, the genomic signals can correspond to the presence of one or more single nucleotide variants (SNVs) and/or the presence of insertions or deletions at one or more genomic regions of a reference sequence. In at least some examples, the computing system can include an integration system that combines tumor metrics generated by executing a number of models with regard to data corresponding to the genomic signals and the epigenetic signals to produce an aggregated tumor metric for a given sample. In some embodiments, using the quantitative measure obtained from the model output 230 can be analyzed with respect to a threshold. In situations where the quantitative measure obtained from the model output is at least the threshold value, the computing system 214 can determine that the indication of cancer is being present in the subject. In situations where the quantitative measure obtained from the model output is less than the threshold value, the computing system 214 can determine that the indication of cancer is being absent or not detected in the subject. In some embodiments, the threshold used to determine whether the indication of cancer is being present is calculated using a set of normal samples and is set at a particular value that provides high specificity.

In various additional implementations, the computing system 214 can determine methylation status of individual genomic regions. In one or more illustrative examples, the computing system 214 can determine methylation status of one or more promoter regions. In one or more examples, the sequencing data 212 can be analyzed to determine sequence representations that correspond to one or more genomic regions. For example, the sequencing data 212 can be analyzed to determine a number of sequence representations that correspond to one or more promoter regions. In at least some examples, the computing system 214 can determine a number of sequence representations that correspond to individual promoter regions that have at least a threshold amount of methylated cytosines.

For each genomic region and for an individual sample, the computing system 214 can determine a number of sequence representations that correspond to polynucleotide molecules having at least the threshold number of methylated cytosines in the genomic region. The computing system 214 can perform one or more normalization operations using the counts of polynucleotide molecules or sequence reads that correspond to the genomic region and have at least the threshold number of methylated cytosines to generate normalized metrics. To illustrate, the computing system 214 can divide the counts of polynucleotide molecules or reads that correspond to the genomic region and have at least the threshold number of methylated cytosines by the number of molecules or sequencing reads that correspond to a control region, such as a positive control region. In another instance, the computing system 214 can perform the normalized metrics by dividing the counts of polynucleotide molecules or reads that correspond to the genomic region and have at least the threshold number of methylated cytosines by the number of molecules or sequencing reads in a control dataset (i.e., the control dataset comprises of tumor not-detected samples) corresponding to the same genomic region and have at least the same threshold number of methylated cytosines.

The normalized metrics can be analyzed with respect to a threshold value. The threshold value can correspond to a given genomic region, such as a given promoter region. In various examples, the threshold value can be different for different promoter regions. In these scenarios, a first promoter region can have a first threshold value and a second promoter region can have a second threshold value. In situations where the normalization metric is at least the threshold value, the computing system 214 can determine that the genomic region has a first methylation status. In scenarios where the normalization metric is less than the threshold value, the computing system 214 can determine that the genomic region has a second methylation status. In one or more illustrative examples, the first methylation status can be labeled as “methylated” and the second methylation status can be labeled as “not methylated.”

The threshold value for a given genomic region can be determined based on training data obtained from samples of individuals in which cancer is not detected. In one or more examples, sequence representations obtained from the training samples can be analyzed to determine a z-score with respect to the number of polynucleotide molecules that correspond to the genomic region and that have at least the threshold amount of methylated cytosines. In one or more illustrative examples, the threshold value for a promoter region that is used to determine the normalization metrics for the promoter region can be derived from the z-score calculated based on the training samples with respect to the promoter region.

Although the illustrative example of FIG. 2 describes that models can be generated to determine a number of indicators with respect to the presence or absence of cancer in a given subject, in at least some additional examples, the sequencing data 212 can be analyzed by the computing system 214 to determine indicators of the presence of cancer without training specific models. In one or more examples, the computing system 214 can determine a tumor fraction value based on sequencing data 212 generated from one or more samples obtained from a single subject in which it is unknown whether or not cancer is present in the subject. In one or more examples, the computing system 214 can determine a change in the tumor fraction value based on sequencing data 212 generated from one or more samples obtained at two or more time points from a single subject. The change in the tumor fraction value can be used to monitor the subject's response to treatment. In one or more additional examples, a first sample can be obtained from a subject prior to or at onset of at least one administration of a treatment or a procedure related to cancer and one or more second samples can be obtained from the subject after at least one of administration of a treatment or a procedure related to cancer. In one or more illustrative examples, the one or more second samples can be obtained at least one week, at least two weeks, at least three weeks, at least four weeks, at least five weeks, at least six weeks, at least eight weeks, or at least ten weeks after administration of the treatment or procedure. In at least some examples, first sample and the second sample can be derived from at least one of a bodily fluid obtained from the subject or tissue obtained from the subject.

In one or more examples, one or more samples can be obtained from a given subject. The sequencing data 212 generated from the one or more samples can be analyzed by the computing system to determine quantitative measures for a number of classification regions. In one or more examples, the quantitative measures can correspond to an amount of sequence representations that have at least a threshold amount of overlap with one or more classification regions. In one or more additional examples, the quantitative measures can correspond to sequence representations having at least a threshold amount of methylated cytosines in CpG regions having at least a threshold amount of CG content. In various examples, the indication of cancer being present in the subject can include tumor fraction. In one or more additional examples, the indication of cancer being present in the subject can include mutant allele fraction. In at least some examples, the quantitative measures can correspond to a number of sequencing reads that correspond to a given classification region in relation to a total number of sequencing reads across a plurality of positive control regions. In one or more further examples, the indicators of cancer being present can be used to determine an output that corresponds to cancer being present or not being present in a given individual in response to analyzing the one or more indicators of cancer being present with respect to one or more thresholds. In one or more illustrative examples, tumor fraction determined from one or more samples obtained from a subject can be analyzed with respect to one or more thresholds. In instances where tumor fraction is greater than a threshold level, the computing system 214 can determine that the probability of cancer being present in the subject is at least 80%, at least 85%, at least 90%, at least 95%, or at least 99%. Further, in situations where multiple samples are obtained from a subject, first quantitative measures generated from a first sample obtained from the subject can be analyzed with respect to second quantitative measures generated from a second sample obtained from the subject. In at least some examples, differences between the first quantitative measures and the second quantitative measures can be analyzed to determine an indication of treatment response in the subject.

The quantitative measures used to determine an indication of cancer being present in a subject can be determined by analyzing quantitative measures of a subset of classification regions. In at least some examples, the subset of classification regions can be different for different subjects. In one or more illustrative examples, values of quantitative measures for a number of classification regions can be analyzed with respect to one another and ranked according to the magnitude of the value of the quantitative measures. In various examples, the classification regions for a given sample can be ranked in descending order from the one or more classification regions having the greatest value of a quantitative measure to the one or more classification region having the least value of the quantitative measure.

In various examples, after ranking the quantitative measures of the classification regions, quantitative measures that correspond to a group of the classification regions can be removed before determining the indication of cancer being present in the subject. For example, the group of classification regions that are not used to determine the indication of cancer being present in the subject can include the 1% of classification regions having the greatest quantitative measure values, the 2% of classification regions having the greatest quantitative measure values, 3% of classification regions having the greatest quantitative measure values, 4% of classification regions having the greatest quantitative measure values, 5% of classification regions having the greatest quantitative measure values, or the 6% of classification regions having the greatest quantitative measure values. In at least some examples, a number of classification regions having relatively high quantitative measure values can be excluded from the group of classification regions used to determine the indication of cancer being present in the subject because, in at least some cases, classification regions corresponding to quantitative measure values at or near the top of the ranked list can have non-tumor origins and/or be related to sequencing artifacts. Thus, by removing the quantitative measures that correspond to these classification regions from the analysis used to determine the indication of cancer being present in the subject, the accuracy with which the indication of cancer being present in the subject can increase.

In one or more examples, after determining the group of classification regions to be used to determine an indication of cancer being present in the subject, a subset of classification regions of the group can then be determined by identifying at least 10 classification regions of the group, at least 25 classification regions of the group, at least 50 classification regions of the group, at least 75 classification regions of the group, at least 100 classification regions of the group, at least 150 classification regions of the group, at least 200 classification regions of the group, at least 250 classification regions of the group, at least 300 classification regions of the group, at least 350 classification regions of the group, at least 400 classification regions of the group, at least 450 classification regions of the group, or at least 500 classification regions of the group having the greatest values for the respective quantitative measure.

In at least some examples, one or more statistical measures, such as at least one of mean, median, or mode, can be applied to the quantitative measures of the subset of the classification regions of the group to generate an initial indication of cancer being present in the subject. In various examples, the initial indication of cancer can be modified according to a scaling factor. The scaling factor can be applied to the initial indication of cancer being present in the subject because, in at least some scenarios, the positive control regions can have different amounts of methylated CpGs. For example, at least a portion of the positive control regions can have fully methylated CpGs while other positive control regions may not be fully methylated. Additionally, in various situations, some classification regions can correspond to a high value of an indication of cancer being present in subjects, such as 90% tumor fraction, 95% tumor fraction, 99% tumor fraction, or 100% tumor fraction, but nucleic acid molecules that correspond to these classification regions may not be fully methylated. To account for these cases, the scaling factor can be applied to the initial indication of cancer being present in the subject to provide a more accurate determination of the indication. In one or more illustrative examples, the scaling factor can be determined by analyzing indications of cancer being present in subjects determined using one or more techniques described herein in relation to additional data that corresponds to additional indications of cancer being present in subjects, such as validation data or other techniques that generate data orthogonal to the indications of tumors being present in subjects described herein.

In various examples, the classification regions used to determine the quantitative measures can correspond to classification regions that correspond to one or more portions of differentially methylated regions. In one or more examples, the differentially methylated regions can include promoter regions that correspond to one or more classifications of cancer. For example, the classification regions can be determined by analyzing a number of sequencing representations across a differentially methylated region. In these scenarios, one or more portions of the differentially methylated regions that overlap with at least at threshold number of sequencing representations can be included in the classification regions. In one or more examples, the quantitative measures of the one or more portions of the differentially methylated regions can be determined based on the molecule count distribution of the differentially methylated region. For example, the quantitative measures can be determined based on the molecule count within one or more peaks of the molecule distribution of the differentially methylated region. To illustrate, in various examples, the distribution of molecules across a differentially methylated region can indicate one or more peaks where greater amounts of molecules overlap with one or more subregions within the differentially methylated region. In various examples, the one or more genomic regions that correspond to the one or more subregions of the differentially methylated regions that correspond to the highest amounts of sequence representations for a sample can be defined as classification regions. In at least some examples, the distribution of sequence representations can have a peak that corresponds to a subregion of the differentially methylated region having a higher number of sequence representations than other subregions of the differentially methylated region. In these scenarios, the subregion can be identified as a classification region. By determining subregions of at least a portion of the differentially methylated regions used to determine the indication of cancer being present in the subject, the amount of computing resources and memory resources used to determine the indication of cancer being present in the subject can be decreased.

To illustrate, a classification region can include one or more portions of a differentially methylated region in which at least 50% of the sequencing representations obtained from a sample overlap, at least 55% of the sequencing representations obtained from a sample overlap, at least 60% of the sequencing representations obtained from a sample overlap, at least 65% of the sequencing representations obtained from a sample overlap, at least 70% of the sequencing representations obtained from a sample overlap, at least 75% of the sequencing representations obtained from a sample overlap, at least 80% of the sequencing representations obtained from a sample overlap, at least 85% of the sequencing representations obtained from a sample overlap, at least 90% of the sequencing representations obtained from a sample overlap, at least 95% of the sequencing representations obtained from a sample overlap, or at least 99% of the sequencing representations obtained from a sample overlap. In one or more illustrative examples, the one or more portions of the differentially methylated region that comprise a classification region can be contiguous with respect to a reference sequence.

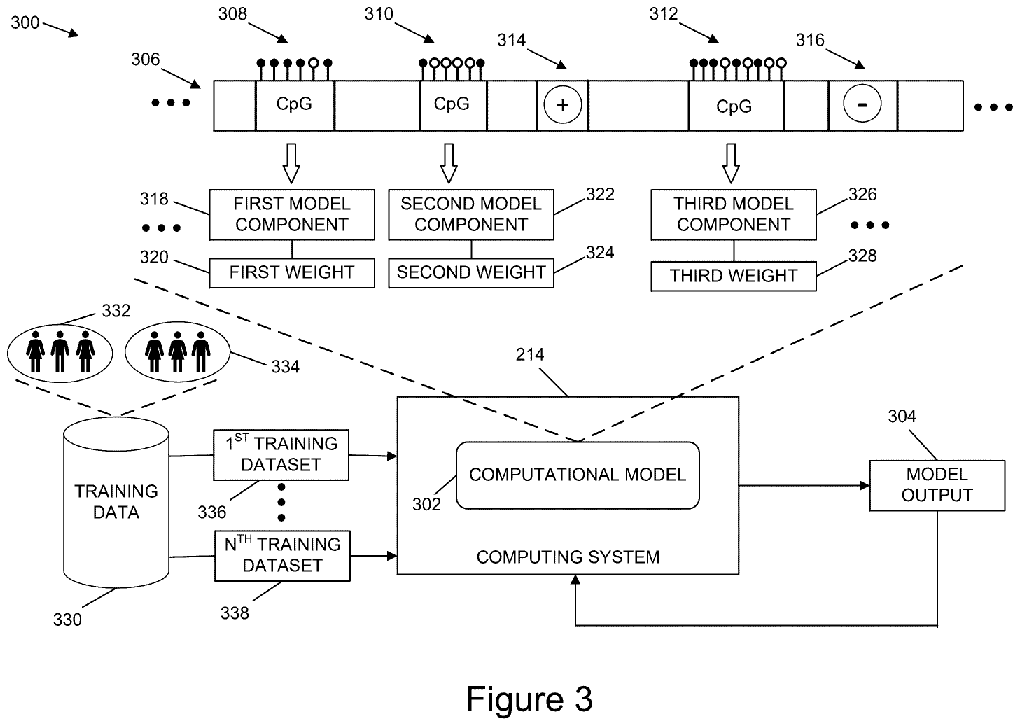

FIG. 3 is a diagrammatic representation of an example framework 300 to train a computational model 302 to determine one or more tumor metrics with respect to a sample, in accordance with one or more implementations. The framework 300 can include the computing system 214. The computing system 214 can execute the computational model 302 to generate one or more model outputs 304. In one or more examples, the computational model 302 can be a machine learning model. The model output 304 can include an indication corresponding to the presence or absence of a tumor in a subject that provided a sample. In one or more illustrative examples, the model output 304 can include a tumor fraction. In one or more additional illustrative examples, the model output 304 can include a probability of cancer being present in a subject. In one or more further illustrative examples, the model output can include an indication of cancer being present in a subject or an indication of cancer not being present in a subject. In still other illustrative examples, the model output 304 can indicate methylation status of one or more regions of nucleic acid molecules. To illustrate, the computing system 214 can execute the computational model 302 with respect to quantitative measures corresponding to a promoter region to determine an amount of methylation of the promoter region. In other illustrative examples, the model output 304 can include a tumor tissue indication of the sample.

The framework 300 can also include a sequence representation 306. In one or more examples, the sequence representation 306 can be generated based on analyzing nucleic acid molecules that are derived from a sample provided by a subject. The sequence representation 306 can include genomic regions having a number of nucleotides that correspond to a number of regions of interest. For example, the sequence representation 306 can include a sequence of nucleotides that corresponds to a first classification region 308. In addition, the sequence representation 306 can include a sequence of nucleotides that corresponds to a second classification region 310. Further, the sequence representation 306 can include a sequence of nucleotides that corresponds to a third classification region 312. In various examples, the first classification region 308, the second classification region 310, and the third classification region 312 of the sequence representation 306 can have differing amounts of methylated cytosines included in the respective classification regions 308, 310, 312. In one or more additional examples, the sequence representation 306 can include a sequence of nucleotides that corresponds to a positive control region 314 and a sequence of nucleotides that corresponds to a negative control region 316.

The computational model 302 can include a number of components that correspond to individual classification regions. In one or more examples, the components of the computational model 302 can have respective values that correspond to quantitative metrics of the respective classification regions. The quantitative metrics can indicate a number of sequence representations that correspond to the respective classification regions. In one or more examples, the computational model 302 can include a number of weights that are related to the respective components of the computational model 302. For example, the computational model 302 can include a first model component 318 that has a first weight 320. The first model component 318 can correspond to the first classification region 308. In addition, the computational model 302 can include a second model component 322 that has a second weight 324. The second model component 322 can correspond to the second classification region 310. In various examples, at least one of the first weight 320, the second weight 324, or the third weight 328 can be different from at least another one of the first weight 320, the second weight 324, or the third weight 328.

In one or more illustrative examples, a value for the first model component 318, the second model component 322, and the third model component 326 can be determined on a per sample basis. To illustrate, for different samples, the computational model 302 can determine different values for at least one of the first model component 318, the second model component 322, or the third model component 326. In various examples, the computing system 214 can determine first quantitative measures for the first classification region 308 based on sequencing data for a sample. The computing system 214 can execute the computational model 302 to determine a value for the first model component 318 based on the first quantitative measures. Additionally, the computing system 214 can determine second quantitative measures for the second classification region 310 based on sequencing data for the sample. The computing system 214 can execute the computational model 302 to determine a value for the second model component 322 based on the second quantitative measures. Further, the computing system 214 can determine third quantitative measures for the third classification region 312 based on sequencing data for the sample. The computing system 214 can execute the computational model 302 to determine a value for the third model component 326 based on the third quantitative measures. The first quantitative measures, the second quantitative measures, and the third quantitative measures can be determined based on numbers of sequence representations that have at least a threshold amount of methylation in CG regions that correspond to the first classification region 308, the second classification region 310, and the third classification region 312, respectively. In one or more additional illustrative examples, a value for the first weight 320, a value for the second weight 324, and a value for the third weight 328 can be determined on a per sample basis. For example, for different samples, the computational model 302 can determine different values for at least one of the first weight 320, the second weight 324, or the third weight 328.

In one or more examples, the computing system 214 can perform a training process to generate the computational model 302. In various examples, the training process can determine one or more features related to classification region metrics that can be used to determine the model output 304. Additionally, the training process can determine one or more parameters related to classification region metrics that can be used to determine the model output 304. For example, the training process can be used to determine the model components to include in the computational model 302 and the corresponding weights of the model components.

In the illustrative example of FIG. 3, the training process can be performed using training data 330. The training data 330 can include information obtained with respect to at least a first group of subjects 332 and information obtained with respect to at least a second group of subjects 334. In one or more examples, the first group of subjects 332 can include subjects in which a tumor is not detected and the second group of subjects 334 can include subjects in which a tumor is detected. In various examples, the training data 330 can include characteristics related to amounts of methylation of classification regions of the reference sequence 306 for the first group of subjects 332 and the second group of subjects 334. For example, the training data 330 can indicate quantitative measures corresponding to numbers of sequence representations that have at least a threshold level of methylation for the classification regions 308, 310, 312 for the first group of subjects 332 and the second group of subjects 334. The training data 330 can also include weights for model components based on an analysis of sequencing data of the first group of subjects 332 and the second group of subjects 334. In one or more illustrative examples, the training data 330 can include values for the first weight 320, values for the second weight 324, and values for the third weight 328 based on classification region metrics determined from sequencing data obtained from samples provided by the first group of subjects 332 and the second group of subjects 334.

The training data 330 can also include information corresponding to additional characteristics of the first group of subjects 332 and the second group of subjects 334. To illustrate, the training data 330 can include medical records information, medical history information, cancer treatment history information, demographic information, genomics information, one or more combinations thereof, and the like.

In one or more examples, the computing system 214 can train the computational model 302 to determine an indication related to one or more types of cancer being present in an individual. Additionally, in various examples, the computational model 302 can comprise multiple different models, such that the computational model 302 is an ensemble model. In these situations, the computing system 214 can perform one or more training processes with respect to individual models of the ensemble model. In one or more illustrative examples, the computational model 302 can include a number of individual models that each correspond to determining model outputs for individual genomic regions, such as genes or for a specified group of genes. For example, the computational model 302 can include a number of individual models to generate maximum MAF values for individual genes or for a specified groups of genes. In these scenarios, the training of the computational model 302 can have more constraints than other models used to determine indications of cancer being present in individuals because of the use of genomic information in the training process. As a result, in situations where the computational model 302 is training using genomic information, the accuracy of the output of the computational model 302 can be increased. Further, although the training of the computational model 302 can incorporate genomic information to determine maximum MAF values, the use of the computational model 302 to determine indications of cancer for test subjects after training can be performed without the use of genomic information and can be based on input vectors that correspond to quantitative measures determined from sequencing data obtained from the test subjects.

The computing system 214 can obtain a first training dataset 336 up to an Nth training dataset 338 to perform a training process to generate the computational model 302. In one or more examples, the first training dataset 336 can include a first portion of the training data 330 corresponding to the first group of subjects 332 and the second group of subjects 334 that is used to train the computational model 302 and the Nth training dataset 338 can include a second portion of the training data 330 corresponding to the first group of subjects 332 and the second group of subjects 334 as part of a validation process for the computational model 302. In various examples, the computational model 302 can be updated over time and undergo multiple training processes. In these scenarios, the first training dataset 336 can include a portion of the training data 330 for the first group of subjects 332 and the second group of subjects 334 that corresponds to a first period of time and the Nth training dataset 338 can include a portion of the training data 330 for the first group of subjects 332 and the second group of subjects 334 that corresponds to a second period of time.

In one or more examples, during the training process for the computational model 302, the computing system 214 can perform one or more optimization operations. In one or more illustrative examples, the computing system 214 can identify, during the training process for the computational model 302, one or more samples obtained from at least one of the first group of subjects 332 or the second group of subjects 334 that are outliers with respect to samples obtained from other subjects included in at least one of the first group of subjects 332 or the second group of subjects 334. To illustrate, the computing system 214 can determine that model output 304 generated for one or more subjects included in at least one of the first group of subjects 332 or the second group of subjects 334 has at least a threshold amount of difference with the model output 304 generated for one or more additional subjects included in at least one of the first group of subjects 332 or the second group of subjects 334. In one or more examples, the computing system 214 can identify at least one of one or more first subjects 332 or one or more second subjects 334 have model output 304 that is at least one standard deviation, at least 1.5 standard deviations, at least 2 standard deviations, at least 2.5 standard deviations, or at least 3 standard deviations different from a mean model output 304 determined for an additional group of at least one of the first group of subjects 332 or the second group of subjects 334. In various examples, the computing system 214 can apply a penalty to information generated from samples that correspond to subjects that are outliers with respect to information generated from samples that correspond to additional subjects.

In one or more additional examples, one or more optimization processes implemented by the computing system 214 in the training of the computational model 302 can correspond to a number of training cycles and/or a number of iterations for individual training cycles that are performed during the training process.

In one or more illustrative examples, the computing system 214 can perform at least 1000 iterations of a training process to generate the computational model 302, at least 3000 iterations of a training process to generate the computational model 302, at least 5000 iterations of a training process to generate the computational model 302, at least 8000 iterations of a training process to generate the computational model 302, at least 10,000 iterations of a training process to generate the computational model 302, at least 12,000 iterations of a training process to generate the computational model 302, or at least 15,000 iterations of a training process to generate the computational model 302. In various examples, the computing system 214 can end the training process before convergence of a loss function related to the computational model 302. In one or more examples, the number of iterations of the training process to produce the computation model 302 can correspond to a number of iterations of the training process performed before the training process is stopped and before the convergence of the loss function.

In one or more examples, a first stage of the training process implemented by the computing system 214 to generate the computational model 302 can include determining samples included in the training data 330 that include somatic mutations indicative one or more types of cancer in relation to samples included in the training data 330 that do not include somatic mutations indicative of the one or more types of cancer. The computing system 214 can then performing a training process for the computational model 302 using the samples of the training data 330 that include one or more somatic mutations indicative of the one or more types of cancer and using a number of samples obtained from subjects in which a tumor is not detected. In various examples, at least 100 iterations of the first stage of the training process can be performed.

Further, the training process performed by the computing system 214 can include a second stage that includes predicting values of tumor metrics of samples that do not include somatic mutations with respect to the one or more types of cancer. The computing system 214 can the perform at least 100 additional iterations of the second stage of the training process to generate the computational model 302. The second stage of the training process performed by the computing system 214 to generate the computational model 302 can also include training the computational model 302 using portions of the training data 330 corresponding to samples having somatic mutations indicative of the one or more types of cancer, using the predicted values of sample that do not include somatic mutations indicative of the one or more types of cancer, and portions of the training data 330 that correspond to samples obtained from subjects in which a tumor is not detected. In various examples, the second stage of the training process performed by the computing system 214 to generate the computational model 302 can be performed at least 2 additional times, at least 3 additional times, at least 4 additional times, at least 5 additional times, or at least 6 additional times. After the first stage of the training process and the second stage of the training process have been completed, the computing system 214 can perform a validation process for the computational model 302 using information obtained from different samples included in the training data 330.

In one or more illustrative examples, the computing system 214 can perform a training process for multiple computational models 302. In these scenarios, individual computational models 302 trained by the computing system 214 can correspond to different tissue types that are sources of genomic material obtained from subjects included in the training data 330. In one or more examples, the individual computational models 302 trained by the computing system 214 can correspond to different classification of cancer, such as colorectal cancer, lung cancer, pancreatic cancer, bladder cancer, breast cancer, liver cancer, skin cancer, or one or more additional classifications of cancer. In situations where the computing system 214 trains multiple computational models 302 that correspond to different classifications of cancer, the output from individual computational models 302 can be aggregated and analyzed by the computational system 214 to determine a tissue of origin for a subject.

In various examples, the individual computational models 302 that correspond to a given tissue from which genomic material included in samples is derived can have different model components. For example, a first computational model generated by the computing system 214 that corresponds to a first tissue type can have first model components that correspond to a first set of classification regions. In addition, a second computational model generated by the computing system 214 that corresponds to a second tissue type can have second model components that correspond to a second set of classification regions that has at least one classification region different from the first set of classification regions. Additionally, the weights for the individual components of the computational models that correspond to different tissue types can be different. That is, in situations where the first set of classification regions of the first computational model and the second set of classification regions of the second computational model have at least one classification region in common, the weights for the model component that corresponds to the at least one common classification region can be different in relation to the first computational model and the second computational model.

Additionally, one or more additional normalization processes can be performed by the computing system when generating the computational model 302. For example, in at least some scenarios, molecules treated with MBD can be partitioned differently across different samples. In one or more examples, molecules can be partitioned differently across different samples due to differences in the composition of reagents used to treat the molecules with MBD. In one or more additional examples, molecules can be partitioned differently across different samples due to at least one of equipment differences or process conditions used to treat the molecules with MBD.

To illustrate, for one or more first samples, treatment with MBD can cause first molecules having regions with first CG content to be separated into a first partition and second molecules having regions with second CG content to be separate into a second partition. In addition, for one or more second samples, treatment with MBD can cause third molecules having third CG content that is different from the first CG content to be separated into the first partition and fourth molecules having regions with fourth CG content that is different from the second CG content to be separated into the second partition. In various examples, the first molecules can be treated with MBD and separated into the first partition and the second molecules can be treated with MBD and separated into the second partition across a first cutoff range of CG content. Further, the third molecules can be treated with MBD and separated into the first partition and the fourth molecules can be treated with MBD and separated into the second partition across a second cutoff range of CG content that is different from the first cutoff range.

In one or more illustrative examples, the first cutoff range of CG content can include from 3-10 CpGs having methylated cytosines and the second cutoff range can include from 6-14 CpGs having methylated cytosines. In one or more additional illustrative examples, the first cutoff range of CG content can include from 4-9 CpGs having methylated cytosines and the second cutoff range can include from 7-13 CpGs having methylated cytosines. In one or more further illustrative examples, the first cutoff range of CG content can include from 5-8 CpGs having methylated cytosines and the second cutoff range can include from 8-12 CpGs. In still other illustrative examples, the first cutoff range of CG content can include 4-7 CpGs and the second cutoff range can include from 6-10 CpGs. In various examples, the first cutoff range of CG content and the second cutoff range of CG content can be used to determine the threshold amount of methylated cytosines used to determine at least one of training sequencing reads or testing sequencing reads. In at least some examples, the threshold amount of methylated cytosines can include a cutoff number that corresponds to a probability, such as at least about 80%, at least about 85%, at least about 90%, at least about 95%, or at least about 99% of individual molecules treated with MBD being separated into a given partition. In one or more examples, the threshold amount of methylated cytosines can correspond to 5 methylated cytosines, 6 methylated cytosines, 7 methylated cytosines, 8 methylated cytosines, 9 methylated cytosines, 10 methylated cytosines, 11 methylated cytosines, 12 methylated cytosines, 13 methylated cytosines, or 14 methylated cytosines.

In one or more examples, the computing system 214 can generate metrics for individual classification regions based on quantitative measures that are determined by analyzing a first number of sequencing reads to identify a first number of nucleic acid molecules having a first amount of CG content and by analyzing a second number of sequencing reads to identify a second number of nucleic acid molecules having a second amount of CG content. In at least some examples, the second number of nucleic acid molecules can be used to modify a metric determined using the first number of nucleic acid molecules to account for variations in the separation of molecules treated using MBD for different samples. In various examples, for individual classification regions, a first metric can be determined for a given sample by determining a first quantitative measure that corresponds to a number of molecules having a threshold amount of methylated cytosines and having a first amount of cytosine-guanine content in one or more partitions (for example, second partition 130 and/or third partition 134) that correspond to the individual classification region. In some embodiments, the first amount of CG content can be at least 5, at least 6, at least 7, at least 8, at least 9, at least 10, at least 15, at least 20, at least 25, or at least 30 CpGs in the nucleic acid molecules. In some embodiment, the first amount of CG content can be between 5-10, 5-15, 5-20, 5-30, 10-15, 10-20, 10-30, 15-20, 15-30 or 20-40 CpGs in the nucleic acid molecules

The first metric can also be determined for a given sample by determining a second quantitative measure that corresponds to a number of molecules having a threshold amount of methylated cytosines and having the first amount of cytosine-guanine content in one or more partitions (for example, second partition 130 and/or third partition 134) that correspond to a plurality of control regions (e.g., positive control regions). To illustrate, for an individual classification region, the first metric can be determined using the first quantitative measure for the individual classification region and the second quantitative measure that corresponds to the plurality of control regions.

The normalization process can also include determining, for a given sample, a second metric for the given sample by determining one or more additional quantitative measures based on a number of molecules in one or more partitions (e.g., second partition 130 and/or third partition 134) having at least the threshold amount of methylated cytosines and a second amount of cytosine-guanine content that correspond to the plurality of control regions, where the second amount of cytosine-guanine content is less than the first amount of cytosine-guanine content. In some embodiments, the second amount of CG content can be between 5-10, 5-15, 10-15, 10-20 or 15-20 CpGs in the nucleic acid molecules. In one or more examples, the plurality of control regions can be positive control regions and/or negative control regions. In one or more examples, the second metric can be determined using the additional quantitative measure and the second quantitative measure. In at least some examples, the second metric can be determined for a given sample by determining a ratio of the one or more additional quantitative measures with respect to the second quantitative measure. In one or more additional examples, the second metric can be determined for a given sample by determining the logarithm, such as the logarithm according to base 10, of a ratio of the one or more additional quantitative measures with respect to the second quantitative measure.

In one or more illustrative examples, the second metric for a given sample can include a combination of values, where individual values correspond to an additional quantitative measure based on a number of molecules having at least a threshold amount of methylated cytosines and a given number of CpGs for the plurality of control regions and the second quantitative measure. For example, a first additional quantitative measure can be determined based on a first number of molecules having at least the threshold amount of methylated cytosines in control regions having a first number of CpGs, such as 6, and a second additional quantitative measure can be determined based on a second number of molecules having at least the threshold amount of methylated cytosines in control regions having a second number of CpGs, such as 7. In at least some examples, more additional quantitative measures can be determined based on additional numbers of molecules having the threshold amount of methylated cytosines in control regions having additional numbers of CpGs, such as 8 CpGs, 9, CpGs, 10 CpGs, and the like up to an upper threshold of CpGs, such as 12 CpGs, 13 CpGs, or 14 CpGs. Ratios determined using the additional quantitative measures with respect to the second quantitative measures can be determined and summed to determine the second metric.

In various examples, a correlation factor can also be determined for individual classification regions in relation to different amounts of CpGs that can be used to determine the second metric. In one or more examples, the correlation factor can be modify the individual additional quantitative measures and then the modified individual additional quantitative measures can be aggregated to determine the second metric. In one or more additional examples, the first metric and the second metric can be combined to determine a normalized metric that corresponds to a given classification region. In one or more illustrative examples, the second metric can be subtracted from the first metric to determine the normalized metric.

In one or more additional illustrative examples, the correlation factor for a given classification region can be determined for each of a plurality of different amounts of cytosine-guanine content, such as a first correlation factor for 6 CpGs, a second correlation factor for 7 CpGs, a third correlation factor for 8 CpGs, and so forth up to a threshold amount of CG content. In at least some examples, the correlation factor can be determined by analyzing training data using one or more linear regression techniques. For example, the training data 330 can be fit to a linear regression model for individual classification regions to determine the correlation factor. In various examples, the fitting of at least a portion of the training data 330 to the linear regression model can be performed by aggregating the additional quantitative measures for a given classification region across a range of CG content, such a 6 CpGs, 7 CpGs, up to a threshold number of CpGs, and determining a mean quantitative measure.

In one or more examples, the normalized metrics can reduce variation of quantitative measures determined for individual samples. In at least some examples, the reduction in variation can result in increased accuracy of model outputs 304 in relation to at least some model outputs 304 determined without implementing the additional normalization process to determine the normalized metric.

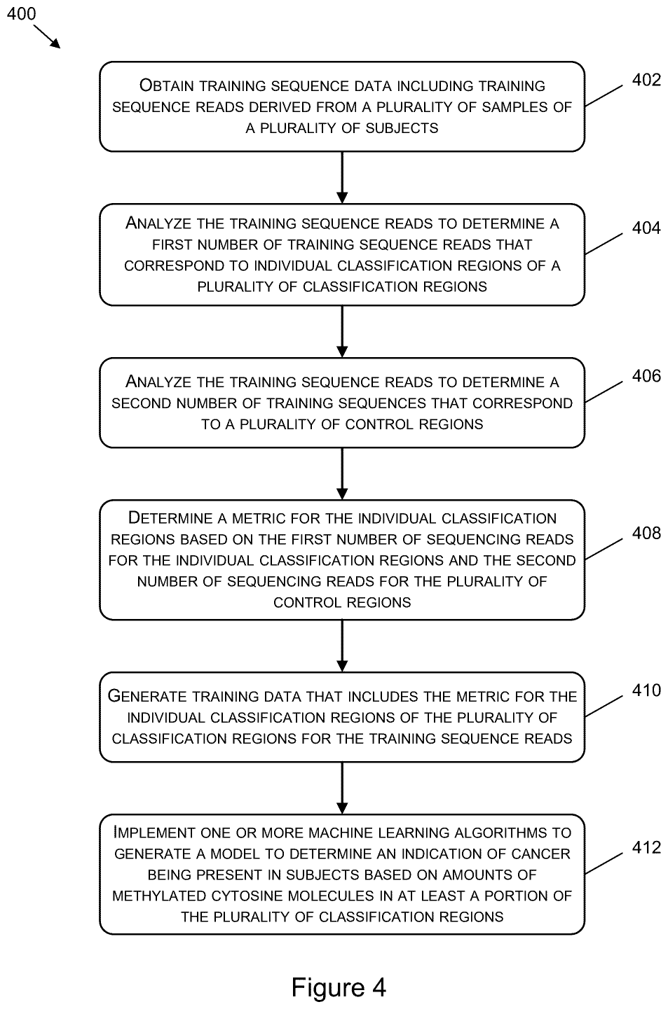

FIG. 4 is a flowchart of an example method 400 to determine tumor metrics in a subject based on levels of methylation of classification regions, according to one or more implementations. At operation 402, the method 400 can include obtaining training sequence data including training sequencing reads derived from a plurality of samples of a plurality of subjects. Individual training sequencing reads can include a nucleotide sequence corresponding to a fragment of a nucleic acid included in a sample of the plurality of samples. Individual training sequencing reads can have a threshold amount of molecules with a methylated cytosine included in regions of the nucleotide sequence having at least a threshold cytosine-guanine content. In one or more illustrative examples, the plurality of samples can include cell-free nucleic acids. In one or more examples, methylated cytosines can be determined using at least one of sodium bisulfite conversion and sequencing, Tet-assisted bisulfite sequencing (TAB-Seq), differential enzymatic cleavage, treatment with MSRE and/or MDRE, or MBD partitioning. In one or more additional examples, methylated cytosines can be determined using one or more single molecule sequencing methods, such as nanopore DNA sequencing or those described in Eid, J., et al. (2009) Real-time DNA sequencing from single polymerase molecules. Science, 323(5910), 133-138.

In one or more examples, the training process can include obtaining, by the computing system, testing sequence data from an additional subject that is not included in the plurality of subjects. The testing sequence data can include testing sequencing reads derived from a sample of the additional subject. Individual testing sequencing reads can include a nucleotide sequence corresponding to a fragment of a nucleic acid included in the additional sample. Additionally, individual testing sequencing reads can have at least the threshold amount of molecules with a methylated cytosine included in regions of the nucleotide sequence having at least the threshold cytosine-guanine content. Based on the additional sequence data, a model can be executed to determine the indication of cancer being present in the additional subject. The testing sequencing reads can then be analyzed to determine a first quantitative measure derived from the testing sequencing reads that correspond to the individual classification regions of the plurality of classification regions. Further, the testing sequencing reads can be analyzed to determine a second quantitative measure derived from the testing sequencing reads that correspond to the individual control regions the plurality of control regions. The metric can then be determined for the individual classification regions based on the first quantitative measure for the individual classification regions and the second quantitative measure for the plurality of control regions. Subsequently, an input vector can be generated that includes the metrics for the individual classification regions. The model can use the input vector to determine the indication of cancer being present in the additional subject.

In situations where the model is trained to determine an estimate of tumor fraction, the training sequencing reads can comprise a first portion of the training sequence data and a second portion of the training sequence data includes additional training sequencing reads that are different from the training sequencing reads. In these scenarios, at least one of the first portion of the training sequence data or the second portion of the training sequence data can be analyzed to determine an individual frequency of a plurality of variants present in individual samples of the plurality of samples. With respect to individual samples, a variant of the plurality of variants having a maximum frequency can then be determined that corresponds to the individual frequency having a greatest value among individual frequencies derived from an individual sample. In one or more illustrative examples, the maximum mutant allele frequency can be determined for individual samples. In various examples, individual measures of tumor fraction for the individual samples can then be determined based on the greatest value of the individual frequencies derived from the individual sample.

In at least some examples, the training process for the model can include one or more optimization operations. For example, the training process can include determining one or more additional weights of individual samples included in the training data based on the indication of cancer for the individual samples being within a threshold confidence level. In response to determining that the indication of cancer for an individual sample is outside of the threshold confidence level a penalty to can be applied to the individual sample during the training process.

The one or more training optimization operations can also include performing, using the one or more machine learning algorithms, one or more first iterations of the training process for the model using a portion of the training data. In addition, first output data for the model can be generated based on the one or more first iterations of the training process. The first output data can correspond to one or more first additional indications of cancer being present in first individual subjects of the plurality of subjects and the first individual subjects can correspond to the portion of the training data. Further, the training process can include combining the first output data and the training data to produce additional training data and performing one or more second iterations of the training process for the model using a portion of the additional training data. Second output data can then be generated for the model based on the one or more second iterations of the training process. The second output data can indicate one or more second additional indications of cancer being present in second individual subjects of the plurality of subjects where the second individual subjects corresponding to the portion of the additional training data. In one or more illustrative examples, the weights for the individual classification regions of the plurality of classification regions can be determined based on the first output data and the second output data.

Further, the training process can include determining that a number of indications of cancer are present that were determined during one or more iterations of the training process and have at least a threshold value for one or more samples included in the training data. In these scenarios, modifications to one or more weights of the model are not modified or are modified by a minimal amount. Additionally, an additional number of indications of cancer being present can be determined that were determined during the one or more iterations of the training process and are less than the threshold value for one or more additional samples included in the training data. In these scenarios, modifications to one or more additional weights of the model can be determined and the one or more additional weights are modified by more than the minimal amount.

In addition, at operation 404, the process 400 can include analyzing the training sequencing reads to determine a first quantitative measure derived from the training sequencing reads that corresponds to individual classification regions of a plurality of classification regions. In one or more examples, the first quantitative measure can be determined based on the number of training sequencing reads. In one or more additional examples, the first quantitative measure can be determined based on a number of polynucleotide molecules that correspond to the training sequencing reads. At least a portion of the individual classification regions of the plurality of classification regions corresponding to genomic regions of a reference genome that have the threshold amount of molecules with a methylated cytosine in subjects in which cancer is detected and that have at least the threshold cytosine-guanine content. In various examples, the plurality of classification regions can correspond to genomic regions in which at least one mutation occurs in patients in which cancer is detected. Additionally, the plurality of classification regions can correspond to a first plurality of classification regions for a first cancer type and the model can be generated for a second cancer type based on a second plurality of classification regions that are different from the first plurality of classification regions.

At operation 406, the process 400 can include analyzing the training sequencing reads to determine a second quantitative measure derived from the training sequencing reads that correspond to a plurality of control regions. In one or more examples, the second quantitative measure can be determined based on the number of training sequencing reads. In one or more additional examples, the second quantitative measure can be determined based on a number of polynucleotide molecules that correspond to the training sequencing reads. Individual control regions of the plurality of control regions corresponding to additional genomic regions of the reference genome that have at least the threshold cytosine-guanine content. Additionally, the individual control regions can have at least the threshold amount of molecules with a methylated cytosine in subjects in which cancer is detected and in additional subjects in which cancer is not detected

Further, at operation 408, the process 400 can include determining a metric for the individual classification regions of the plurality of classification regions based on the first quantitative measure for the individual classification regions and the second quantitative measure for the plurality of control regions. In one or more examples, the metric for the individual classification regions is determined based on a scaling factor and an error correction factor. In one or more illustrative examples, the scaling factor can include a logarithmic function and the error correction factor can include a pseudocount.

At operation 410, the process 400 can include generating, by the computing device, training data that includes the metric for the individual classification regions of the plurality of classification regions for the training sequence reads. In implementations where the indication of cancer is tumor fraction, the training data can include the individual measures of tumor fraction for the individual samples of the plurality of samples and the model can be executed with respect to individual measures of tumor fraction for the individual samples of the plurality of samples.

The process 400 can also include, at operation 412, implementing, using the training data, one or more machine learning algorithms to generate a model to determine an indication of cancer being present in subjects based on amounts of molecules with methylated cytosines in at least a portion of the plurality of classification regions. The model can determine weights for individual classification regions of the plurality of classification regions and at least a portion of the weights of the individual classification regions can be different from one another. In various examples, the one or more machine learning algorithms can include one or more classification algorithms and the indication of cancer being present corresponds to a probability of cancer being present in the additional subject. In one or more additional examples, the one or more machine learning algorithms include one or more regression algorithms and the indicator corresponds to an estimate of tumor fraction of the additional sample. In one or more illustrative examples, a limit of detection for the model to determine tumor fraction of samples can be no greater than 0.01% given 95% sensitivity, no greater than 0.05% given 95% sensitivity, no greater than 0.1% given 95% sensitivity, no greater than 0.15% given 95% sensitivity, no greater than 0.2% given 95% sensitivity, no greater than 0.25% given 95% sensitivity, or no greater than 0.3% given 95% sensitivity.

In various examples, the sequence reads provided to the model during the training process or after the training process have at least a threshold amount of methylated cytosines in classification regions. The sequence reads that satisfy the methylation levels can be produced, at least in party, using one or more molecule separation processes. The molecule separation processes can include combining a plurality of nucleic acids derived from at least one of blood or tissue of a subject with a solution including an amount of methyl binding domain (MBD) proteins to produce a nucleic acid-MBD protein solution. A plurality of washes can then be performed of the nucleic acid-MBD protein solution with a salt solution to produce a number of nucleic acid fractions. Individual nucleic acid fractions can have a threshold number of molecules with a methylated cytosine in regions of the plurality of nucleic acids having at least the threshold cytosine-guanine content. In one or more illustrative examples, a wash of the plurality of washes can be performed with a solution having a concentration of sodium chloride (NaCl) and can produce a nucleic acid fraction of the number of nucleic acid fractions having a range of binding strengths to MBD proteins.

In one or more examples, a first nucleic acid fraction can be determined is associated with a first partition of a plurality of partitions of nucleic acids. The first partition corresponding to a first range of binding strengths to MBD proteins. Further, a first molecular barcode can be attached to nucleic acids of the first nucleic acid fraction. The first molecular barcode can be associated with the first partition. In addition, a second nucleic acid fraction can be determined that is associated with a second partition of the plurality of partitions of nucleic acids. The second partition can correspond to a second range of binding strengths to MBD proteins different from the first range of binding strengths to MBD proteins. A second molecular barcode can be attached to nucleic acids of the second nucleic acid fraction. The second molecular barcode being associated with the second partition.

In one or more additional examples, at least a portion of the number of nucleic acid fractions can be combined with an amount of restriction enzyme that cleaves molecules with one or more unmethylated cytosines to produce at least a portion of the plurality of samples used to produce the sequencing reads. In these scenarios, the threshold amount of molecules with a methylated cytosine corresponds to a minimum frequency of molecules with a methylated cytosine within a region having at least the threshold cytosine-guanine content. In one or more further examples, at least a portion of the number of nucleic acid fractions are combined with an amount of a restriction enzyme that cleaves molecules with a methylated cytosine to produce at least a portion of the plurality of samples used to produce the sequencing reads. In these situations, the threshold amount of molecules with a methylated cytosine corresponds to a maximum frequency of molecules with a methylated cytosine within a region having at least the threshold cytosine-guanine content.

### Exemplary Methods

### A. Determining an Indication of Cancer in a Sample

In some embodiments, methods disclosed herein comprise sequencing cfDNA from a sample and determining methylation levels for a plurality of target regions comprising DNA sequences that are differentially methylated regions and control regions. In some embodiments, methods disclosed herein comprise capturing at least an epigenetic target region set from cfDNA or a subsample thereof, comprising contacting the cfDNA or subsample thereof with target-specific probes specific for the at least one epigenetic target region set, determining methylation levels for the target regions and determining whether an indication of cancer is present or not in sample obtained from a subject. In any of these embodiments, methylation levels/status of nucleic acid molecules can be determined using one or more of the methods comprising partitioning of molecules using a binding agent that recognized a modified cytosine, MSRE/MDRE digestion, methylation-sensitive conversion such as bisulfite conversion, direct detection during sequencing, or any other suitable approach. Various approaches are described herein.

In some embodiments, methods disclosed herein comprise steps of partitioning a sample comprising DNA by contacting the DNA with an agent that recognizes a modified cytosine in the DNA, sequencing the DNA, and determining quantitative measure of the nucleic acids in a plurality of regions.

In one or more aspects, a method includes obtaining, by a computing system having one or more hardware processors and memory, training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The method also includes analyzing, by the computing system, the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The method also includes analyzing, by the computing system, the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The method also includes determining, by the computing system, a metric for the individual classification regions of the plurality of classification regions based on the first quantitative measure for the individual classification regions and the second quantitative measure for the plurality of control regions. The method also includes generating, by the computing device, training data that includes the metric for the individual classification regions of the plurality of classification regions for the training sequence reads from samples of training subjects. The method also includes implementing, by the computing system and using the training data, one or more machine learning algorithms to generate a model to determine an indication of cancer being present in subjects based on amounts of methylated cytosines in at least a portion of the plurality of classification regions, the model including weights for individual classification regions of the plurality of classification regions and at least a portion of the weights of the individual classification regions being different from one another.

In one or more aspects, a computing system includes: one or more hardware processors; and one or more non-transitory computer-readable storage media including computer-readable instructions that, when executed by the one or more hardware processors, cause the one or more hardware processors to perform operations comprising: obtaining training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The operations also include analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The operations also include analyzing the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The operations also include determining a metric for the individual classification regions of the plurality of classification regions based on the first quantitative measure for the individual classification regions and the second quantitative measure for the plurality of control regions. The operations also include generating training data that includes the metric for the individual classification regions of the plurality of classification regions for the training sequence reads from samples of training subjects. The operations also include implementing, using the training data, one or more machine learning algorithms to generate a model to determine an indication of cancer being present in subjects based on amounts of methylated cytosines in at least a portion of the plurality of classification regions, the model including weights for individual classification regions of the plurality of classification regions and at least a portion of the weights of the individual classification regions being different from one another.

In one or more aspects, one or more computer-readable storage media comprise computer-readable instructions that, when executed by one or more processors of a computing system, cause the computing system to perform operations comprising: obtaining training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The operations also include analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The operations also include analyzing the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The operations also include determining a metric for the individual classification regions of the plurality of classification regions based on the first quantitative measure for the individual classification regions and the second quantitative measure for the plurality of control regions. The operations also include generating training data that includes the metric for the individual classification regions of the plurality of classification regions for the training sequence reads from samples of training subjects. The operations also include implementing, using the training data, one or more machine learning algorithms to generate a model to determine an indication of cancer being present in subjects based on amounts of methylated cytosines in at least a portion of the plurality of classification regions, the model including weights for individual classification regions of the plurality of classification regions and at least a portion of the weights of the individual classification regions being different from one another.

In one or more aspects, a method includes obtaining, by a computing system having one or more hardware processors and memory, sequencing reads derived from a sample obtained from a subject, where individual sequencing reads include a nucleotide sequence corresponding to a fragment of a nucleic acid included in the sample and correspond to molecules having a threshold amount of methylated cytosines included in regions of the nucleotide sequence having at least a threshold cytosine-guanine content. The method may also include determining, by the computing system, a first quantitative measure derived from the sequencing reads that corresponds to individual classification regions of a plurality of classification regions, where at least a portion of the individual classification regions of the plurality of classification regions correspond to genomic regions of a reference genome that have the threshold amount of methylated cytosines in subjects in which cancer is detected and that have at least the threshold cytosine-guanine content. The method may also include analyzing, by the computing system, the sequencing reads to determine a second quantitative measure derived from the sequencing reads that correspond to a plurality of control regions, where individual control regions of the plurality of control regions correspond to additional genomic regions of the reference genome that have at least the threshold cytosine-guanine content and that have at least the threshold amount of methylated cytosines in subjects in which cancer is detected and in additional subjects in which cancer is not detected. The method may also include determining, by the computing system, a plurality of metrics with individual metrics of the plurality of metrics corresponding to individual classification regions of the plurality of classification regions based on the first quantitative measure for the individual classification regions and the second quantitative measure for the plurality of control regions. The method may also include determining, by the computing system, an indication of cancer being present in the subject based on at least a portion of the plurality of metrics.

In one or more aspects, a computing system comprises: a processor; and a memory storing instructions that, when executed by the processor, configure the computing system to: obtain sequencing reads derived from a sample obtained from a subject, individual sequencing reads including a nucleotide sequence corresponding to a fragment of a nucleic acid included in the sample and corresponding to molecules having a threshold amount of methylated cytosines included in regions of the nucleotide sequence having at least a threshold cytosine-guanine content. The computing system may also determine a first quantitative measure derived from the sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The computing system may also analyze the sequencing reads to determine a second quantitative measure derived from the sequencing reads that correspond to a plurality of control regions, with individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The computing system may also determine a plurality of metrics with individual metrics of the plurality of metrics corresponding to individual classification regions of the plurality of classification regions based on the first quantitative measure for the individual classification regions and the second quantitative measure for the plurality of control regions. The computing system may also determine an indication of cancer being present in the subject based on at least a portion of the plurality of metrics.

A computing system comprising: a processor; and a memory storing instructions that, when executed by the processor, configure the system to: obtain testing sequence data from a subject, the testing sequence data including testing sequencing reads derived from a sample of the subject, individual testing sequencing reads including a nucleotide sequence corresponding to a fragment of a nucleic acid included in the additional sample and individual testing sequencing reads corresponding to molecules having a threshold amount of methylated cytosines included in regions of the nucleotide sequence having at least the threshold cytosine-guanine content. The computing system may also analyze the testing sequencing reads to determine a first quantitative measure derived from the testing sequencing reads that correspond to individual classification regions of a plurality of classification regions at least a portion of the individual classification regions of the plurality of classification regions corresponding to genomic regions of a reference genome that have the threshold amount of methylated cytosines in subjects in which cancer is detected and that have at least the threshold cytosine-guanine content. The computing system may also analyze the testing sequencing reads to determine a second quantitative measure derived from the testing sequencing reads that correspond to individual control regions a plurality of control regions, with individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The computing system may also determine a metric for the individual classification regions based on the first quantitative measure for the individual classification regions and the second quantitative measure for the plurality of control regions. The computing system may also generate an input vector that includes the metrics for the individual classification regions. The computing system may also determine an indication of cancer being present in the subject by providing the input vector to a model that implements one or more machine learning techniques to generate indications of cancer being present in subjects, with the model including weights for individual classification regions of the plurality of classification regions and at least a portion of the weights of the individual classification regions being different from one another.

B. Partitioning the Sample into a Plurality of Subsamples

In some embodiments described herein, different forms of DNA (e.g., hypermethylated and hypomethylated DNA) are physically partitioned based on one or more characteristics of the DNA. This approach can be used to determine, for example, whether certain sites or regions are hypermethylated or hypomethylated. Partitioning can be performed before attaching adapters to DNA molecules in the sample, e.g., so as to facilitate including partition tags in the adapters. Partition tags can be used to identify which partition a molecule was found in. Following partitioning (and attachment of adapters if applicable), further steps such as amplification, target capture, and sequencing may be performed.

Methylation profiling can involve determining methylation patterns across different regions of the genome. For example, after partitioning molecules based on extent of methylation (e.g., relative number of methylated nucleobases per molecule) and further steps as discussed above including sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of particular immune cell types, such as rare immune cell types, tumor cells or cells of a type that does not normally contribute to the DNA sample being analyzed (such as cfDNA).

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

In some embodiments, partitioning is on the basis of one or more characteristics such as methylation. Molecules can be sorted according to other characteristics, such as sequence length, nucleosome binding, sequence mismatch, immunoprecipitation, and/or proteins that bind to DNA, using appropriate techniques as part of data analysis or partitioning as applicable. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by the procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

The binding of partitioning agents to particular nucleic acids and the partitioning of the nucleic acids into subsamples may occur to a certain extent or may occur in an essentially binary manner. In some instances, nucleic acids comprising a greater proportion of a certain modification bind to the agent at a greater extent than nucleic acids comprising a lesser proportion of the modification. Similarly, the partitioning may produce subsamples comprising greater and lesser proportions of nucleic acids comprising a certain modification. Alternatively, the partitioning may produce subsamples comprising essentially all or none of the nucleic acids comprising the modification. In all instances, various levels of modifications may be sequentially eluted from the partitioning agent.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner®Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, the partitioning of the sample into a plurality of subsamples is performed by contacting the nucleic acids with an antibody that recognizes a modified nucleobase in the DNA, which may be is a modified cytosine or a product of the procedure that affects the first nucleobase in the DNA differently from the second nucleobase in the DNA of the sample. In some embodiments, the modified nucleobase is 5mC. In some embodiments, the modified nucleobase is 5caC. In some embodiments, the modified nucleobase is dihydrouracil (DHU). In some embodiments, the antibody that recognizes a modified nucleobase in the DNA is used to partition single-stranded DNA.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, an MBD binds to 5mC and 5hmC. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, sample DNA (e.g., between 5 and 200 ng) is mixed with methyl binding domain (MBD) buffer and magnetic beads conjugated with MBD proteins and incubated overnight. Methylated DNA (hypermethylated DNA) binds the MBD protein on the magnetic beads during this incubation. Non-methylated (hypomethylated DNA) or less methylated DNA (intermediately methylated) is washed away from the beads with buffers containing increasing concentrations of salt. For example, one, two, or more fractions containing non-methylated, hypomethylated, and/or intermediately methylated DNA may be obtained from such washes. Finally, a high salt buffer is used to elute the heavily methylated DNA (hypermethylated DNA) from the MBD protein. In some embodiments, these washes result in three partitions (hypomethylated partition, intermediately methylated fraction and hypermethylated partition) of DNA having increasing levels of methylation.

In some embodiments, partitioning procedures may result in imperfect sorting of DNA molecules among the subsamples. For example, a minority of the molecules in an unmethylated or hypomethylated subsample may be highly modified (e.g., hypermethylated), and/or a minority of the molecules in a hypermethylated subsample may be unmodified or mostly unmodified (e.g., unmethylated or mostly unmethylated). Such molecules are considered nonspecifically partitioned.

In some embodiments, nonspecifically partitioned molecules are removed using a methylation-dependent nuclease, e.g., a methylation dependent restriction enzyme (MDRE), digesting/cleaving the DNA where the restriction enzyme (RE) recognition site contains a methylated nucleotide but not cleaving the DNA where the restriction enzyme (RE) recognition site contains an unmethylated nucleotide. In some embodiments, nonspecifically partitioned molecules are removed using a methylation sensitive nuclease, e.g., a methylation sensitive restriction enzyme (MSRE), digesting/cleaving the DNA where the restriction enzyme (RE) recognition site contains an unmethylated nucleotide but not cleaving the DNA where the restriction enzyme (RE) recognition site contains a methylated nucleotide. For example, in some embodiments, a hypomethylated subsample is contacted with a methylation-dependent nuclease, such as a methylation-dependent restriction enzyme, thereby degrading nonspecifically partitioned DNA, e.g., methylated DNA, in the subsample. Alternatively, or in addition, a hypermethylated subsample is contacted with a methylation-sensitive endonuclease, such as a methylation-sensitive restriction enzyme, thereby degrading nonspecifically partitioned DNA in the subsample.

Degradation of nonspecifically partitioned DNA in one or more partitioned subsamples may improve the performance of methods that rely on accurate partitioning of DNA on the basis of a cytosine modification. For example, such degradation may provide improved sensitivity and/or simplify downstream analyses. In some embodiments, partitioning DNA on the basis of a modification, such as methylation, then removing nonspecifically partitioned DNA using MDREs and/or MSREs as described herein provides improved efficiency and/or cost over DNA analysis methods comprising procedures that affect a first nucleobase differently from a second nucleobase, such as bisulfite sequencing or bisulfite conversion.

In some embodiments, one or more nucleases are used to degrade nonspecifically partitioned DNA molecules. In some embodiments, a subsample is contacted with a plurality of nucleases. The subsample may be contacted with the nucleases sequentially or simultaneously. Simultaneous use of nucleases may be advantageous when the nucleases are active under similar conditions (e.g., buffer composition) to avoid unnecessary sample manipulation. Contacting a subsample with more than one methylation-dependent restriction enzyme can more completely degrade nonspecifically partitioned hypermethylated DNA. Contacting a subsample with more than one methylation-sensitive restriction enzyme can more completely degrade nonspecifically partitioned hypomethylated and/or unmethylated DNA.

In some embodiments, a methylation-dependent nuclease comprises one or more of MspJI, LpnPI, FspEI, or McrBC. In some embodiments, at least two methylation-dependent nucleases are used. In some embodiments, at least three methylation-dependent nucleases are used.

In some embodiments, a methylation-sensitive nuclease comprises one or more of AatII, AccII, AciI, Aor13HI, Aor15HI, BspT1041, BssHII, BstUI, Cfr101, ClaI, Cpol, Eco521, HaeII, HapII, HhaI, Hin6I, HpaII, HpyCH4IV, MluI, MspI, NaeI, NotI, NruI, NsbI, PmaCI, Psp14061, PvuI, SacII, SaII, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, HpaII and Hin6I.

In some embodiments, the partitions of DNA are desalted and concentrated in preparation for enzymatic steps of library preparation.

### C. Adapter Ligation

In some embodiments, adapters are added to the DNA. This may be done concurrently with an amplification procedure, e.g., by providing the adapters in a 5′ portion of a primer (where PCR is used, this can be referred to as library prep-PCR or LP-PCR). In some embodiments, adapters are added by other approaches, such as ligation. In some such methods, prior to partitioning or prior to capturing, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

Preferably, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA is partitioned, comprising contacting the DNA with an agent that preferentially binds to nucleic acids bearing an epigenetic modification. The nucleic acids are partitioned into at least two subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in. In some embodiments, the nucleic acids are linked at both ends to Y-shaped adapters including primer binding sites and tags. The molecules are amplified.

### D. Tagging

“Tagging” DNA molecules is a procedure in which a tag is attached to or associated with the DNA molecules. Tags can be molecules, such as nucleic acids, containing information that indicates a feature of the molecule with which the tag is associated. For example, molecules can bear a sample tag (which distinguishes molecules in one sample from those in a different sample) or a molecular tag/molecular barcode/barcode (which distinguishes different molecules from one another (in both unique and non-unique tagging scenarios). For methods that involve a partitioning step, a partition tag (which distinguishes molecules in one partition from those in a different partition) may be included. In some embodiments, adapters added to DNA molecules comprise tags. In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member.

In some embodiments, two or more partitions, e.g., each partition, is/are differentially tagged. Tags can be used to label the individual polynucleotide population partitions so as to correlate the tag (or tags) with a specific partition. Alternatively, tags can be used in embodiments that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations). In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In some embodiments, the partition tags can serve as identifiers of the source partition and the molecule, i.e., different partitions are tagged with different sets of molecular tags, e.g., comprised of a pair of barcodes. In this way, the one or more molecular barcodes attached to the molecule indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded.

Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

### E. Enriching/Capturing Step; Amplification

Methods disclosed herein can comprise capturing DNA, such as cfDNA target regions. In some embodiments, the capturing comprises contacting the DNA with probes (e.g., oligonucleotides) specific for the target regions. Enrichment or capture may be performed on any sample or subsample described herein using any suitable approach known in the art.

In some embodiments, enrichment or capture is performed after attachment of adapters to sample molecules. In some embodiments, enrichment or capture is performed after a partitioning step. In some embodiments, enrichment or capture is performed after an amplification step. In some embodiments, sample molecules are partitioned, then adapters are attached, then sample molecules are amplified, and then the amplified molecules are subjected to enrichment or capture. The enriched or captured molecules may then be subjected to another amplification and then sequenced.

In some embodiments, the probes specific for the target regions comprise a capture moiety that facilitates the enrichment or capture of the DNA hybridized to the probes. In some embodiments, the capture moiety is biotin. In some such embodiments, streptavidin attached to a solid support, such as magnetic beads, is used to bind to the biotin. Nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification comprises use of a PCR primer that anneals to a sequence within an adapter and a PCR primer that anneals to a sequence within a probe annealed to the target region of the DNA.

In some embodiments, the methods herein comprise enriching for or capturing DNA comprising epigenetic and/or sequence-variable target regions. Such regions may be captured from an aliquot of a sample (e.g., a sample that has undergone attachment of adapters and amplification), while the step of partitioning the DNA with an agent that recognizes a modified cytosine, such as methyl cytosine, is performed on a separate aliquot of the sample. Enriching for or capturing DNA comprising epigenetic and/or sequence-variable target regions may comprise contacting the DNA with a first or second set of target-specific probes. Such target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from the first subsample or the second subsample, e.g., the first subsample and the second subsample. In some embodiments, the subsamples are differentially tagged (e.g., as described herein) and then pooled before undergoing capture. Exemplary methods for capturing DNA comprising epigenetic and/or sequence-variable target regions can be found in, e.g., WO 2020/160414, which is hereby incorporated by reference.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, methods described herein comprise capturing a plurality of sets of target regions of cfDNA obtained from a subject. The target regions may comprise differences depending on whether they originated from a tumor or from healthy cells or from a certain cell type. The capturing step produces a captured set of cfDNA molecules. In some embodiments, cfDNA molecules corresponding to a sequence-variable target region set are captured at a greater capture yield in the captured set of cfDNA molecules than cfDNA molecules corresponding to an epigenetic target region set. In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set. For additional discussion of capturing steps, capture yields, and related aspects, see WO2020/160414, which is incorporated herein by reference for all purposes.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In some embodiments, the DNA is amplified. In some embodiments, amplification is performed before the capturing step. In some embodiments, amplification is performed after the capturing step. In some embodiments, amplification is performed before and after the capturing step. In various embodiments, the methods further comprise sequencing the captured DNA, e.g., to different degrees of sequencing depth for the epigenetic and sequence-variable target region sets, consistent with the discussion herein.

In some embodiments, a capturing step is performed with probes for a sequence-variable target region set and probes for an epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, a capturing step is performed with a sequence-variable target region probe set in a first vessel and with an epigenetic target region probe set in a second vessel, or a contacting step is performed with a sequence-variable target region probe set at a first time and a first vessel and an epigenetic target region probe set at a second time before or after the first time. This approach allows for preparation of separate first and second compositions comprising captured DNA corresponding to a sequence-variable target region set and captured DNA corresponding to an epigenetic target region set. The compositions can be processed separately as desired (e.g., to partition based on methylation as described herein) and pooled in appropriate proportions to provide material for further processing and analysis such as sequencing.

In some embodiments, adapters are included in the DNA as described herein. In some embodiments, tags, which may be or include barcodes, are included in the DNA. In some embodiments, such tags are included in adapters. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described herein. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed herein, which can be combined to the extent practicable with any of these embodiments.

F. Procedures that Affect a First Nucleobase in the DNA Differently from a Second Nucleobase in the DNA or Methylation-Sensitive Conversion Methods

In some embodiments, methods disclosed herein comprise a step of subjecting DNA to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases comprises mC and the other comprises hmC.

In some embodiments, the procedure that affects a first nucleobase in the DNA differently from a second nucleobase in the DNA comprises bisulfite conversion. Treatment with bisulfite converts unmodified cytosine and certain modified cytosines (e.g. 5-formyl cytosine (fC) or 5-carboxylcytosine (caC)) to uracil whereas other modified cytosines (e.g., 5-methylcytosine, 5-hydroxylmethylcystosine) are not converted. Performing bisulfite conversion can facilitate identifying positions containing mC or hmC using the sequence reads. For an exemplary description of bisulfite conversion, see, e.g., Moss et al., Nat Commun. 2018; 9:5068. In some embodiments, the procedure that affects a first nucleobase in the DNA differently from a second nucleobase in the DNA comprises oxidative bisulfite (Ox-BS) conversion. Performing Ox-BS conversion can facilitate identifying positions containing mC using the sequence reads. For an exemplary description of oxidative bisulfite conversion, see, e.g., Booth et al., Science 2012; 336:934-937.

In some embodiments, the procedure that affects a first nucleobase in the DNA differently from a second nucleobase in the DNA comprises Tet-assisted bisulfite (TAB) conversion. For example, as described in Yu et al., Cell 2012; 149:1368-80, b-glucosyl transferase can be used to protect hmC (forming 5-glucosylhydroxymethylcytosine (ghmC)), then a TET protein such as mTetI can be used to convert mC to caC, and then bisulfite treatment can be used to convert C and caC to U while ghmC remains unaffected. Thus, when TAB conversion is used, the first nucleobase comprises one or more of unmodified cytosine, fC, caC, mC, or other cytosine forms affected by bisulfite, and the second nucleobase comprises hmC. Performing TAB conversion can facilitate identifying positions containing hmC using the sequence reads.

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. See, e.g., Liu et al., Nature Biotechnology 2019; 37:424-429 (e.g., at Supplementary FIG. 1 and Supplementary Note 7). Performing TAP conversion can facilitate identifying positions containing unmodified C using the sequence reads. This procedure encompasses Tet-assisted pyridine borane sequencing (TAPS), described in further detail in Liu et al. 2019, supra.

Alternatively, protection of hmC (e.g., using bOT) can be combined with Tet-assisted conversion with a substituted borane reducing agent. Performing such TAPSP conversion can facilitate distinguishing positions containing unmodified C or hmC on the one hand from positions containing mC using the sequence reads. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA comprises APOBEC-coupled epigenetic (ACE) conversion. Performing ACE conversion can facilitate distinguishing positions containing hmC from positions containing mC or unmodified C using the sequence reads. For an exemplary description of ACE conversion, see, e.g., Schutsky et al., Nature Biotechnology 2018; 36:1083-1090.

In some embodiments, procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI [0, available at www.biorxiv.org/content/10.1101/2019.12.20.884692vl.

In some embodiments, procedure that affects a first nucleobase in the DNA differently from a second nucleobase in the DNA comprises the methods involving direct methylation sequencing (DM-Seq) for detecting 5mC. Exemplary methods and compositions related to DM-Seq can be found in, e.g., WO2023/288222 and WO2021/236778, which are hereby incorporated by reference.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

### G. Captured Set; Target Regions

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naive lymphocytes or myeloid cells at different stages of differentiation.

In some embodiments, a captured epigenetic target region set captured from a sample or first subsample comprises hypermethylation target regions. In some embodiments, the hypermethylation target regions are differentially or exclusively hypermethylated in one cell type or in one immune cell type, or in one immune cell type within a cluster. In some embodiments, the hypermethylation target regions are hypermethylated to an extent that is distinguishably higher or exclusively present in one cell type or one immune cell type or one immune cell type within a cluster. Such hypermethylation target regions may be hypermethylated in other cell types but not to the extent observed in the one cell type. In some embodiments, the hypermethylation target regions show lower methylation in healthy cfDNA than in at least one other tissue type.

In some embodiments, a captured epigenetic target region set captured from a sample or second subsample comprises hypomethylation target regions. In some embodiments, the hypomethylation target regions are exclusively hypomethylated in one cell type or in one immune cell type or in one immune cell type within a cluster. In some embodiments, the hypomethylation target regions are hypomethylated to an extent that is exclusively present in one cell type or one immune cell type or in one immune cell type within a cluster.

Such hypomethylation target regions may be hypomethylated in other cell types but not to the extent observed in the one cell type. In some embodiments, the hypomethylation target regions show higher methylation in healthy cfDNA than in at least one other tissue type. Without wishing to be bound by any particular theory, in an individual with cancer, proliferating or activated immune cells (and potentially also cancer cells) may shed more DNA into the bloodstream than immune cells in a healthy individual (and healthy cells of the same tissue type, respectively). As such, the distribution of cell type and/or tissue of origin of cfDNA may change upon carcinogenesis. For example, the distribution of immune cell type of origin may change in a subject having cancer, precancer, infection, transplant rejection, or other disease or disorder directly or indirectly affecting the immune system. The status of epigenetic target regions of certain immune cell types likewise may change in a subject having such a disease relative to a healthy subject or relative to the same subject prior to having the disease or disorder. Thus, variations in hypermethylation and/or hypomethylation can be an indicator of disease. For example, an increase in the level of hypermethylation target regions and/or hypomethylation target regions in a subsample following a partitioning step can be an indicator of the presence (or recurrence, depending on the history of the subject) of cancer.

Exemplary hypermethylation target regions and hypomethylation target regions useful for distinguishing between various cell types, including but not limited to immune cell types, have been identified by analyzing DNA obtained from various cell types via whole genome bisulfite sequencing, as described, e.g., in Stunnenberg, H. G. et. al., “The International Human Epigenome Consortium: A Blueprint for Scientific Collaboration and Discovery,” Cell 167, 1145 (2016) (doi.org/10.1186/sl3059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set.

Where DNA (e.g., a sample or subsample) has been subjected to a procedure such as bisulfite conversion, treatment with a deaminase, or any of the other such procedures mentioned herein that alter the base-pairing specificity of certain bases, enrichment or capture may use oligonucleotides (e.g., primers or probes) specific for the altered or unaltered sequence, as desired.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section

### 1. Epigenetic Target Region Set

In some embodiments, an epigenetic target region set may comprise one or more types of target regions likely to differentiate DNA from different immune cell types and other non-immune cell types and/or to differentiate neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein.

In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

a. Hypermethylation Target Regions

In some embodiments, the epigenetic target region set comprises one or more hypermethylation target regions. In some embodiments, hypermethylation target regions are exclusively hypermethylated in one immune cell type or hypermethylated to a greater extent in one immune cell type than in any other immune cell type or than in any other immune cell type within the same immune cell cluster. In some such embodiments, hypermethylation target regions indicate the levels of particular immune cell types from which the DNA originated, including rare immune cell types such as activated B cells (including memory B cells and plasma cells), activated T cells (including regulatory T cells (Tregs), CD4 effector memory T cells, CD4 central memory T cells, CD8 effector memory T cells, and CD8 central memory T cells), and natural killer (NK) cells. Methylation patterns of hypermethylation target regions that are useful for deconvoluting immune cell types may further change in certain disease states, such as cancer. Thus, in some embodiments, hypermethylation target regions that are useful for deconvoluting immune cell types are also useful for determining the likelihood that the subject from which the sample was obtained has cancer or precancer. In some such embodiments, hypermethylation target regions are useful for determining whether levels of particular immune cell types are abnormal and whether such abnormal levels are likely related to the presence of cancer or precancer, or if they are related to a different disease or condition other than cancer or precancer.

In some embodiments, certain hypermethylation target regions exhibit an increase in the level of observed methylation, e.g., are hypermethylated, in DNA produced by neoplastic cells, such as tumor or cancer cells. Detection of such hypermethylation target regions, e.g., in conjunction with detection of hypermethylation target regions indicative of immune cell types, may further increase the specificity and/or sensitivity of methods described herein. In some embodiments, such increases in observed methylation in hypermethylated target regions indicate an increased likelihood that a sample (e.g., of cfDNA) was obtained from a subject having cancer. For example, hypermethylation of promoters of tumor suppressor genes has been observed repeatedly. See, e.g., Kang et ah, Genome Biol. 18:53 (2017) and references cited therein. In another example, as discussed above, hypermethylation target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation target regions. In some embodiments, hypermethylation target regions useful for determining the likelihood that a subject has cancer are different than the hypermethylation target regions useful for determining the levels of particular immune cell types. In some embodiments, at least some of the hypermethylation target regions useful for determining the likelihood that a subject has cancer are the same as the hypermethylation target regions useful for determining the levels of particular immune cell types.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation target regions based on colorectal cancer (CRC) studies is provided in Table 1. Many of these genes likely have relevance to cancers beyond colorectal cancer; for example, TP53 is widely recognized as a critically important tumor suppressor and hypermethylation-based inactivation of this gene may be a common oncogenic mechanism.

TABLE 1
Exemplary Hypermethylation Target
Regions based on CRC studies.

| | Additional | |
|---|---|---|
| | Gene | |
| Gene Name | Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, the hypermethylation target regions comprise a plurality of loci listed in Table 1, e.g., at least 10%, 20%, 30%, 40%, 50%, 60%, 70%, 80%, 90%, or 100% of the loci listed in Table 1. For example, for each locus included as a target region, there may be one or more probes with a hybridization site that binds between the transcription start site and the stop codon (the last stop codon for genes that are alternatively spliced) of the gene, or in the promoter region of the gene. In some embodiments, the one or more probes bind within 300 bp of the transcription start site of a gene in Table 1, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5 (5): 492-504 (2016); Skvortsova et al., Br. J. Cancer. 94 (10): 1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation target regions based on lung cancer studies is provided in Table 2. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 1 and 2, indicating generality.

TABLE 2
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 2 may be combined with any of the embodiments described above concerning target regions identified in Table 1. In some embodiments, the hypermethylation target regions comprise a plurality of loci listed in Table 1 or Table 2, e.g., at least 10%, 20%, 30%, 40%, 50%, 60%, 70%, 80%, 90%, or 100% of the loci listed in Table 1 or Table 2.

In some embodiments, the hypermethylation target regions comprise regions of one or more genes listed in Table 2b, e.g. at least 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1050,

1000, 1100, 1150 or 1200 genes listed in Table 3. Hypermethylation of these genes can be useful for detecting contributions from immune cells to a DNA sample. In some embodiments, the hypermethylation target regions comprise regions of a plurality of genes listed in Table 2b, e.g., at least 10%, 20%, 30%, 40%, 50%, 60%, 70%, 80%, 90%, or 100% of the genes listed in Table 3. In some embodiments, the hypermethylation target regions comprise regions of all of the genes listed in Table 3.

TABLE 3
Exemplary genes comprising exemplary hypermethylation target regions
GENES

| | | | | | | |
|---|---|---|---|---|---|---|
| CCL5 | RSAD2 | DBNDD1 | CD80 | CD2 | ITPK1 | PRR5 |
| CD19 | ISG15 | SNRPB | CEACAM4 | G0S2 | JAG1 | PTGS2 |
| CD37 | MMP3 | MNAT1 | SH2D1A | MT1G | MMRN2 | PTPN11 |
| CD3D | MARCKSL1 | NUP35 | SAA2-SAA4 | CARD16 | NFIL3 | PVRIG |
| CD3E | IGF2R | TCEB1 | TMEM150B | MT1H | PDGFB | RELA |
| CD3G | LAMB1 | HSPB11 | PYHIN1 | SERPINA1 | PGPEP1 | RICTOR |
| CD3Z | SPARC | C19orf48 | CCL7 | ACP5 | RPL7A | RIPK2 |
| CD79A | FN1 | ID3 | PDCD1 | CXCL5 | SERPINB5 | SPP1 |
| CD79B | ITGA4 | IPO4 | BNC1 | CD74 | SERPINH1 | STAT4 |
| CD8A | SMO | FARSB | ICOS | C1QB | STC1 | TNFRSF14 |
| CD8B1 | MMP19 | EIF4G1 | ITK | HLA-DRB5 | TNFAIP6 | TNFRSF25 |
| IGHG3 | ITGB8 | SKA1 | CLEC4E | FADS3 | VCAN | TNFSF9 |
| IGJ | ITGA5 | MFSD11 | GZMK | IL15RA | VEGFA | TRAT1 |
| IGLC1 | NID1 | PLAUR | UBASH3A | MFI2 | VEGFB | TSLP |
| CD14 | TIMP1 | MARVELD2 | CRTAM | IL411 | VEGFC | VTCN1 |
| LCK | SEMA3F | MCM3 | EOMES | HLA-A | ATF3 | CCL13 |
| LTB | RHOQ | DHFR | P2RY10 | TM4SF19 | BATF3 | CCL14 |
| MS4A1 | CTNNB1 | RNF41 | NCF1 | MMP9 | CD1C | CCL18 |
| CORO1A | MMP2 | ID2 | CARD17 | ACY3 | CD36 | CCL19 |
| MNDA | SERPINE1 | H2AFZ | ZBP1 | KRT14 | CD4 | CCL20 |
| CCRL2 | EPHB2 | CDK2 | TNFRSF9 | MT1X | CDC20 | CCL21 |
| SLC7A7 | COL16A1 | NCLN | PDCD1LG2 | MT1M | CTSW | CCL22 |
| HLA-DMA | EPHA2 | ZWILCH | IL12RB1 | IFITM1 | CXCL1 | CCL3/L1 |
| FYB | TNC | DYNLT1 | LILRA6 | IFI35 | DTX3L | CSF1 |
| RNASE6 | JUP | C16orf61 | CXCR3 | BST2 | HLA-DMB | CSF2 |
| TLR2 | ITGA3 | SLC25A40 | SLAMF1 | CD8B | HLA-DOA | CSF2RB |
| CTSC | TCF7L2 | RHOC | SLAMF6 | BATF2 | HLA-DOB | CSF3R |
| LILRB4 | COL3A1 | CCT5 | RHOH | CTSZ | HLA-DPA1 | CX3CL1 |
| PSCDBP | CDH6 | PDIA4 | GPR114 | HAPLN3 | HLA-DPB1 | CX3CR1 |
| CTSS | WNT2B | SNRPA | SLA2 | TAP2 | HLA-DQA1 | CXCL12 |
| RASSF4 | ADAM9 | RBM14 | IL18RAP | ODF3B | HLA-DQA2 | CXCL14 |
| MSN | DSP | PDLIM7 | GPR171 | SQRDL | IRF8 | CXCL16 |
| CYBB | HSPG2 | PITPNC1 | SCIMP | DOK2 | KIF2C | CXCL2 |
| LAPTM5 | ARHGAP1 | TPM3 | P2RY13 | UBE2L6 | KIR2DL3 | CXCL3 |
| DOCK2 | ITGB5 | CORO1C | LAX1 | SLC15A3 | KIR3DL1 | CXCL6 |
| FCGR1A | IGFBP5 | ERLIN1 | AOAH | SECTM1 | KIR3DL2 | CXCL8 |
| EVI2B | ARHGDIA | PAICS | CXCL11 | GAL | KLRD1 | CXCR2 |
| ADCY7 | LRP1 | TPRKB | CD38 | CXCL13 | MRC1 | GNG4 |
| CD48 | IGFBP2 | SKA2 | GRAP2 | RAC2 | PSMB10 | IL 10 |
| ARHGAP15 | CTNNA1 | MYBL1 | WDFY4 | IL1R2 | PSMB5 | IL11 |
| ARRB2 | LRRC17 | SH3BP5L | LTA | LRFN4 | PSMB8 | IL11RA |
| SYK | MMP14 | BRCA2 | GZMH | CLEC4A | PSMC4 | IL 12RB2 |
| BTK | NEO1 | SAR1A | PTPN22 | SLAMF8 | THBD | IL 16 |
| TNFAIP3 | EFNA5 | POLR3K | IRF4 | SH2D2A | TNF | IL 1A |
| FCGR2A | ITGB3 | MRPS28 | GNLY | CDC25B | TRIM21 | IL1B |
| VSIG4 | EPHB3 | NUP107 | CCR5 | PSME2 | UBA7 | IL1RN |
| FPRL2 | CD44 | TUBG1 | SNX20 | GNGT2 | UBB | IL22RA1 |
| IL10RA | IGFBP4 | PNN | LILRB5 | TYROBP | UBE2C | IL24 |
| IFI16 | TNFRSF1A | FAM167A | RGS18 | AIF1 | ULBP2 | IL33 |
| ITGB2 | RAC1 | RFC3 | EMR1 | WARS | VHL | IL34 |
| IL7R | PXN | MYL6 | TIGIT | CD300A | AKT1 | IL6 |
| TBXAS1 | PLAT | MCM7 | SIGLEC7 | C1QC | APC | IL6R |
| FMNL1 | COL8A1 | MAGOHB | GPR65 | ARPC1B | BAD | JAK2 |
| FLI1 | WNT8B | FAM89B | TNFRSF17 | CIITA | BAX | JAK3 |
| RASSF2 | IGFBP3 | TOMM40 | ADAMDEC1 | HAVCR2 | BBC3 | PF4 |
| LYZ | RHOA | CDCA4 | HK3 | SPI | BCL2 | PIK3R5 |
| CD163 | EPHB4 | MT3 | LILRB2 | MIR3123 | BCL2L1 | SHC2 |
| CD97 | MMP1 | MTHFD1 | CTLA4 | ACAA2 | BCL6B | STAT2 |
| CCL2 | PAK1 | PSMD12 | SIGLEC12 | KRTAP19-8 | BID | STAT3 |
| FCGR2B | MTA1 | MYBL2 | CXorf21 | LPCAT1 | BIRC3 | CBLC |
| MERTK | THBS2 | CKLF | SPIB | CARTPT | BIRC5 | CD47 |
| CD84 | CSPG2 | NRIP3 | CLEC4G | ARHGAP26 | BLM | CNTFR |
| CD53 | MMP17 | EZR | CD300C | RPA3 | CASP3 | GHR |
| CD86 | CD59 | C12orf24 | CD300E | MIR129-1 | CASP8 | GZMM |
| HMHA1 | DVL3 | GPLD1 | LILRB1 | FAM150A | CASP9 | IFITM2 |
| CTSL | RHOB | SRM | LAG3 | KCNB2 | CDH1 | IRF9 |
| EVI2A | COL6A3 | RAB3B | HMGA2 | TERF1 | FADD | JAK1 |
| TNFRSF1B | NOTCH2 | NLN | VNN1 | KCNK9 | HMGB1 | KLRB1 |
| CXCR4 | BSG | MT1F | IL2RA | PTK2 | LY96 | KLRK1 |
| LCP1 | MMP11 | TNFRSF12A | NCR3 | GPR20 | RIPK1 | LIF |
| SAMHD1 | COL1A2 | TPI1 | CCR8 | TSNARE1 | RIPK3 | PRLR |
| CPVL | ZYX | HAS2 | TFEC | MIR4475 | ROCK1 | SIRPA |
| HLA-DRB1 | RND3 | APOO | CCR2 | IGFBPL1 | TICAM1 | SPRY4 |
| C13orf18 | THBS1 | FBXO41 | STAP1 | LOC100129316 | TLR3 | DDB2 |
| GIMAP4 | RHOG | MRPL37 | IDO1 | QRFP | TLR4 | ERCC3 |
| SAMSN1 | ICAM1 | GSTCD | MYO1G | CAMSAP1 | TNFRSF10B | FANCA |
| PLCG2 | LAMA4 | SDC1 | SIT1 | GPR37L1 | TNFSF10 | MGMT |
| OSBPL3 | DVL1 | WDR54 | CD209 | CCDC85C | TP53 | NEIL1 |
| RUNX3 | PAK2 | RNF138 | LILRA5 | GACAT2 | BNIP3 | PARP4 |
| FCGR3A | COL6A1 | APITD1 | SIGLEC1 | LINC01443 | DEPTOR | POLR2A |
| AMPD3 | FGD1 | RMND5B | UBD | CDH2 | HIF1A | TNKS |
| MYO1F | CEP78 | ENO1 | SPN | MIR4436A | HRAS | UBE2T |
| CECR1 | LSM3 | MAP3K8 | CXCL9 | INSM1 | KRAS | XCL1 |
| LYN | LRRC40 | TMEM130 | CD70 | LOC101929625 | MAP3K7 | XCL2 |
| MPP1 | STK17A | SNX17 | GZMB | COL18A1 | MAPK10 | ARID1A |
| LRMP | RPN1 | KRR1 | SIGLEC10 | OPA1-AS1 | MTOR | BRD3 |
| FGL2 | JUNB | TAGLN | CD300LF | C9 | NRAS | BRD4 |
| NCKAP1L | NUP85 | PA2G4 | CCL8 | MIR585 | PIK3CA | DNMT1 |
| HCLS1 | FLNC | RUVBL1 | PTPN7 | MAD1L1 | PIK3CD | HDAC11 |
| SELL | HMGN2 | SNRPD1 | CCL4 | CPA2 | PIK3R1 | HDAC3 |
| CASP1 | RPP40 | LOXL2 | CMKLR1 | LOC100130298 | PIK3R2 | HDAC4 |
| SELPLG | UQCR10 | POLE2 | ICAM3 | C8orf34 | PRKAA2 | HDAC5 |
| CD33 | AIMP2 | MAPRE1 | SIRPG | OC90 | PRKACB | HELLS |
| GPNMB | CHEK1 | IMP4 | NLRP3 | TRAPPC9 | PTEN | HMGA1 |
| NCF2 | VTA1 | EMP2 | MARCO | MED27 | RPS6KB1 | KAT2B |
| FNBP1 | EXOSC8 | PSMD2 | CCL4L1 | MLH1 | RPTOR | MAP3K12 |
| IL18 | CENPO | MET | CD180 | MSH2 | ANLN | BMP2 |
| B2M | PNO1 | IFRD2 | SIGLEC9 | MSH6 | ATM | GAS1 |
| SP140 | SLC16A1 | LMNB2 | KRT6B | PMS2 | CCNA1 | GLI1 |
| FCER1G | WDR77 | PLOD2 | NFAM1 | BRCA1 | CCNB1 | IHH |
| LCP2 | UBE2J1 | NCEH1 | GPR84 | TERT | CCND1 | PRKX |
| LY86 | NOP16 | NME1 | KLK5 | EphA2 | CCND3 | WNT10A |
| LAIR1 | NUDT1 | STRA13 | CCL23 | cGAS | CCNO | WNT11 |
| IFI30 | SMC2 | ACTL6A | ZBED2 | STING | CDC25C | WNT2 |
| TNFSF13B | SLC25A5 | DLEU1 | CD28 | CD8 | CDK6 | WNT3A |
| LST1 | NUPL1 | SNRPA1 | CEACAM21 | PDL2 | CDKN1A | WNT4 |
| FGR | DLEU2 | CBX1 | PRF1 | ASXL3 | CDKN1C | WNT5A |
| NPL | PDAP1 | LYAR | CSF3 | SPEDF | CDKN2A | WNT5B |
| PLEK | CCBL2 | PTPLB | KIAA0226L | PSMA | CDKN2B | WNT7B |
| PTPRC | COX17 | PFN1 | CXCR6 | GPC3 | CENPF | ALDOA |
| GNPTAB | BCCIP | CENPJ | CCL24 | RAD51 | CEP55 | EGF |
| SLC1A3 | PLG | COTL1 | ARHGAP9 | ACVR1 | EXO1 | EGFR |
| HCK | RGS8 | SPRYD7 | LILRB3 | APLNR | H2AFX | ERBB2 |
| NPC2 | SNRPC | USPL1 | VNN2 | CDC27 | MELK | HK1 |
| C3AR1 | PLK4 | MRPL12 | ZAP70 | CDK4 | MKI67 | HK2 |
| PIK3CG | NUTF2 | ADAMTS1 | TNFSF8 | HLA-C | MYC | IFNGR1 |
| DAPK1 | LSM4 | GLRX3 | HEPHL1 | HLA-E | NBN | IFNGR2 |
| ALOX5AP | SMS | WSB2 | CCL3 | HLA-G | POLD1 | LDHA |
| CSF1R | EBNA1BP2 | MRPS16 | CLEC10A | IL1R | PRKCA | NOS2 |
| CUGBP2 | C13orf27 | DCLRE1B | CCL3L3 | IL2RB | RAD50 | PDK1 |
| APOE | VDAC1 | MKKS | CCR4 | INFGR1 | RAD51C | PFKFB3 |
| APOC1 | PSMD14 | C3orf26 | ITGAL | INFGR2 | RB1 | SLC2A1 |
| CD52 | MYCBP | CPEB4 | SAA2 | IRF1 | RBL2 | TFRC |
| LHFPL2 | SMURF2 | SPAG17 | GZMA | IRF2 | RRM2 | CD276 |
| C1orf54 | GNG11 | MLF1IP | BCL2A1 | KIR2DS2 | TYMS | CD6 |
| IKZF1 | F3 | UAP1 | RNASE2 | KIR2DS3 | ADORA2A | CDH5 |
| SH2B3 | BRIP1 | COQ2 | SAA1 | PIAS4 | BTLA | CLEC14A |
| WIPF1 | HNRNPA2B1 | WDHD1 | NKG7 | PTPN2 | CD247 | CLEC5A |
| IFI44 | DCK | DCBLD2 | MT2A | SERPINB3 | CD27 | CLEC7A |
| IFI44L | ALKBH7 | KIAA0090 | FAM26F | SERPINB4 | CD274 | CLECL1 |
| DDX58 | HN1L | SAR1B | PSMB9 | SPOP | CD40 | ICAM2 |
| IF16 | TPM1 | PSMA7 | TYMP | TAPBP | CD40LG | ICAM5 |
| IFI27 | HYLS1 | PSMC3 | CD7 | TGFBR1 | CD69 | ITGA1 |
| IFIT2 | HAUS1 | COPS6 | SYT12 | CD58 | CHUK | ITGA2 |
| IFIT1 | NUP93 | DUT | S100A7 | ANGPT1 | DPP4 | ITGAE |
| IFIT3 | SNRPE | PPIH | HLA-F | ANGPT2 | EGR1 | ITGAM |
| CXCL10 | ITGA6 | PHF19 | MT1E | ANGPTL4 | FYN | ITGAX |
| MX1 | CENPN | TPM2 | ISG20 | CCND2 | ICOSLG | NCAM1 |
| OAS1 | C11orf24 | MCTS1 | C1QA | CCNE1 | IKBKB | NECTIN1 |
| OAS2 | GGH | EIF4EBP1 | TNFRSF18 | CES3 | IKBKG | PECAM1 |
| OAS3 | PFKP | HNRNPR | IL32 | DLL4 | IL15 | PVR |
| HERC5 | FARSA | IFNG | SOCS1 | E2F3 | IL17A | SELE |
| SAMD9 | EIF2AK1 | TLR8 | HLA-DQB1 | EDN1 | IL18R1 | SELP |
| HERC6 | CENPW | FASLG | IFITM3 | EZH2 | IL2 | THY1 |
| DDX60 | TUBA4A | KIR2DL4 | PI3 | FGF18 | IL4 | VCAM1 |
| RTP4 | TRA2B | IL21R | HCST | FGFR1 | LY9 | EIF2AK2 |
| IFIH1 | UMPS | GBP5 | SUSD3 | FLT1 | NECTIN2 | FLNB |
| STAT1 | MRTO4 | ZNF683 | HLA-B | FSTL3 | NFATC2 | GBP1 |
| TAP1 | NUDT15 | PLA2G2D | IL2RG | HEY1 | NFKB1 | GBP2 |
| OASL | PGM2 | TBX21 | HLA-DRA | ITGAV | NFKBIA | GBP4 |
| TPSD1 | GATA2 | SERPINF1 | FCER1A | DAPP1 | CCR7 | EREG |
| SLC18A2 | HPGDS | PLAC8 | CD1A | C100RF54 | XCR1 | CD81 |
| IL1RL1 | TCF4 | JCHAIN | AXL | ANPEP | MTSS1 | PRDM1 |
| VWA5A | MZB1 | SLC15A4 | CD1E | CST7 | MSA4A7 | ABL2 |
| TPSAB2 | IL3RA | ITM2C | PKIB | FLT3 | LILRA1 | SDC4 |
| IFNA1 | PDGFA | MMP7 | CRABP2 | TNFRSF4 | FZD9 | PNOC |
| IFNAR1 | PDGFRB | NID2 | DAB2 | TNFSF12 | GPC4 | HLA-DR |
| IRF3 | PLA1A | RELN | FCAR | TNFSF13 | SFRP1 | LIN |
| IRF5 | PLA2G2A | AQP9 | FCN1 | TNFSF18 | SFRP4 | CD11b |
| IRF7 | RASAL1 | CEBPB | FOSL1 | TNFSF4 | SOX11 | CD15 |
| BLK | RASGRF1 | ERO1A | FPR1 | APH1B | SOX2 | CD11c |
| CD5 | RELB | FBP1 | FPR3 | DLL1 | FOXP3 | CD34 |
| CD96 | TGFB1 | G6PD | IER3 | DTX4 | NCR1 | CD83 |
| F2RL1 | TGFB2 | GLS | OLR1 | HES1 | CEACAM3 | PD-L1 |
| SLAMF7 | TGFB3 | GOT1 | PDZK1IP1 | JAG2 | FCGR3A/B | CD45 |
| DUSP1 | TGFBR2 | GOT2 | S100A12 | MAML2 | SIGLEC5 | CD124 |
| DUSP2 | A2M | LDHB | S100A8 | MFNG | CPA3 | CD66 |
| DUSP5 | COL11A1 | PC | S100A9 | NOTCH1 | HDC | CD3 |
| FAS | COL11A2 | PCK2 | SIGLEC8 | TCL1A | MS4A2 | CD13 |
| FGF13 | COL17A1 | PFKM | SIRPB2 | ACVR1C | TPSAB1/B2 | TPSAB1 |
| FGF9 | COL4A5 | PKM | SLC11A1 | BAMBI | CD68 | LILRA4 |
| KDR | COL5A1 | SGK1 | TLR1 | ID4 | MS4A4A | CD16 |
| KIT | COMP | SLC1A5 | TREM1 | INHBA | CD244 | PLTP |
| MAP3K5 | LAMA1 | SLC7A5 | TREM2 | SMAD5 | PTGER4 | ADM |
| NF1 | LAMB3 | AREG | NFKBIE | AXIN1 | HSD11B1 | CSTB |
| NFKB2 | LAMC2 | ARG1 | TNFRSF11A | DKK1 | FAM30A | PPARG |
| NGFR | LTBP1 | C5AR1 | TNFRSF11B | FZD8 | FCRL2 | RGCC |

Additional hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, where different epigenetic target regions are captured from first and second subsamples, the epigenetic target regions captured from the first subsample comprise hypermethylation target regions.

b. Hypomethylation Target Regions

In some embodiments, the epigenetic target region set comprises one or more hypomethylation target regions. In some embodiments, hypomethylation target regions are exclusively hypomethylated in one immune cell type or hypomethylated to a greater extent in one immune cell type than in any other immune cell type or in any other immune cell type within the same immune cell cluster. In some such embodiments, hypomethylation target regions indicate the levels of particular immune cell types from which the DNA originated, including rare immune cell types such as activated B cells (including memory B cells and plasma cells), activated T cells (including regulatory T cells (Tregs), CD4 effector memory T cells, CD4 central memory T cells, CD8 effector memory T cells, and CD8 central memory T cells), and natural killer (NK) cells. Methylation patterns of hypomethylation target regions that are useful for deconvoluting immune cell types may further change in certain disease states, such as cancer. Thus, in some embodiments, hypomethylation target regions that are useful for deconvoluting immune cell types are also useful for determining the likelihood that the subject from which the sample was obtained has cancer or precancer. In some such embodiments, hypomethylation target regions are useful for determining whether levels of particular immune cell types are abnormal and whether such abnormal levels are likely related to the presence of cancer or precancer, or if they are related to a different disease or condition other than cancer or precancer.

Additionally, global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation target regions in which a decrease in the level of observed methylation indicates an increased likelihood of the presence of cancer. Detection of such hypomethylation target regions, e.g., in conjunction with detection of hypomethylation target regions indicative of immune cell types, may further increase the specificity and/or sensitivity of methods described herein. In another example, as discussed above, hypomethylation target regions can include regions that do not necessarily differ in methylation in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation target regions. In some embodiments, hypomethylation target regions useful for determining the likelihood that a subject has cancer are different than the hypomethylation target regions useful for determining the levels of particular immune cell types. In some embodiments, at least some of the hypomethylation target regions useful for determining the likelihood that a subject has cancer are the same as the hypomethylation variable target regions useful for determining the levels of particular immune cell types.

In some embodiments, hypomethylation target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation target regions overlap or comprise one or both of these regions.

Additionally, hypomethylation target regions may be obtained, e.g., from Fox-Fisher et al., ElifeNov 29; 10 (2021), EpiDISH R package, Moss et al., Nat Commun 9:1 (2018), and Loyfer et al. bioRxiv https://doi.org/10.1101/2022.01.24.477547 (2022). In some embodiments, the hypomethylation target regions can be specific to one or more types of immune cells.

In some embodiments, where different epigenetic target regions are captured from first and second subsamples, the epigenetic target regions captured from the second subsample comprise hypomethylation target regions. In some embodiments, the epigenetic target regions captured from the second subsample comprise hypomethylation target regions and the epigenetic target regions captured from the first subsample comprise hypermethylation target regions.

c. CTCF Binding Regions

CTCF is a DNA-binding protein that contributes to chromatin organization and often colocalizes with cohesin. Perturbation of CTCF binding sites has been reported in a variety of different cancers. See, e.g., Katainen et al., Nature Genetics, doi:10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. Details regarding sequencing-based fragment length analysis are provided in Snyder et al., Cell 164:57-68 (2016); WO 2018/009723; and US20170211143A1, each of which are incorporated herein by reference.

Thus, perturbations of CTCF binding result in variation in the fragmentation patterns of cfDNA. As such, CTCF binding sites are a type of fragmentation variable target regions.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13.

Accordingly, in some embodiments, the epigenetic target region set includes CTCF binding regions. In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above.

In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the CTCF binding sites.

d. Transcription Start Sites

Transcription start sites may also show perturbations in neoplastic cells. For example, nucleosome organization at various transcription start sites in healthy cells of the hematopoietic lineage—which contributes substantially to cfDNA in healthy individuals—may differ from nucleosome organization at those transcription start sites in neoplastic cells. This results in different cfDNA patterns that can be detected by sequencing, as discussed generally in Snyder et al., Cell 164:57-68 (2016); WO 2018/009723; and US20170211143A1. In another example, transcription start sites may not necessarily differ epigenetically in cancerous tissue relative to DNA from healthy tissue of the same type, but do differ epigenetically (e.g., with respect to nucleosome organization) relative to cfDNA that is typical in healthy subjects. Where, for example, the presence of a cancer results in increased cell death, such as apoptosis, of cells of the tissue type corresponding to the cancer, such a cancer can be detected at least in part using such differences in transcription start sites.

Thus, perturbations of transcription start sites also result in variation in the fragmentation patterns of cfDNA. As such, transcription start sites are also a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34 (Database issue): D86-D89 (2006), which is incorporated herein by reference.

Accordingly, in some embodiments, the epigenetic target region set includes transcriptional start sites. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the transcription start sites.

e. Focal Amplifications

Although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

### F. Methylation Control Regions or Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes negative control regions that are expected to be hypomethylated or unmethylated in essentially all samples. In some embodiments, the epigenetic target region set includes positive control regions that are expected to be hypermethylated in essentially all samples.

### 2. Sequence-Variable Target Region Set

In some embodiments, the sequence-variable target region set comprises a plurality of regions known to undergo somatic mutations (e.g., single nucleotide variations and/or indels) in cancer. The single nucleotide variations and/or indels may be relative to a reference sequence, e.g., a published human genome sequence, such as the GRCh38 human genome assembly.

In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample. Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 4 and Table 5. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3. In some embodiments, a sequence-variable target region set used in the methods of the present disclosure comprises at least 5, at least 10, at least 15, at least 20, at least 25, at least 30, at least 35, at least 40, at least 45, at least 50, at least 55, at least 60, at least 65, or 70 of the SNVs of Table 4. In some embodiments, a sequence-variable target region set used in the methods of the present disclosure comprises at least 1, at least 2, at least 3, at least 4, at least 5, or 6 of the fusions of Table 4. In some embodiments, a sequence-variable target region set used in the methods of the present disclosure comprise at least a portion of at least 1, at least 2, or 3 of the indels of Table 4. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 5. In some embodiments, a sequence-variable target region set used in the methods of the present disclosure comprises at least 5, at least 10, at least 15, at least 20, at least 25, at least 30, at least 35, at least 40, at least 45, at least 50, at least 55, at least 60, at least 65, at least 70, or 73 of the SNVs of Table 5. In some embodiments, a sequence-variable target region set used in the methods of the present disclosure comprises at least 1, at least 2, at least 3, at least 4, at least 5, or 6 of the fusions of Table 5. In some embodiments, a sequence-variable target region set used in the methods of the present disclosure comprises at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 5. Each of these genomic locations of interest may be identified as a backbone region or hot-spot region for a given panel. An example of a listing of hot-spot genomic locations of interest may be found in Table 6. In some embodiments, a sequence-variable target region set used in the methods of the present disclosure comprises at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 6. Each hot-spot genomic region is listed with several characteristics, including the associated gene, chromosome on which it resides, the start and stop position of the genome representing the gene's locus, the length of the gene's locus in base pairs, the exons covered by the gene, and the critical feature (e.g., type of mutation) that a given genomic region of interest may seek to capture.

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 5

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |

NTRK3

TABLE 6

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5:1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

Additionally or alternatively, suitable target region sets are available from the literature. For example, Gale et al., PLOS One 13: e0194630 (2018), which is incorporated herein by reference, describes a panel of 35 cancer-related gene targets that can be used as part or all of a sequence-variable target region set. These 35 targets are AKTI, ALK, BRAF, CCND1, CDK2A, CTNNB1, EGFR, ERBB2, ESR1, FGFR1, FGFR2, FGFR3, FOXL2, GAT A3, GNA11, GNAQ, GNAS, HRAS, IDH1, IDH2, KIT, KRAS, MED 12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed above.

### H. Subjects

In some embodiments, the DNA (e.g., cfDNA) is obtained from a subject having a cancer or a precancer, an infection, transplant rejection, or other disease directly or indirectly affecting the immune system. In some embodiments, the DNA (e.g., cfDNA) is obtained from a subject suspected of having a cancer or a precancer, an infection, transplant rejection, or other disease directly or indirectly affecting the immune system. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

I. Pooling of DNA from Samples or Subsamples or Portions Thereof

In some embodiments, the methods herein comprise preparing one or more pools comprising tagged DNA from a plurality of partitioned subsamples. In some embodiments, a pool comprises at least a portion of the DNA of a hypomethylated partition and at least a portion of the DNA of a hypermethylated partition. Target regions, e.g., including epigenetic target regions and/or sequence-variable target regions, may be captured from a pool. The steps of capturing a target region set from at least an aliquot or portion of a sample or subsample described elsewhere herein encompass capture steps performed on a pool comprising DNA from first and second subsamples. A step of amplifying DNA in a pool may be performed before capturing target regions from the pool. The capturing step may have any of the features described for capturing steps elsewhere herein.

In some embodiments, the methods comprise preparing a first pool comprising at least a portion of the DNA of a hypomethylated partition. In some embodiments, the methods comprise preparing a second pool comprising at least a portion of the DNA of a hypermethylated partition. In some embodiments, the methods comprise capturing at least a first set of target regions from the first pool, wherein the first set comprises sequence-variable target regions. A step of amplifying DNA in the first pool may be performed before this capture step. In some embodiments, capturing the first set of target regions from the first pool comprises contacting the DNA of the first pool with a first set of target-specific probes, wherein the first set of target-specific probes comprises target-binding probes specific for the sequence-variable target regions. In some embodiments, the methods comprise capturing a second plurality of sets of target regions from the second pool, wherein the second plurality comprises sequence-variable target regions and epigenetic target regions. A step of amplifying DNA in the second pool may be performed before this capture step. In some embodiments, capturing the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions.

In some embodiments, sequence-variable target regions are captured from a second portion of a partitioned subsample. The second portion may include some, a majority, substantially all, or all of the DNA of the subsample that was not included in the pool. The regions captured from the pool and from the subsample may be combined and analyzed in parallel.

The epigenetic target regions may show differences in methylation levels and/or fragmentation patterns depending on whether they originated from a particular cell or tissue type or from a tumor or from healthy cells, as discussed elsewhere herein. The sequence-variable target regions may show differences in sequence depending on whether they originated from a tumor or from healthy cells. Analysis of epigenetic target regions from a hypomethylated partition may be less informative in some applications than analysis of sequence-variable target regions from hypermethylated and hypomethylated partitions and epigenetic target regions from a hypermethylated partition. As such, in methods where sequence-variable target regions and epigenetic target regions are being captured, the latter may be captured to a lesser extent than one or more of the sequence-variable target regions are captured from the hypermethylated and hypomethylated partitions and/or to a lesser extent that epigenetic target regions are captured from a hypermethylated partition. For example, sequence-variable target regions can be captured from a portion of a hypomethylated partition that is not pooled with a hypermethylated partition, and the pool can be prepared with some (e.g., a majority, substantially all, or all) of the DNA from a hypermethylated partition and none or some (e.g., a minority) of the DNA from a hypomethylated partition. Such approaches can reduce or eliminate sequencing of epigenetic target regions from hypomethylated partitions, thereby reducing the amount of sequencing data that suffices for further analysis.

In some embodiments, including a minority of the DNA of a hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of a hypomethylated partition, e.g., less than about 50% of the DNA of a hypomethylated partition, such as less than or equal to about 45%, 40%, 35%, 30%, 25%, 20%, 15%, 10%, or 5% of the DNA of a hypomethylated partition. In some embodiments, the pool comprises about 5%-25% of the DNA of a hypomethylated partition. In some embodiments, the pool comprises about 10%-20% of the DNA of a hypomethylated partition. In some embodiments, the pool comprises about 10% of the DNA of a hypomethylated partition. In some embodiments, the pool comprises about 15% of the DNA of a hypomethylated partition. In some embodiments, the pool comprises about 20% of the DNA of a hypomethylated partition.

In some embodiments, the pool comprises a portion of a hypermethylated partition, which may be at least about 50% of the DNA of a hypermethylated partition. For example, the pool may comprise at least about 55%, 60%, 65%, 70%, 75%, 80%, 85%, 90%, or 95% of the DNA of a hypermethylated partition. In some embodiments, the pool comprises 50-55%, 55-60%, 60-65%, 65-70%, 70-75%, 75-80%, 80-85%, 85-90%, 90-95%, or 95-100% of the DNA of a hypermethylated partition. In some embodiments, the second pool comprises all or substantially all of the DNA of a hypermethylated partition.

In some embodiments, a first pool comprises substantially all or all of the DNA of a hypomethylated partition (e.g., wherein a second pool does not comprise DNA of a hypomethylated partition. In some embodiments, the second pool does not comprise DNA of a hypomethylated partition (e.g., wherein the first pool comprises substantially all or all of the DNA of a hypomethylated partition).

In some embodiments, a second pool comprises a portion of a hypermethylated partition, which may be any of the values and ranges set forth above with respect to a hypomethylated partition. In some embodiments, the second pool comprises all or substantially all of the DNA of a hypermethylated partition.

In an exemplary embodiment, after partitioning, the partitions separately undergo end repair and ligation to adapters comprising molecular barcodes and are then amplified separately. After the amplification, amplified molecules are enriched (still keeping the partitions separate). Post-enrichment, the enriched DNA are pooled according to any of the embodiments described herein, and then amplified again. After amplification, the molecules are sequenced.

In various embodiments, the methods further comprise sequencing the captured DNA, e.g., to different degrees of sequencing depth for the epigenetic and sequence-variable target region sets, consistent with the discussion above.

### J. Sequencing

In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms.

In some embodiments, sequencing comprises detecting and/or distinguishing unmodified and modified nucleobases. For example, PacBio sequencing (e.g., single-molecule real-time (SMRT) sequencing) offers the ability to directly detect of, e.g., 5-methylcytosine and 5-hydroxymethylcytosine as well as unmodified cytosine. See, e.g., Schatz., Nature Methods. 14 (4): 347-348 (2017); and U.S. Pat. No. 9,150,918. Also, Oxford nanopore sequencing systems (e.g., MinION sequencer) that can directly detect methylation of DNA (for example: 5-methylcytosine and 5-hydroxymethylcytosine) can be used here. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously. Similarly, Ion Torrent sequencing may also be used to directly detect methylation. Thus, in some embodiments, methylation status can be determined during sequencing, e.g., without or independently of a partitioning step or a conversion procedure such as bisulfite treatment.

The sequencing reactions can be performed on one or more forms of nucleic acids, such as those known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can performed on at least 5, 10, 20, 70, 100, 200 or 500 different genes, or at most 5000, 2500, 1000, 500 or 100 different genes. Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base). 1.

### 1. Differential Depth of Sequencing

In some embodiments, nucleic acids corresponding to a sequence-variable target region set are sequenced to a greater depth of sequencing than nucleic acids corresponding to an epigenetic target region set. For example, the depth of sequencing for nucleic acids corresponding to sequence variant target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to an epigenetic target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater.

In some embodiments, DNA corresponding to a sequence-variable target region set, and/or to an epigenetic target region set are sequenced concurrently, e.g., in the same sequencing cell (such as the flow cell of an Illumina sequencer) and/or in the same composition, which may be a combined or pooled composition resulting from recombining separately captured sets or a composition obtained by, e.g., capturing the cfDNA corresponding to the sequence-variable target region set, and/or the captured cfDNA corresponding to an epigenetic target region set in the same vessel.

### K. Analysis

In some embodiments, any of the methods disclosed herein comprises determining a likelihood that the subject from which the DNA was obtained has a disease or disorder related to the immune system, such as an infection, transplant rejection, or cancer or precancer. an indication of cancer

In some embodiments, any of the methods disclosed herein comprises identifying the presence of DNA produced by a tumor (or neoplastic cells, or cancer cells) or by precancer cells. In some embodiments, a method described herein comprises determining an indication of cancer in the subject. In some such embodiments, determination of the indication of cancer facilitates detection or diagnosis or cancer or precancer, or determination of cancer prognosis or cancer treatment options. For example, determining the metrics from the one or more classification regions and the one or more control regions can help in determining the indication of cancer. In some embodiments, the metrics can be used to determine the tumor fraction of a sample.

The present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. For example, the change in the tumor fraction or determining the methylation status of one or regions can be useful in determining whether the patient is responding to the treatment or not. In another example, perhaps certain treatment options may be correlated with methylation profiles of cancers over time. This correlation may be useful in selecting a therapy.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer or precancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, epigenetic variation, or other mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

An exemplary method for determining an indication of cancer through NGS comprises the following steps:
1. Extracting cfDNA from a blood sample
2. Partitioning cfDNA into a plurality of partitions by contacting the DNA with an agent that recognizes a modified cytosine, such as methyl cytosine, in the DNA
3. Ligating the partitions with adapters comprising molecular barcodes
4. Treating the hyper and/or intermediate partitions with one or more MSREs and/or treating the hypo partition with one or more MDREs
5. Amplifying the partitions post digestion via PCR amplification
6. Capturing DNA comprising hypermethylated target regions, hypomethylated target regions and control regions using target-specific probes.
7. Amplifying the captured DNA and assaying in multiplex on an NGS instrument.
8. Analyzing NGS data using one or more methods disclosed herein to determine the indication of cancer.

Another exemplary method for determining an indication of cancer through NGS comprises the following steps:
1. Extracting cfDNA from a blood sample.
3. Ligating the cfDNA with adapters comprising molecular barcodes
4. Subjecting the ligated cfDNA to a methylation-sensitive conversion method
6. Capturing DNA comprising hypermethylated target regions, hypomethylated target regions and control regions using target-specific probes, wherein the probes are designed such that they can be targeted to capture converted or unconverted molecules depending on the type of methylation-sensitive conversion method and the target regions (whether hypermethylated target regions or hypomethylated target regions) being captured.
7. Amplifying the captured DNA and assaying in multiplex on an NGS instrument.
8. Analyzing NGS data using one or more methods disclosed herein to determine the indication of cancer.

Another exemplary method for determining methylation status of a target region (e.g., promoter region) through NGS comprises the following steps:
1. Extracting cfDNA from a blood sample
2. Partitioning cfDNA into a plurality of partitions by contacting the DNA with an agent that recognizes a modified cytosine, such as methyl cytosine, in the DNA
3. Ligating the partitions with adapters comprising molecular barcodes
4. Treating the hyper and/or intermediate partitions with one or more MSREs and/or treating the hypo partition with one or more MDREs
5. Amplifying the partitions post digestion via PCR amplification
6. Capturing DNA comprising hypermethylated target regions, hypomethylated target regions and control regions using target-specific probes.
7. Amplifying the captured DNA and assaying in multiplex on an NGS instrument.
8. Analyzing NGS data using one or more methods disclosed herein to determine whether the target region (e.g., promoter region) is methylated or not.

In some embodiments of methods described herein, molecular barcodes consist of nucleotides that are not altered by a procedure that affects a first nucleobase in the DNA differently from a second nucleobase in the DNA, such as any of those described herein (e.g., mC along with A, T, and G where the procedure is bisulfite conversion or any other conversion that does not affect mC; hmC along with A, T, and G where the procedure is a conversion that does not affect hmC; etc.). In some embodiments of methods described herein, the molecular tags do not comprise nucleotides that are altered by a procedure that affects a first nucleobase in the DNA differently from a second nucleobase in the DNA, such as any of those described herein (e.g., the tags do not comprise unmodified C where the procedure is bisulfite conversion or any other conversion that affects C; the tags do not comprise mC where the procedure is a conversion that affects mC; the tags do not comprise hmC where the procedure is a conversion that affects hmC; etc.).

### Additional Features of Certain Disclosed Methods

### A. Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

In some embodiments, the sample comprises plasma. The volume of plasma obtained can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000 (10⁴) haploid human genome equivalents and, in the case of cfDNA, about 200 billion (2×IOⁿ) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., precancer cells or cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of cell-free nucleic acids in a sample before amplification range from about 1 fg to about 1 pg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic

acid molecules. The method can comprise obtaining 1 femtogram (fg) to 200 ng-Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation step in which cell-free nucleic acids, as found in solution, are separated from intact cells and other non soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as C 1 DNA, DNA or protein for bisulfite sequencing, hybridization, and/or ligation, may be added throughout the reaction to optimize certain aspects of the procedure such as yield. After such processing, samples can include various forms of nucleic acid including double stranded DNA, single stranded DNA, and single stranded RNA. In some embodiments, single stranded DNA and RNA can be converted to double stranded forms so they are included in subsequent processing and analysis steps. DNA molecules can be linked to adapters at either one end or both ends. Typically, double-stranded molecules are blunt ended by treatment with a polymerase with a 5′-3′ polymerase and a 3′-5′ exonuclease (or proof-reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### B. Tags

Tags comprising barcodes can be incorporated into or otherwise joined to adapters. Tags can be incorporated by ligation, overlap extension PCR among other methods.

### i) Molecular Tagging Strategies

Molecular tagging refers to a tagging practice that allows one to differentiate among DNA molecules from which sequence reads originated. Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*Z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required. Generally, assignment of unique or non-unique tags barcodes in reactions follows methods and systems described by U.S. patent applications 20010053519, 20030152490, 20110160078, and U.S. Pat. Nos. 6,582,908 and 7,537,898 and 9,598,731. Tags can be linked to sample nucleic acids randomly or non-randomly. The unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

A preferred format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

### C. Amplification

Sample nucleic acids flanked by adapters can be amplified by PCR and other amplification methods. Amplification is typically primed by primers that anneal or bind to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters, which result in amplification of at least 50, 60, 70 or 80% of double stranded nucleic acids before linking to adapters. Preferably the present methods increase the amount or number of amplified molecules relative to control methods performed with T-tailed adapters alone by at least 10, 15 or 20%.

### D. Capture Moieties

As discussed above, nucleic acids in a sample can be subject to a capture step, in which molecules having target regions are captured for subsequent analysis. Target capture can involve use of probes (e.g., oligonucleotides) labeled with a capture moiety, such as biotin, and a second moiety or binding partner that binds to the capture moiety, such as streptavidin. In some embodiments, a capture moiety and binding partner can have higher and lower capture yields for different sets of target regions, such as those of the sequence-variable target region set and the epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

### E. Collections of Target-Specific Probes

In some embodiments, a collection of target-specific probes is used in a method comprising an epigenetic target region set and/or a sequence-variable target region set, as described herein. In some embodiments, the collection of target-specific probes comprises target binding probes specific for a sequence-variable target region set and target-binding probes specific for an epigenetic target region set. In some embodiments, the capture yield of the target binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of target-specific probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of target-specific probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the target-specific probes specific for the sequence-variable target region set are present at a higher concentration than the target-specific probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the target-specific probes specific for the sequence-variable target region set have a higher affinity for their targets than the target-specific probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the T sugar position, and LNA nucleotides, can increase stability of double-stranded nucleic acids, indicating that oligonucleotides with such modifications have relatively higher affinity for their complementary sequences. See, e.g., Severin et ah, Nucleic Acids Res. 39:8740-8751 (2011); Freier et ah, Nucleic Acids Res. 25:4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the target-specific probes specific for the sequence-variable target region set have modifications that increase their affinity for their targets. In some embodiments, alternatively or additionally, the target-specific probes specific for the epigenetic target region set have modifications that decrease their affinity for their targets. In some embodiments, the target-specific probes specific for the sequence-variable target region set have longer average lengths and/or higher average melting temperatures than the target-specific probes specific for the epigenetic target region set. These embodiments may be combined with each other and/or with differences in concentration as discussed above to achieve a desired fold difference in capture yield, such as any fold difference or range thereof described above.

In some embodiments, the target-specific probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the target-specific probes specific for the sequence-variable target region set and/or the target-specific probes specific for the epigenetic target region set comprise a capture moiety as discussed above, e.g., probes comprising capture moieties and sequences selected to tile across a panel of regions, such as genes.

In some embodiments, the target-specific probes are provided in a single composition.

The single composition may be a solution (liquid or frozen). Alternatively, it may be a lyophilizate.

Alternatively, the target-specific probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### i) Probes Specific for Epigenetic Target Regions

The probes for the epigenetic target region set may comprise probes specific for one or more types of target regions likely to differentiate DNA originating from different types of immune cells, including rare immune cell types, and/or to differentiate DNA from precancerous or neoplastic (e.g., tumor or cancer) cells from healthy cells, e.g., non-neoplastic circulating cells. Exemplary types of such regions are discussed in detail herein. The probes for the epigenetic target region set may also comprise probes for one or more control regions, e.g., as described herein.

In some embodiments, the probes for the epigenetic target region probe set have a footprint of at least 100 kb, e.g., at least 200 kb, at least 300 kb, or at least 400 kb. In some embodiments, the probes for the epigenetic target region set have a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb. In some embodiments, the probes for the epigenetic target region probe set have a footprint of at least 5 kb, e.g., at least 10, 20, or 50 kb. a. Hypermethylation target regions.

In some embodiments, for the methods using methylation-sensitive conversion (e.g., bisulfite or EM-seq), the probes can be designed to target either the converted molecules or unconverted molecules depending on the type of methylation-sensitive conversion and the target region being enriched. For example, if bisulfite treatment is used, the unmethylated cytosines in the DNA molecules will be converted to dihydrouracil and methylated cytosines will remain unconverted as cytosine. For capturing DNA molecules in the hypermethylated target regions (where the molecules of interest to cancer or any other disease under investigation will be hypermethylated), the probes can be designed to capture the unconverted molecules, whereas for capturing molecules in the hypomethylated target regions (where the molecules of interest to cancer or any other disease under investigation will be hypomethylated or unmethylated), the probes can be designed to capture the converted molecules

In some embodiments, the probes for the epigenetic target region set comprise probes specific for one or more hypermethylation target regions. The hypermethylation target regions may be any of those set forth above. For example, in some embodiments, the probes specific for hypermethylation target regions comprise probes specific for a plurality of loci that are differentially methylated in different immune cell types. In some embodiments, each immune cell type specific hypermethylation target region comprises at least one CpG site that is methylated with a frequency greater than or equal to 0.3, 0.4, 0.5, or 0.6 in one immune cell type and with a frequency less than or equal to 0.1, 0.2, or 0.3 in all other immune cell types. In some embodiments, each immune cell type specific hypermethylation target region comprises at least two CpG sites within 100 base pairs of each other that are each methylated with a frequency greater than or equal to 0.3, 0.4, 0.5, or 0.6 in one immune cell type and with a frequency less than or equal to 0.1, 0.2, or 0.3 in all other immune cell types. In some such embodiments, each immune cell type specific hypermethylation target region comprises a total of at least 2, 3, 4, 5, 6, 7, 8, 9, or 10 CpG sites within 150 base pairs or within 200 base pairs, wherein fewer than three of the at least 2, 3, 4, 5, 6, 7, 8, 9, or 10 CpG sites are methylated with a frequency greater than 0.1, 0.2, or 0.3 in any normal tissue type. In some embodiments, each immune cell type specific epigenetic target region set comprises at least 3, at least 5, at least 10, at least 20, or at least 30 hypermethylation target regions that are uniquely hypermethylated in each one of the immune cell types that are identified in the method.

In some embodiments, the probes specific for hypermethylation target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2.

In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers. b. Hypomethylation target regions.

In some embodiments, the probes for the epigenetic target region set comprise probes specific for one or more hypomethylation target regions. The hypomethylation target regions may be any of those set forth above. For example, in some embodiments, the probes specific for hypomethylation target regions comprise probes specific for a plurality of loci that are differentially methylated in different immune cell types. In some embodiments, each immune cell type specific hypomethylation target region comprises at least one CpG site that is methylated with a frequency less than or equal to 0.1, 0.2, or 0.3 in one immune cell type and with a frequency greater than or equal to 0.3, 0.4, 0.5, or 0.6 in all other immune cell types. In some embodiments, each immune cell type specific hypomethylation target region comprises at least two CpG sites within 100 base pairs of each other that are each methylated with a frequency less than or equal to 0.1, 0.2, or 0.3 in one immune cell type and with a frequency greater than or equal to 0.3, 0.4, 0.5, or 0.6 in all other immune cell types. In some such embodiments, each immune cell type specific hypomethylation target region comprises a total of at least 2, 3, 4, 5, 6, 7, 8, 9, or 10 CpG sites within 150 base pairs or within 200 base pairs, wherein fewer than three of the at least 2, 3, 4, 5, 6, 7, 8, 9, or 10 CpG sites are methylated with a frequency less than 0.1, 0.2, or 0.3 in any normal tissue type. In some embodiments, each immune cell type specific epigenetic target region set comprises at least 3, at least 5, at least 10, at least 20, or at least 30 hypomethylation target regions that are uniquely hypomethylated in each one of the immune cell types that are identified in the method.

In some embodiments, the probes specific for one or more hypomethylation target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation target regions include probes specific for regions overlapping or comprising nucleotides 8403565-8953708 and/or 151104701-151106035 of human chromosome 1.

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites. d. Transcription start sites.

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAFI. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets

### Control Regions

In some embodiments, the probes specific for the epigenetic target region set include probes specific for positive control regions that are expected to be methylated in essentially all samples. In some embodiments, the probes specific for the epigenetic target region set include probes specific for negative control regions that are expected to be hypomethylated or unmethylated in essentially all samples.

### 2) Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets. In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 4. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 4. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 5. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 5. In some embodiments, probes specific for the sequence-variable target region set comprise probes specific for at least 1, at least 2, at least 3, at least 4, at least 5, or 6 of the fusions of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 6.

In some embodiments, the probes specific for the sequence-variable target region set comprise probes specific for target regions from at least 10, 20, 30, or 35 cancer-related genes, such as AKTI, ALK, BRAF, CCND1, CDK2A, CTNNB1, EGFR, ERBB2, ESR1, FGFR1, FGFR2, FGFR3, FOXL2, GAT A3, GNA11, GNAQ, GNAS, HRAS, IDH1, IDH2, KIT, KRAS, MED 12, MET, MYC, NFE2L2, NRAS, PDGFRA, PIK3CA, PPP2R1A, PTEN, RET, STK11, TP53, and U2AF 1.

### Precision Treatments

The precision diagnostics provided by the improved computer system 110 may result in precision treatment plans, which may be identified by the computer system 110 (and/or curated by health professionals). For example, one type of precision diagnostic and treatment may relate to genes in the homologous recombination repair (HRR) pathway.

Homologous recombination is a type of genetic recombination in which nucleotide sequences are exchanged between two similar or identical molecules of DNA. It is most widely used by cells to accurately repair harmful breaks that occur on both strands of DNA, known as double-strand breaks (DSB). HRR provides a mechanism for the error-free removal of damage present in DNA that has replicated (S and G2 phases), to eliminate chromosomal breaks before the cell division occurs. The primary model for how homologous recombination repairs double-strand breaks in DNA is homologous recombination repair pathway which mediates the double-strand break repair (DSBR) pathway and the synthesis-dependent strand annealing (SDSA) pathway. Germline and somatic deficiencies in homologous recombination genes have been strongly linked to breast, ovarian and prostate cancers.

The number and types of variant nucleotides in a sample can provide an indication of the amenability of the subject providing the sample to treatment, i.e., therapeutic intervention. For example, various poly ADP ribose polymerase (PARP) inhibitors have been shown to stop the growth of tumors from breast, ovarian and prostate cancers caused by hereditary mutations in the BRCA1 or BRCA2 genes. Some of these therapeutic agents may inhibit base excision repair (BER), which may compensate for the deficiency of HRR.

On the other hand, certain BRCA and HRR wildtype patients may not achieve clinical benefit from treatment with a PARP inhibitor. Furthermore, not all ovarian cancer patients with a BRCA mutation will respond to a PARP inhibitor. Moreover, different types of mutations may indicate different therapies. For example, somatic heterozygous deletions in HRR genes may indicate a different therapy than somatic homozygous deletions. Thus, the state of genetic material may influence therapy. In one example, a PARP inhibitor may be administered to an individual harboring a somatic homozygous deletion in a HRR gene, but not to an individual harboring a wildtype allele or somatic heterozygous deletions in the HRR gene.

In some implementations, a subject having HRD as determined by any of the methods disclosed may be administered a targeted therapy. The targeted therapy may comprise a PARP inhibitor. Examples of PARP inhibitors that may be administered include one or more of: VELIPARIB, OLAPARIB, TALAZOPARIB, RUCAPARIB, NIRAPARIB, PAMIPARIB, CEP 9722 (Cephalon), E7016 (Eisai), E7449 (Eisai, a PARP ½ and tankyrase ½ inhibitor), or 3-Aminobenzamide. In some implementations, the targeted therapy may comprise at least one base excision repair (BER) inhibitor. For example, OLAPARIB may inhibit BER. In certain implementations, the targeted therapy may comprise combination of a PARP inhibitor and radiotherapy. In an implementation, the combination of a PARP inhibitor and radiotherapy would permit the PARP inhibitor to lead to formation of double strand breaks from the single-strand breaks generated by the radiotherapy in tumor tissue (e.g., tissue with BRCA1/BRCA2 mutations). This combination can provide more powerful therapy per radiation dose.

### Customized Therapies and Related Administrations

In some implementations, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. In certain implementations, the therapy administered to a subject may comprise at least one chemotherapy drug. In some implementations, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some implementations, the chemotherapy administered to a subject may comprise FOLFOX or FOLFIRI. Typically, therapies include at least one immunotherapy (or an immunotherapeutic agent). Immunotherapy refers generally to methods of enhancing an immune response against a given cancer type. In certain implementations, immunotherapy refers to methods of enhancing a T cell response against a tumor or cancer.

In some implementations, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain implementations, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain implementations, the inhibitory immune checkpoint molecule is CTLA4 or PD-1. In other implementations, the inhibitory immune checkpoint molecule is a ligand for PD-1, such as PD-L1 or PD-L2. In other implementations, the inhibitory immune checkpoint molecule is a ligand for CTLA4, such as CD80 or CD86. In other implementations, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain implementations, the immunotherapy or immunotherapeutic agent is an antagonist of an inhibitory immune checkpoint molecule. In certain implementations, the inhibitory immune checkpoint molecule is PD-1. In certain implementations, the inhibitory immune checkpoint molecule is PD-L1. In certain implementations, the antagonist of the inhibitory immune checkpoint molecule is an antibody (e.g., a monoclonal antibody). In certain implementations, the antibody or monoclonal antibody is an anti-CTLA4, anti-PD-1, anti-PD-L1, or anti-PD-L2 antibody. In certain implementations, the antibody is a monoclonal anti-PD-1 antibody. In some implementations, the antibody is a monoclonal anti-PD-L1 antibody. In certain implementations, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain implementations, the anti-PD-1 antibody is one or more of pembrolizumab (Keytruda®) or nivolumab (Opdivo®). In certain implementations, the anti-CTLA4 antibody is ipilimumab (Yervoy®). In certain implementations, the anti-PD-L1 antibody is one or more of atezolizumab (Tecentriq®), avelumab (Bavencio®), or durvalumab (Imfinzi®).

In certain implementations, the immunotherapy or immunotherapeutic agent is an antagonist (e.g. antibody) against CD80, CD86, LAG3, KIR, TIM3, GAL9, or A2aR. In other implementations, the antagonist is a soluble version of the inhibitory immune checkpoint molecule, such as a soluble fusion protein comprising the extracellular domain of the inhibitory immune checkpoint molecule and an Fc domain of an antibody. In certain implementations, the soluble fusion protein comprises the extracellular domain of CTLA4, PD-1, PD-L1, or PD-L2. In some implementations, the soluble fusion protein comprises the extracellular domain of CD80, CD86, LAG3, KIR, TIM3, GAL9, or A2aR. In one implementation, the soluble fusion protein comprises the extracellular domain of PD-L2 or LAG3.

In certain implementations, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain implementations, the immune checkpoint molecule is a co-stimulatory molecule selected from CD28, inducible T cell co-stimulator (ICOS), CD137, OX40, or CD27. In other implementations, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain implementations, the immunotherapy or immunotherapeutic agent is an agonist of a co-stimulatory checkpoint molecule. In certain implementations, the agonist of the co-stimulatory checkpoint molecule is an agonist antibody and preferably is a monoclonal antibody. In certain implementations, the agonist antibody or monoclonal antibody is an anti-CD28 antibody. In other implementations, the agonist antibody or monoclonal antibody is an anti-ICOS, anti-CD137, anti-OX40, or anti-CD27 antibody. In other implementations, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain implementations, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

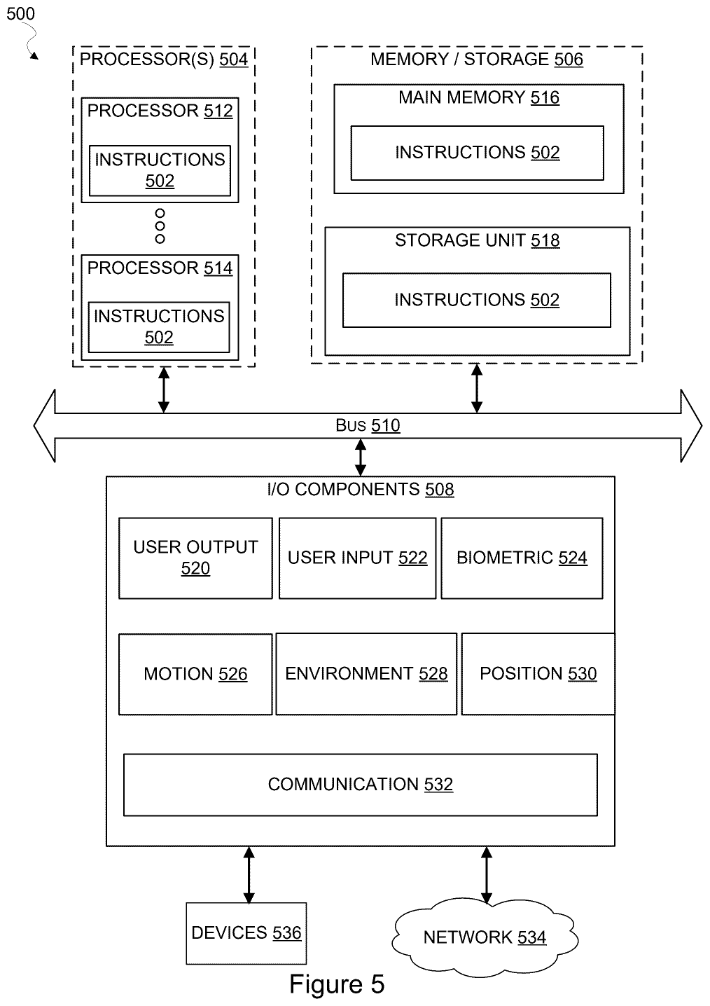

FIG. 5 is a block diagram illustrating components of a machine 500, according to some example implementations, able to read instructions from a machine-readable medium (e.g., a machine-readable storage medium) and perform any one or more of the methodologies discussed herein. Specifically, FIG. 5 shows a diagrammatic representation of the machine 500 in the example form of a computer system, within which instructions 502 (e.g., software, a program, an application, an applet, an app, or other executable code) for causing the machine 500 to perform any one or more of the methodologies discussed herein may be executed. As such, the instructions 502 may be used to implement modules or components described herein. The instructions 502 transform the general, non-programmed machine 500 into a particular machine 500 programmed to carry out the described and illustrated functions in the manner described. In alternative implementations, the machine 500 operates as a standalone device or may be coupled (e.g., networked) to other machines. In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 502, sequentially or otherwise, that specify actions to be taken by machine 500. Further, while only a single machine 500 is illustrated, the term “machine” shall also be taken to include a collection of machines that individually or jointly execute the instructions 502 to perform any one or more of the methodologies discussed herein.

The machine 500 may include processors 504, memory/storage 506, and I/O components 508, which may be configured to communicate with each other such as via a bus 510. In an example implementation, the processors 504 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 512 and a processor 514 that may execute the instructions 502. The term “processor” is intended to include multi-core processors 504 that may comprise two or more independent processors (sometimes referred to as “cores”) that may execute instructions 502 contemporaneously. Although FIG. 5 shows multiple processors 504, the machine 500 may include a single processor 512 with a single core, a single processor 512 with multiple cores (e.g., a multi-core processor), multiple processors 512, 514 with a single core, multiple processors 512, 514 with multiple cores, or any combination thereof.

The memory/storage 506 may include memory, such as a main memory 516, or other memory storage, and a storage unit 518, both accessible to the processors 504 such as via the bus 510. The storage unit 518 and main memory 516 store the instructions 502 embodying any one or more of the methodologies or functions described herein. The instructions 502 may also reside, completely or partially, within the main memory 516, within the storage unit 518, within at least one of the processors 504 (e.g., within the processor's cache memory), or any suitable combination thereof, during execution thereof by the machine 500. Accordingly, the main memory 516, the storage unit 518, and the memory of processors 504 are examples of machine-readable media.

The I/O components 508 components 508 may include a wide variety of components to receive input, provide output, produce output, transmit information, exchange information, capture measurements, and so on. The specific I/O components 508 that are included in a particular machine 500 will depend on the type of machine. For example, portable machines such as mobile phones will likely include a touch input device or other such input mechanisms, while a headless server machine will likely not include such a touch input device. It will be appreciated that the I/O components 508 components 508 may include many other components that are not shown in FIG. 5. The I/O components 508 are grouped according to functionality merely for simplifying the following discussion and the grouping is in no way limiting. In various example implementations, the I/O components 508 components 508 may include user output components 520 and user input components 522. The user output components 520 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 522 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like.

In further example implementations, the I/O components 508 components 508 may include biometric components 524, motion components 526, environmental components 528, or position components 530 among a wide array of other components. For example, the biometric components 524 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 526 may include acceleration sensor components (e.g., accelerometer), gravitation sensor components, rotation sensor components (e.g., gyroscope), and so forth. The environmental components 528 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 530 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 508 may include communication components 532 operable to couple the machine 500 to a network 534 or devices 536. For example, the communication components 532 may include a network interface component or other suitable device to interface with the network 534. In further examples, communication components 532 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 536 may be another machine 500 or any of a wide variety of peripheral devices (e.g., a peripheral device coupled via a USB).

Moreover, the communication components 532 may detect identifiers or include components operable to detect identifiers. For example, the communication components 532 may include radio frequency identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 532, such as location via Internet Protocol (IP) geo-location, location via Wi-Fi® signal triangulation, location via detecting an NFC beacon signal that may indicate a particular location, and so forth.

As used herein, “component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an ASIC. A hardware component may also include programmable logic or circuitry that is temporarily configured by software to perform certain operations. For example, a hardware component may include software executed by a general-purpose processor 504 or other programmable processor. Once configured by such software, hardware components become specific machines (or specific components of a machine 500) uniquely tailored to perform the configured functions and are no longer general-purpose processors 504. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering implementations in which hardware components are temporarily configured (e.g., programmed), each of the hardware components need not be configured or instantiated at any one instance in time. For example, where a hardware component comprises a general-purpose processor 504 configured by software to become a special-purpose processor, the general-purpose processor 504 may be configured as respectively different special-purpose processors (e.g., comprising different hardware components) at different times. Software accordingly configures a particular processor 512 processor 512, 514 or processors 504, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In implementations in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices, and can operate on a resource (e.g., a collection of information). The various operations of example methods described herein may be performed, at least partially, by one or more processors 504 that are temporarily configured (e.g., by software) or permanently configured to perform the relevant operations. Whether temporarily or permanently configured, such processors 504 may constitute processor-implemented components that operate to perform one or more operations or functions described herein. As used herein, “processor-implemented component” refers to a hardware component implemented using one or more processors 504. Similarly, the methods described herein may be at least partially processor-implemented, with a particular processor 512 processor 512, 514 or processors 504 being an example of hardware. For example, at least some of the operations of a method may be performed by one or more processors 504 or processor-implemented components. Moreover, the one or more processors 504 may also operate to support performance of the relevant operations in a “cloud computing” environment or as a “software as a service” (SaaS). For example, at least some of the operations may be performed by a group of computers (as examples of machines 1000 including processors 504), with these operations being accessible via a network 534 (e.g., the Internet) and via one or more appropriate interfaces (e.g., an API). The performance of certain of the operations may be distributed among the processors, not only residing within a single machine 500, but deployed across a number of machines. In some example implementations, the processors 504 or processor-implemented components may be located in a single geographic location (e.g., within a home environment, an office environment, or a server farm). In other example implementations, the processors 504 or processor-implemented components may be distributed across a number of geographic locations.

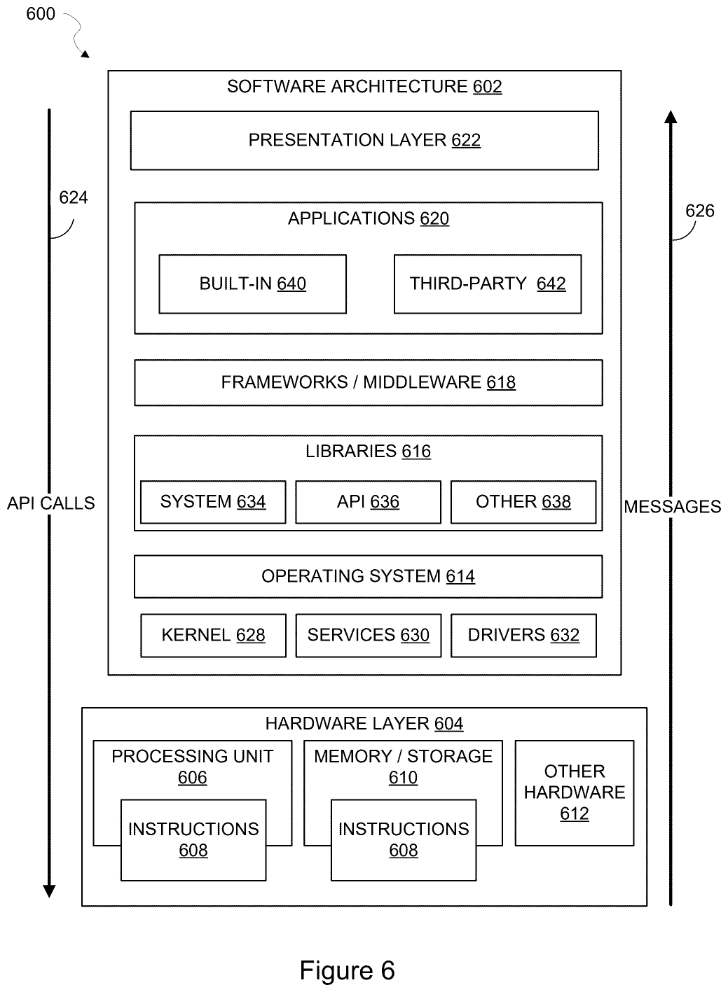

FIG. 6 is a block diagram illustrating system 600 that includes an example software architecture 602, which may be used in conjunction with various hardware architectures herein described. FIG. 6 is a non-limiting example of a software architecture, and it will be appreciated that many other architectures may be implemented to facilitate the functionality described herein. The software architecture 602 may execute on hardware such as machine 500 of FIG. 5 that includes, among other things, processors 504, memory/storage 506, and input/output (I/O) components 508. A representative hardware layer 604 is illustrated and can represent, for example, the machine 500 of FIG. 5. The representative hardware layer 604 includes a processing unit 606 having associated executable instructions 608. Executable instructions 608 represent the executable instructions of the software architecture 602, including implementation of the methods, components, and so forth described herein. The hardware layer 604 also includes at least one of memory or storage modules memory/storage 610, which also have executable instructions 608. The hardware layer 604 may also comprise other hardware 612.

In the example architecture of FIG. 6, the software architecture 602 may be conceptualized as a stack of layers where each layer provides particular functionality. For example, the software architecture 602 may include layers such as an operating system 614, libraries 616, frameworks/middleware 618, applications 620, and a presentation layer 622. Operationally, the applications 620 or other components within the layers may invoke API calls 624 through the software stack and receive messages 626 in response to the API calls 624. The layers illustrated are representative in nature and not all software architectures have all layers. For example, some mobile or special purpose operating systems may not provide a frameworks/middleware 618, while others may provide such a layer. Other software architectures may include additional or different layers.

The operating system 614 may manage hardware resources and provide common services. The operating system 614 may include, for example, a kernel 628, services 630, and drivers 632. The kernel 628 may act as an abstraction layer between the hardware and the other software layers. For example, the kernel 628 may be responsible for memory management, processor management (e.g., scheduling), component management, networking, security settings, and so on. The services 630 may provide other common services for the other software layers. The drivers 632 are responsible for controlling or interfacing with the underlying hardware. For instance, the drivers 632 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 616 provide a common infrastructure that is used by at least one of the applications 620, other components, or layers. The libraries 616 provide functionality that allows other software components to perform tasks in an easier fashion than to interface directly with the underlying operating system 614 functionality (e.g., kernel 628, services 630, drivers 632). The libraries 616 may include system libraries 634 (e.g., C standard library) that may provide functions such as memory allocation functions, string manipulation functions, mathematical functions, and the like. In addition, the libraries 616 may include API libraries 636 such as media libraries (e.g., libraries to support presentation and manipulation of various media format such as MPEG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 616 may also include a wide variety of other libraries 638 to provide many other APIs to the applications 620 and other software components/modules.

The frameworks/middleware 618 (also sometimes referred to as middleware) provide a higher-level common infrastructure that may be used by the applications 620 or other software components/modules. For example, the frameworks/middleware 618 may provide various graphical user interface functions, high-level resource management, high-level location services, and so forth. The frameworks/middleware 618 may provide a broad spectrum of other APIs that may be utilized by the applications 620 or other software components/modules, some of which may be specific to a particular operating system 614 or platform.

The applications 620 include built-in applications 640 and third-party applications 642. Examples of representative built-in applications 640 may include, but are not limited to, a contacts application, a browser application, a book reader application, a location application, a media application, a messaging application, or a game application. Third-party applications 642 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 642 may invoke the API calls 624 provided by the mobile operating system (such as operating system 614) to facilitate functionality described herein.

The applications 620 may use built-in operating system functions (e.g., kernel 628, services 630, drivers 632), libraries 616, and frameworks/middleware 618 to create Uls to interact with users of the system. Alternatively, or additionally, in some systems, interactions with a user may occur through a presentation layer, such as presentation layer 622. In these systems, the application/component “logic” can be separated from the aspects of the application/component that interact with a user.

At least some of the processes described herein can be embodied in computer-readable instructions for execution by one or more processors such that the operations of the processes may be performed in part or in whole by the functional components of one or more computer systems. Accordingly, computer-implemented processes described herein are by way of example with reference thereto, in some situations. However, in other implementations, at least some of the operations of the computer-implemented processes described herein can be deployed on various other hardware configurations. The computer-implemented processes described herein are therefore not intended to be limited to the systems and configurations described with respect to FIGS. 5 and 6 and can be implemented in whole, or in part, by one or more additional system and/or components.

Although the flowcharts described herein can show operations as a sequential process, many of the operations can be performed in parallel or concurrently. In addition, the order of the operations can be re-arranged. A process is terminated when its operations are completed. A process can correspond to a method, a procedure, an algorithm, etc. The operations of methods may be performed in whole or in part, can be performed in conjunction with some or all of the operations in other methods, and can be performed by any number of different systems, such as the systems described herein, or any portion thereof, such as a processor included in any of the systems.

### EXAMPLES

Example 1. In this analysis, we demonstrated our highly sensitive targeted assay that simultaneously captures both genomic alterations and methylation signatures in cell-free DNA (cfDNA). Our assay can detect differential methylations that classify cancer from healthy donors, as well as the quantification of promoter methylation. To capture tumor-associated methylated cfDNA signals, we developed a custom assay on a broad genomic panel (˜15.2 Mb) targeting unmethylated regions in plasma cfDNA from healthy individuals. This panel covers promoter regions of 11,950 genes, including well-known tumor suppressor genes (TSGs) e.g., PTEN, TP53 and homologous recombination and repair (HRR) genes, e.g., BRCA1, RAD51. The analysis of the sequencing data to generate quantitative metrics/measures used in this example was performed using one or more techniques described herein.

For each sample that runs through the assay, with the pre-defined promoter regions of each covered gene, a methylation score is derived from observed methylation signatures. To train the method of making promoter hyper-methylation calls, we first trained and evaluated the specificity of the model on blood samples of 131 cancer-free donor. We then tested the performance on a validation dataset of blood samples from 559 cancer patients (203 lung cancer, 146 breast cancer, 151 bladder cancer, 32 CRC and 27 other cancer types) and 2,631 cancer-free donors.

In blood samples of these 131 cancer-free donors, we established the threshold of making promoter-methylation calls. We focus our analysis on 88 cancer-related genes.

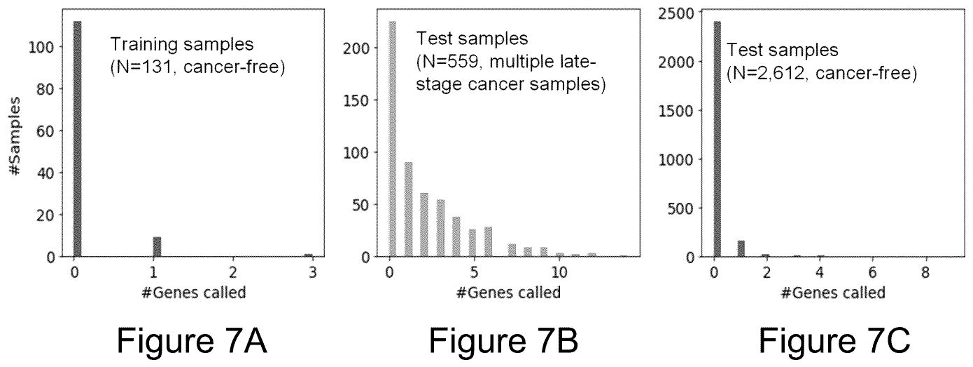

Under our established threshold, 12 promoter methylations (in at least one of the 88 genes) was observed across all 131 training samples (FIG. 7A). In the test dataset of 2,612 cancer-free donors, we observed a total of 251 promoter methylation calls in these 88 genes (FIG. 7C)—from that the specificity is estimated >99%. In the test data of 559 late-stage cancer patients, 334 (60%) were called with at least one promoter methylation in these genes. With samples that get promoter methylation calls, the median calls per samples is 3 (FIG. 7B).

We manually examined the distribution of methylation signals in cancer patients and healthy donors from the test data. In cancer patients with methylation calls, we observed signals mostly within CpG clusters. In most cancer-free donors, we observed no signals associated with methylation in this promoter region: e.g., in MLH1 promoter region, we didn't observe any methylation signals in the 131 healthy-donors from the training set.

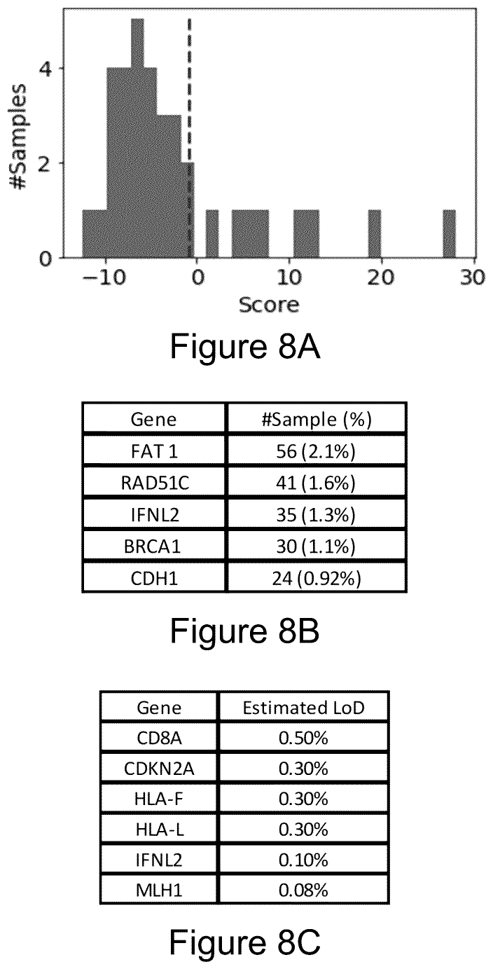

To further investigate the supposed false positives (FP), we examined cancer-prediction scores for cancer-free that have more than one methylation call. We found 40% have high cancer-prediction scores (>10 times of standard deviation than the cancer-normal cutoff, FIG. 8A). As “cancer-free” donors are self-identified in this cohort, it is likely these two donors have already developed cancer. FIG. 8B is a table showing genest that are called most often for promoter methylation in cancer-free donors.

For samples with low cancer-prediction scores, we also found likely true methylations for a few cancer-related genes, e.g. BRCA1. Publications indicate that promoter methylations may happen sporadically in the general population at a very low rate.

We calculated LoD of our method by in-silico mixing KM12 cell line and cancer-free donors at the level of 0.1%, 0.3%, 0.5%, 0.7% and 1%. We calculated LoD for 6 of the cancer-related genes that was called as promoter hyper-methylation with high signals in the cell line KM12 (FIG. 8C).

For each gene, its LoD is affected by methylation levels and its background noise in the healthy population. MLH1 with a fully methylation in KM12 (validated by orthogonal experiments) and has a very low methylation rate in the general population, has the lowest LoD among all genes. For the other 5 genes, their LOD ranges between 0.1% and 0.5%.

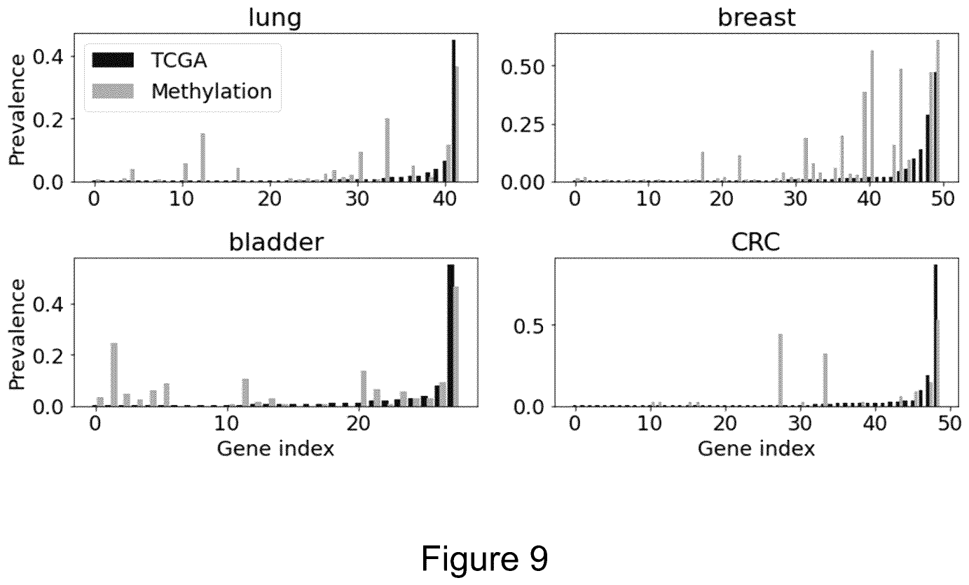

To further validate the promoter hyper-methylation and to validate our findings, we examined the TSG and HRR genes in cancer samples from the TCGA database. We focus our analysis on 2,380 primary tumor cancer samples with available methylation data from 450K array. For each gene, we calculated its prevalence (percentage of samples observed with promoter methylation) in both TCGA data and our test dataset (FIG. 9).

For most genes we observed similar prevalence between TCGA and our methylation detection method. It is worthwhile to note that samples in TCGA are tissue samples and many of them are still in early-stage, but our test dataset is mostly cfDNA from blood of late-stage patients.

We additionally examined the two genes (CD8A and CDKN2A) with high prevalence in our CRC samples but not in TCGA CRC data. The CD8A promoter region was covered by five array probes and two of them show >0.5 prevalence but this high prevalence was lowered by the other three array probes that show no methylation signals. A similar situation happens with CDKN2A which has 1 out of 5 array probes showing a prevalence of 0.1 but the other 4 have very low level of methylation signals.

To showcase the feasibility of our assay, we further summarized promoter methylation in MLH1. Previous studies have shown that 54%-100% of CRC patients with micro-satellite instability (MSI-H) tumor harbor MLH1 promoter methylation.

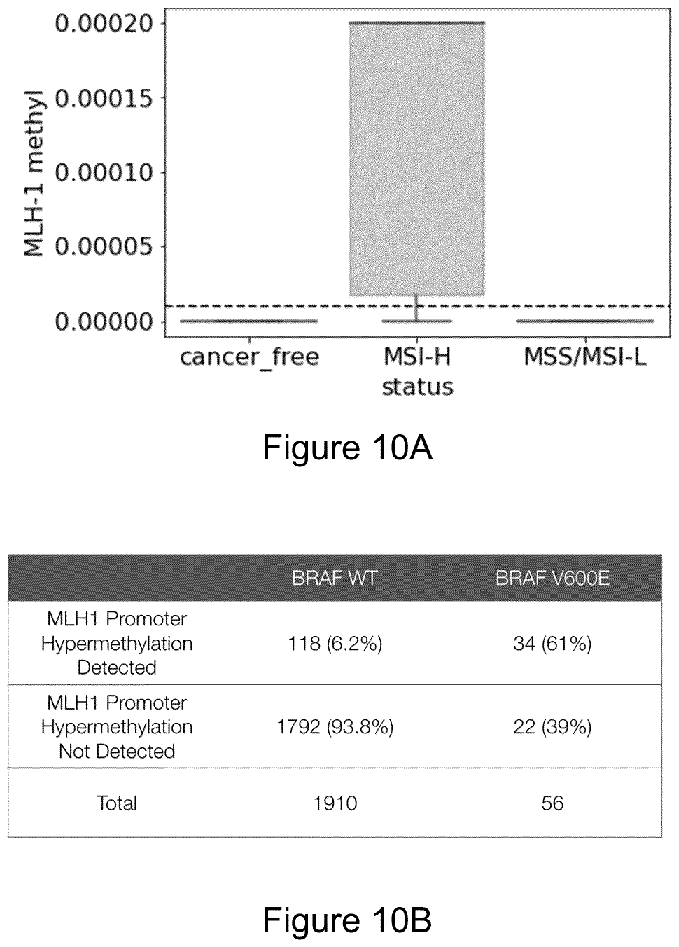

In 1,966 CRC patients, we detected significantly higher MLH1 promoter methylation in MSI-H group (Fisher's p<0.05), compared to patients with micro-satellite stable (MSS) status and the cancer-free population (FIG. 10A).

Furthermore, we tested the prevalence of the mutation, BRAF-V600E, a common driver mutation for CRC. Our finding shows the strong co-occurrence of MLH1 promoter hyper-methylation and BRAF-V600E (FIG. 10B), which is consistent with previous findings.
Example 2. Liquid biopsy offers a rapid and non-invasive alternative to tissue biopsy for identifying biomarkers. More recently, its application has broadened to include assessment of early response to therapy (i.e. molecular response) and in the early-stage settings, detection of minimal residual disease (MRD) and early disease recurrence¹. While circulating tumor fraction (cTF) estimated by somatic mutations is well associated with the tumor progression and prognosis, interference can occur from clonal hematopoiesis of indeterminate potential (CHIP), and for cell-free DNA (cfDNA) samples that lack detectable somatic mutations, somatic tumor fraction cannot be estimated. In this analysis, we demonstrate that epigenomic signatures accurately measure cTF using orthogonal analytes to somatic mutations and enable CTF estimation even in cases without detectable tumor driver variants.

To capture tumor-associated methylated cfDNA, we designed a custom assay on a broad genomic panel (15.2 Mb) that targets unmethylated regions in plasma cfDNA from healthy individuals. DNA molecules that support methylation were enriched by our assay and this information was post-processed into our machine learning model.

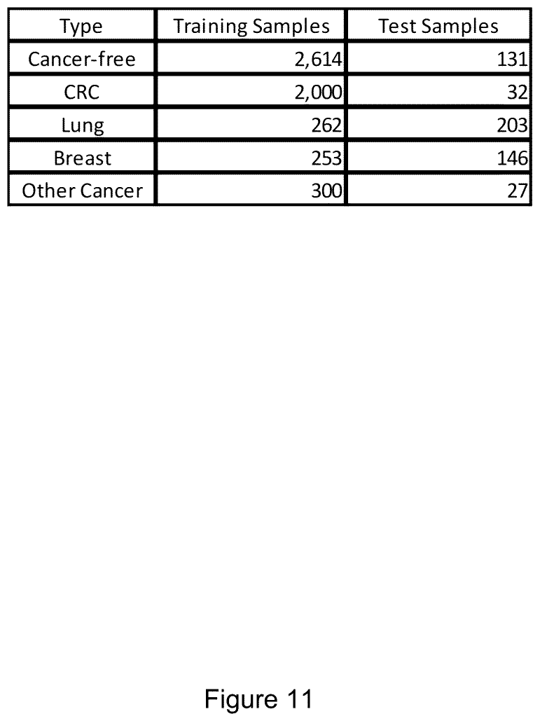

With this panel, we profiled plasma samples from a training set of ˜2,000 cancer patients and ˜2,600 cancer-free donors (FIG. 11). For the prediction of cancer/cancer-free status, we trained a logistic regression model. For tumor-fraction prediction, we trained a linear model utilizing the allele frequency of genomic variant calls as the underlying truth. The training performance was validated by 5-fold cross validation.

On the test dataset, we profiled 559 cancer patients 131 cancer-free donors. We applied all cancer-specific prediction models onto the test dataset to estimate 1) the cancer/cancer-free classification performance of single models and aggregated model; 2) the tumor fraction prediction performance.

To further benchmark the accuracy of our methylation models, we built an in-vitro and an in-silico titration datasets. The in-vitro titration dataset was generated by mixing cfDNA from patients with colorectal cancer (CRC) into the plasma from cancer-free donors via experimental titration. The in-silico titration dataset was generated by computationally mixing sequencing reads from CRC patients with those from cancer-free donors.

CTFs from methylated cfDNA may overcome the current limitations of somatic mutation-based methods. We demonstrate that our methylation approach is capable of accurately detecting cTFs in tumor-driver positive and negative cases. Our assay can reliably enrich molecules with methylation signals in differentially methylated signals in cancer patients. Our cancer-specific models achieves >90% detection rate for late-stage cancer patients while maintaining a 95% specificity. With genomic driver mutation calls as the surrogate for truth tumor fraction, our methylation-based prediction has a correlation of 0.85 with this surrogate on CRC.

As we estimate tumor-negative cases to be 30-50% of patients with stage I-III cancer and 15-20% of patients with stage IV cancer, our methylation approach may hold promise for providing better evaluation for patient care and management.

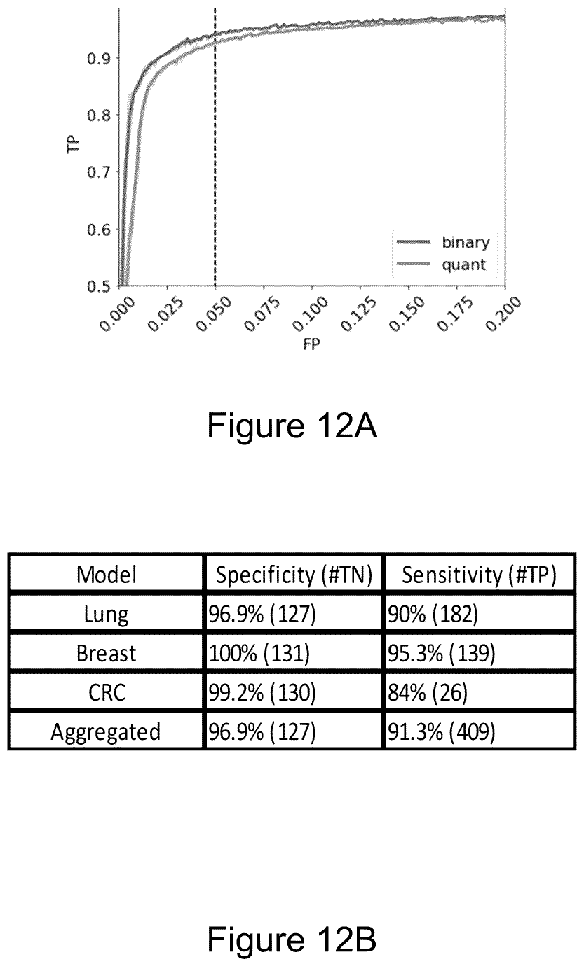

We built our machine-learning models for cancer/cancer-free classification and tumor-fraction prediction on the training dataset of 2,000 cancer patients and 2,614 cancer-free donors. We first evaluated our methods on the training dataset via five-fold cross validation—this process was repeated for 10 times. At 95% specificity, our prediction model for cancer/cancer-free status has an average of 93% detection rate for samples across all stages. The tumor-fraction prediction model has a similar performance as the status prediction model (FIG. 12A). The analysis of the sequencing data to generate quantitative metrics/measures used in this example was performed using one or more techniques described herein.

After the training process, we applied the trained models and their 95% specificity cutoff to the independent test dataset of 559 cancer patients and 131 cancer-free donors. On the test dataset, we observed a 97% specificity with a total of 4 false positives (FPs) observed across all three models. The FP in CRC model is included in the 4 FPs in the lung model (FIG. 12B).

The test dataset is all late-stage patients, the sensitivity of the breast cancer model (95%) is higher than the all-stage sensitivity (62%) in the training dataset where most (>70%) of its breast cancer patients are at early stage.

Manual examinations revealed that the FPs are slightly above the tumor-normal cutoff, as well as that some strong signals come from regions with sporadic mutations in cancer-free donors. It is possible that with advanced region definition and fine-tuning of the models, the FPs can be eliminated. On the other hand, a few FNs have relatively weaker signals in differentially methylated regions, indicating these samples might be less represented in the training data and addition of extra samples may help to increase the detection power.

We applied our CRC tumor-fraction prediction model onto the in-silico titration dataset of 1,000 samples, generated by randomly mixing reads from 1,000 CRC patient samples with 1,000 cancer-free donors. The method quantified a cTF over 0.1% in >99% of these samples. In contrast, when applied to 2,037 cancer-free donors, <5% of the samples resulted in an estimated cTFs >0.1%.

We applied our CRC tumor-fraction prediction model along with our genomic caller for CRC driver mutations on an in-vitro dataset comprised of 270 samples that were generated by experimentally titrating plasma from CRC patients into cancer-free donors at different levels.

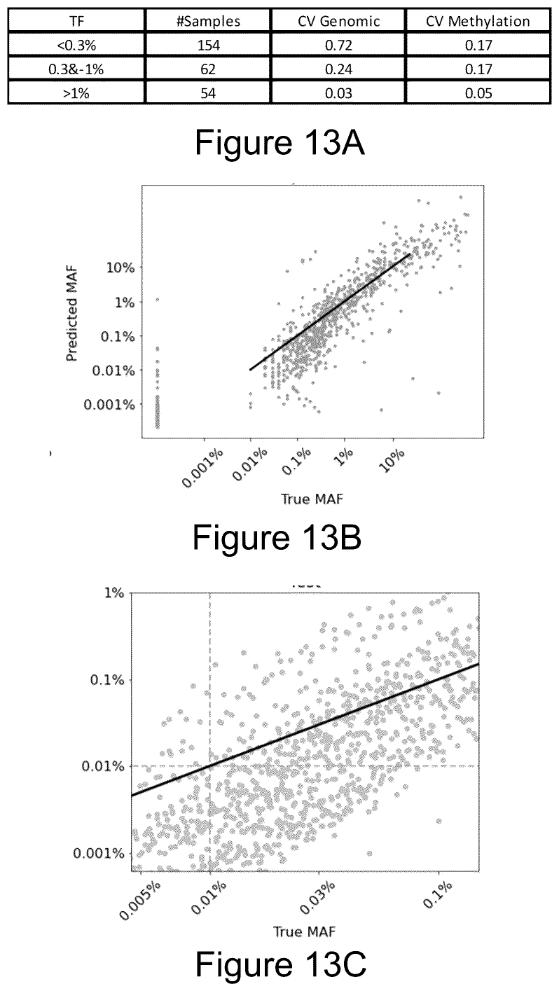

In all samples at low TF, the coefficient of variation (CV) of methylation was more robust than the CV from CTF estimated by genomic mutations: at TF of 0.3˜1%, methylation-based TF has a 5-fold lower than genomic TF (FIG. 13A).

With the predefined set of driver genes for CRC from previous studies, we used the allele frequency of driver mutations called from genomic data as the approximation for underlying true tumor fraction. We first compared the predicted tumor fraction from cross-validation against the true tumor fraction in the training set (FIG. 13B). With a Pearson correlation of 0.85, most of the methylation-based tumor fraction are consistent with the underlying truth.

For the top 10 outliers that have significantly higher methylation-predicted TF than the truth TF, 6 are cancer-free samples with high similarity to CRCs across our entire comprehensive panel. For the 19 outliers significantly lower methylation-predicted TF than the truth TF, it is possible that their extremely high genomic TF (>10%) comes from CHIP that escaped our driver-mutation filters.

It is possible that methylation can resolve samples with TF<0.1%; however, this possibility is difficult to evaluate as cTFx<0.1% are below the estimated limit of detection (LoD) for most of current companion diagnostic products. To test this out, we in-silico titrated molecules from 100 CRC patients into molecules from 100 cancer-free donors at the level between 0.005% and 0.01% and applied our trained models to this dataset (FIG. 13C).

While the cTFs of simulated samples in the 0.03%˜0.01% range were consistent with the methylation-based cTF, estimates broke down below 0.01%. We hypothesize that below this level, there aren't enough true methylation signals existing above noise signals to allow for a robust cTF prediction.

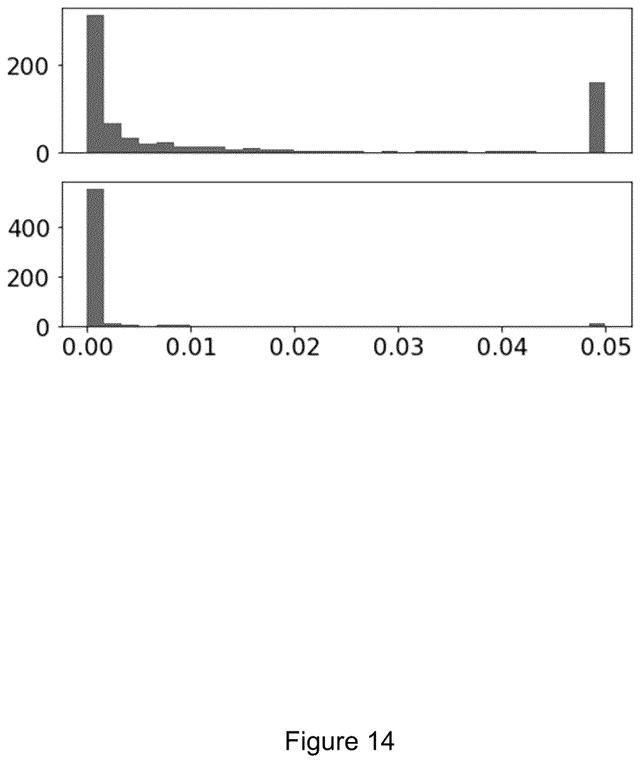

Previous studies have shown that 30-50% of patients with stage I-III cancer, and 15-20% of patients with stage IV cancer, may lack detectable driver mutations. Current methods relying on driver mutations thus cannot quantify the tumor fraction for these patients, leaving a large population with unmet need. Here, we demonstrated that methylation-based cTFs correlate strongly with mutation-based cTFs, thereby enabling the prediction of tumor fraction for this 15-50% of patients (FIG. 14).

As expected, the median predicted TF in these patients is lower than those with detectable driver mutations (0.3% vs 0.02%).
Example 3. Multi-cancer blood-based tests may yield clinical benefit in two ways: by improving adherence to guideline recommended cancer screening with a more convenient, easier to administer, and patient friendly modality, and by detection of early (stage I/II) tumors in cancer types that lack screening recommendations, yet early detection and intervention can save lives. A single test with clinically meaningful performance which addresses both opportunities has yet to be developed.

We evaluated a cfDNA device based on CpG methylation analysis that enables detection of colorectal (CRC), lung cancer, and multiple additional solid tumor cancer types with specificity thresholds tailored by cancer class based on current screening recommendations and clinical diagnostic pathways. The analysis of the sequencing data to generate quantitative metrics used in this example was performed using one or more techniques described herein.

Methods. Blood samples were obtained from multiple cohorts of individuals with colorectal (N>2,000), lung (N>300), and other solid tumor cancers (bladder, gastric, liver, ovarian, pancreas (N>300)) as well as individuals without cancer (N>3,000).

A liquid biopsy CRC screening test, which integrates genomics, epigenomics, and proteomics for the detection of cancer, is the backbone of the device. Lung and multi-cancer detection algorithms were developed through further analysis of the epigenomics patterns of the plasma derived cfDNA, which is enriched in the assay for fragments with high CpG density and high degree of methylation. The analysis of the sequencing data to generate quantitative metrics/measures used in this example was performed using one or more techniques described herein.

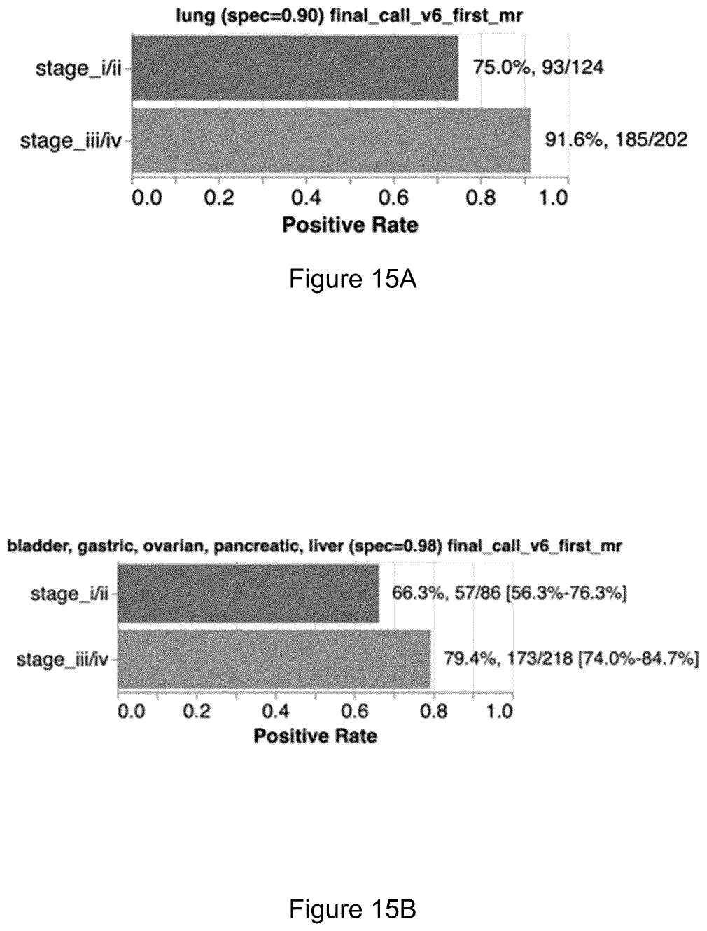

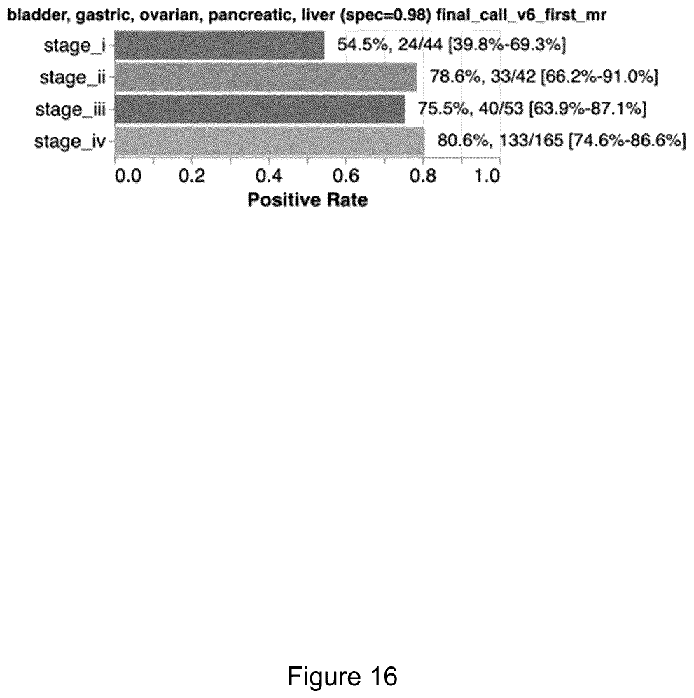

Sensitivity for CRC and lung cancer detection is calculated at 90% target specificity thresholds. Multi-cancer specificity targeted a 98% threshold. Lung cancer and multi-cancer detection performances are obtained through combining the cross validation results from the development set and the results from a single pre-locked model on the validation set. FIG. 15A is a graphical representation showing positivity rates in individual for lung cancer detection in stage I/II patients and in page III/IV. patients. FIG. 15B is a graphical representation showing positivity rates in individuals for multi-cancer detection (bladder, gastric, ovarian, pancreatic, and liver) in stage I/II patients and in stage III/IV patients. Additionally, FIG. 16 is a graphical representation showing a more granular breakdown of positivity rates in individuals for multi-cancer detection (bladder, gastric, ovarian, pancreatic, and liver) in stage I patients, stage II patients, stage III patients, and stage IV patients. The specificity thresholds for CRC, lung, and multi-cancer are selected to yield assay performance tailored for the cancer type and clinical diagnostic pathway.

Results. This single device with tailored thresholds yielded CRC sensitivity of 91% (stage I/II: 93%) and lung cancer sensitivity of 85% (stage I/II: 75%) at 90% specificity. Multi-cancer sensitivity was 75% (stage I/II: 66%) at 98% specificity.

Conclusions. This blood-based cancer screening and detection device, with performance tailored to current cancer screening recommendations and clinical diagnostic pathways, yields performance on par with currently available screening tests for cancers with screening guidelines (CRC and lung) and clinically meaningful early-stage detection in cancer types without screening guidelines where early intervention can bring clinical benefit. With this profile, this multi-cancer blood-based test could cover 32% of the expected cancer diagnoses in 2022 according to SEER estimates, with 80% overall sensitivity (stage I/II: 78%), highlighting the ability of this technology to yield clinically meaningful results for the detection of early stage cancer.
Example 4. Circulating tumor DNA (ctDNA) level and the change in level at a subsequent time point (e.g., on-treatment change from baseline or postoperative changes through time) are promising tools for predicting patient prognosis and response to therapy. Existing methods commonly use minor allele frequency (MAF) of somatic mutations to quantify circulating tumor fraction (cTF). Their performance can be limited by the number of detectable somatic mutations and the associated limit of detection (LoD), as well as interference from copy number variation and non-tumor alterations, such as clonal hematopoiesis (CHIP). Here, we describe the LoD, precision and limit of quantitation (LoQ) of cTF level and change using a next generation sequencing panel covering over 800 genes with genome-wide methylation detection. The analysis of the sequencing data to generate quantitative metrics used in this example was performed using one or more techniques described herein.

The epigenomics cTF (represented by epiMAF) of a single sample is estimated from methylation signals across targeted regions of the methylation panel, calibrated using our internal training data that has clinical blood draw samples of over 5,000 individuals, including cancer-free donors and patients with mixed cancer types. Epigenomics cTF change compares two or more samples from the same patient to identify patient-specific methylated regions, and compare the methylation signals of the paired regions. Somatic mutations also were detected through the genomic panel

LoD was determined as the minimum cTF level at which >=95% of replicates exhibited methylation signals derived from tumors. LoQ was defined as the minimum cTF level at which the coefficient of variation (CV) across replicates was less than 30%. The accuracy of methylation-based cTF was compared to cTFs derived from the maximum MAF of somatic mutations on 5,045 clinical samples of cancer patients.

One colorectal cancer sample, one breast cancer sample, one lung cancer sample, and one cell line sample were titrated into cancer-free backgrounds at target levels ranging from 0.1% to 0.5% MAF. The methylation LoD, which was defined as the lowest concentration of tumor-derived DNA detectable with >95% accuracy, was estimated to be approximately 0.05%.

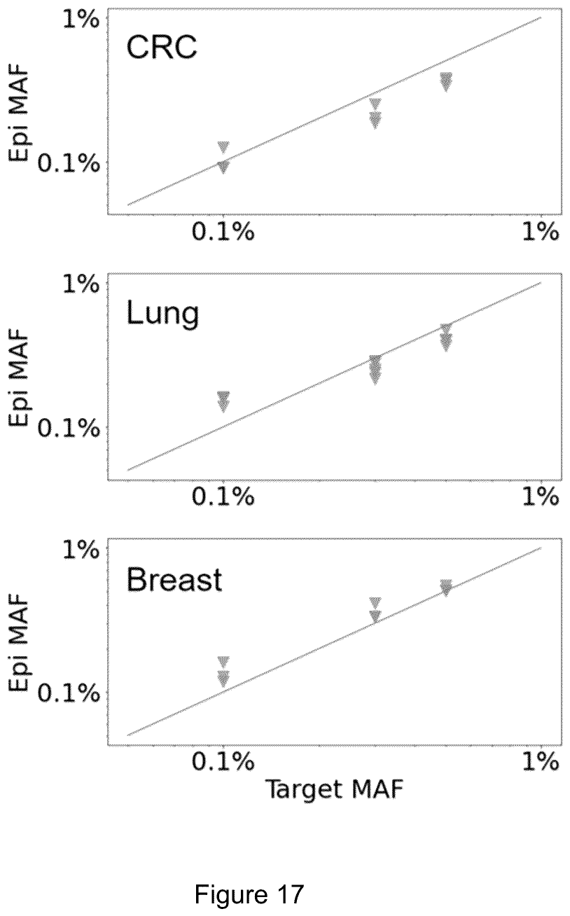

FIG. 17 is a graphical representation of epigenomic MAF in relation to target MAF for colorectal cancer, lung cancer, and breast cancer and indicates the accuracy of epigenomic cTF in clinical filtrations. The epigenomics CTF of clinical samples exhibit a high degree of consistency with underlying titration levels and maintain a strong linearity between different titration levels, as indicated by a Pearson-r of greater than 0.9 and a linearity error less than 5%.

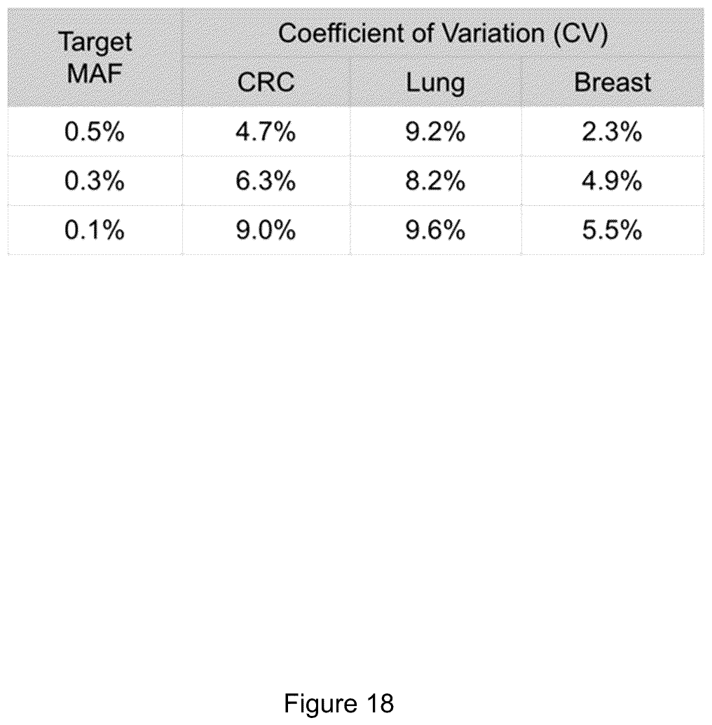

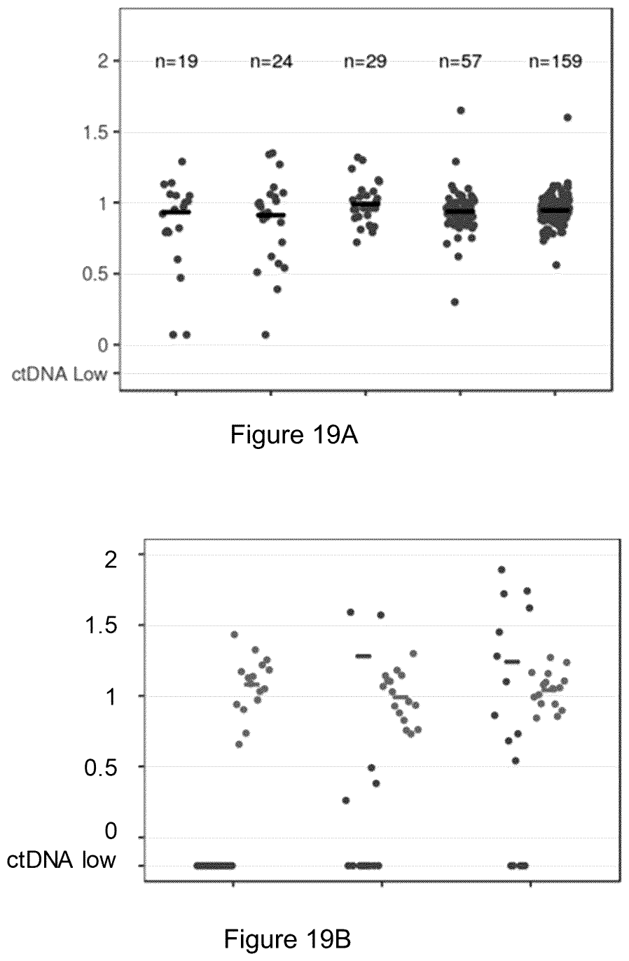

FIG. 18 is a table showing epigenomic cTF variations in technical replicates and indicates that the quantitative precision of epigenomics cTF is capable of reaching an LoQ of less than 0.1% in CRC, lung and breast clinical samples. FIG. 19A is a graphical representation showing that the somatic mutation based cTF is robust for replicates within the same cTF levels, particularly at cTF levels of 0.5% or higher. However, at lower titration levels, the epigenomic cTF is more stable. FIG. 19B is a graphical representation showing that the epigenomic cTF can maintain a 100% evaluation rate and has a LoQ down to 0.1% cTF.

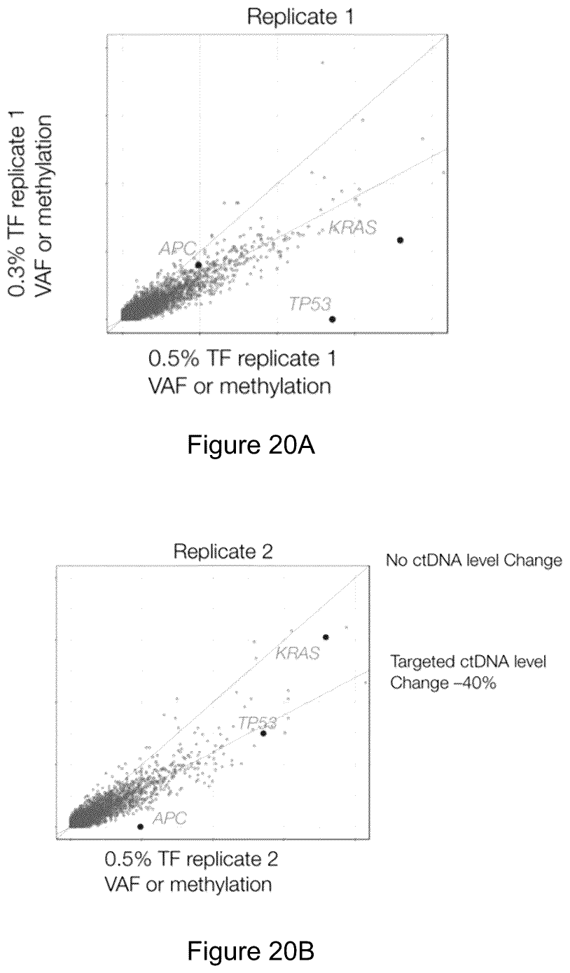

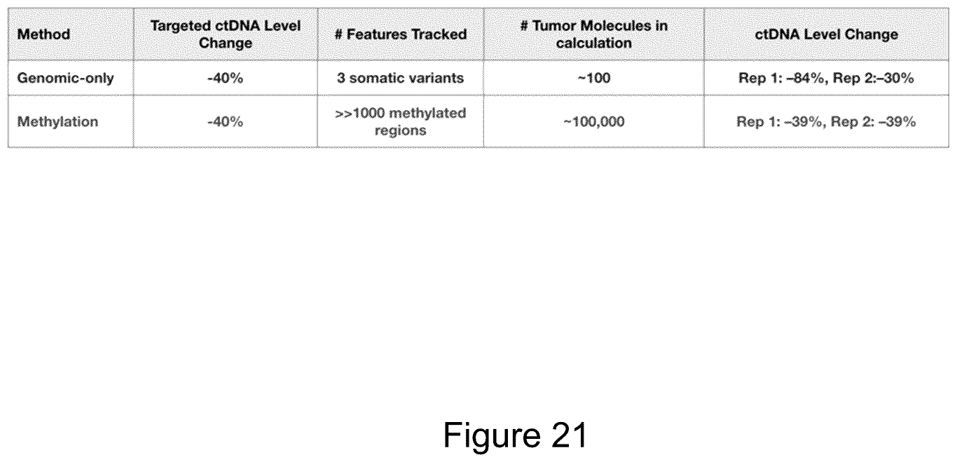

The robustness of epiMAF at low CTF is attributed to the high number of “evaluable” regions in the panel. Specifically, in two technical replicates of a clinical colorectal cancer (CRC) sample with titration levels at 0.5% and 0.3% cTF, epiMAF was estimated based on thousands of regions, whereas genomic cTF can only be estimated from three detectable somatic mutations. FIG. 20A is a graphical representation of methylation signals and somatic mutations for a first replicate of clinical titrations. FIG. 20B is a graphical representation of methylation signals and somatic mutations for a second replicate of clinical titrations. FIG. 21 is a table indicating ctDNA level changes for the first replicate and the second replicate calculated using a genomic-only method and a methylation method.

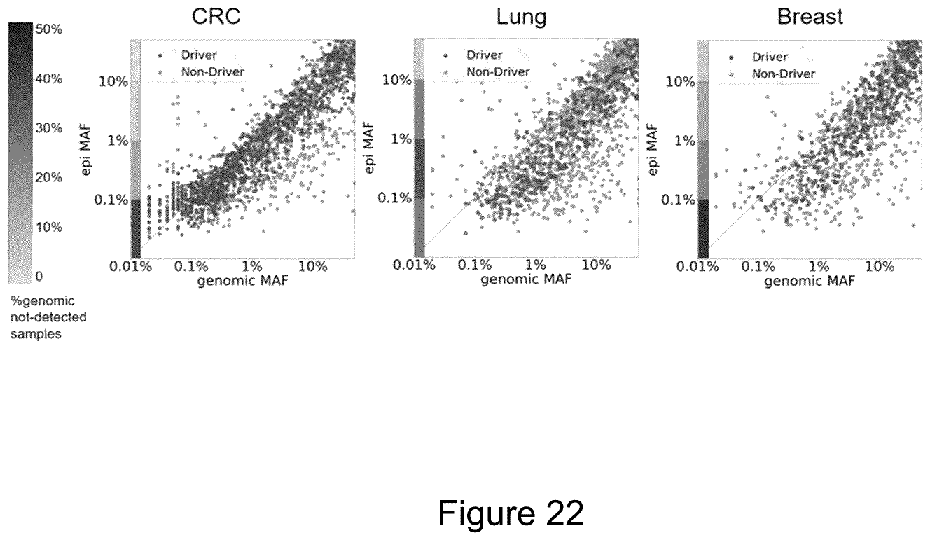

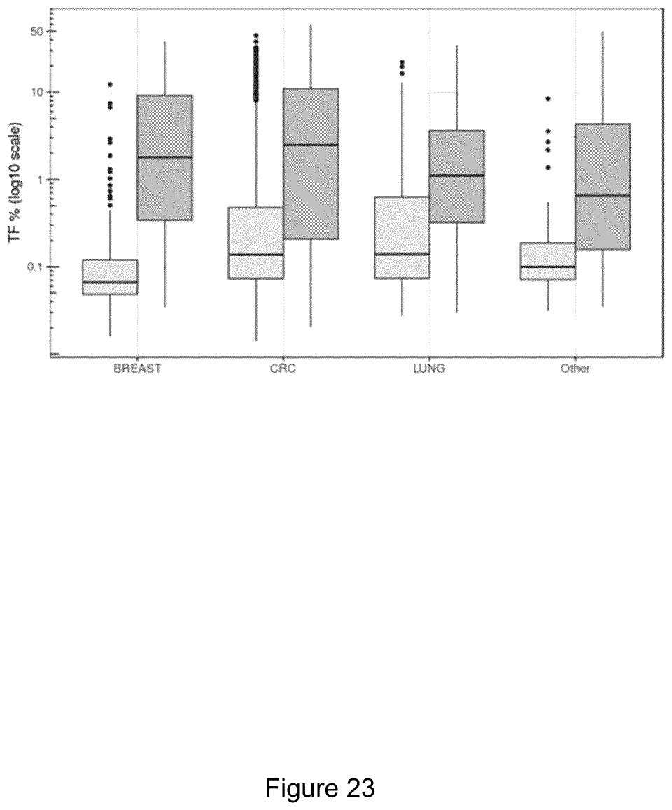

In a cohort of 5,045 clinical samples (CRC, lung, and breast cancer patients, (N=522, 909, 696 and 784 for stage I to IV, together with 2,656 of unknown stage), 64% had somatic mutations, and 90% showed evidence of the ctDNA presence based on methylation analysis. Notably, the epiMAF method produces highly consistent estimates with somatic MAFs for patients with “driver” somatic mutations, which are likely to be a more accurate representation of the true cTF. FIG. 22 is a graphical representation of epigenomic vs genomic cTF on clinical samples (one point for one sample). FIG. 23 is a graphical representation of the epiMAF distribution in early and late stage cancer patients for breast cancer, colorectal cancer, lung cancer, and a group of other cancers.

Upon analyzing 231 additional samples from various cancer types, we found that the majority of samples lacking detectable somatic mutations had tumor fractions below 0.1% (as shown in the side bars of FIG. 22). Patients with early-stage cancer are more prone to have such low tumor fractions where the current somatic mutation-based methods are unable to detect or quantify the mutations as indicated in FIG. 23.

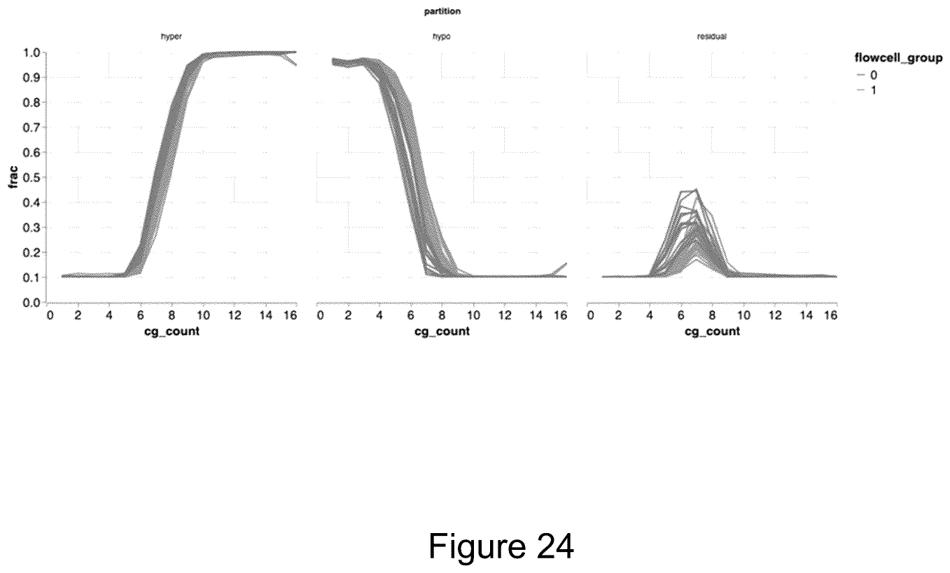

Methylome sequencing enables accurate quantification of ctDNA level with a liquid-only approach, offering easy-to-access longitudinal ctDNA monitoring. Previous studies show that 30-50% patients with stage I-III cancer, and 15-20% patients with stage IV cancer, lack detectable somatic mutations. The methodologies described herein accurately detect and quantify cTF in these patients, improving patient evaluations and disease management.
Example 5. A number of subjects provided samples that were analyzed according to a cell-free DNA assay. The analysis of the sequencing data to generate quantitative metrics used in this example was performed using one or more techniques described herein. Information derived from the samples by treating the samples with a number of solutions including MBD to separate molecules of the samples into a first partition, a second partition, and a third partition with each partition representing a different amount of methylated cytosines in genomic regions having an amount of CG content. The molecules having a given number of methylated CpGs were determined for each partition. FIG. 24 is a graphical representation showing a probability distribution indicating the number of methylated cytosines included in the three partitions.

For each sample, an MBD partitioning profile was calculated as the number of methylated molecules from positive control regions with a given number of CpG divided by the total number of methylated molecules from positive control regions with CpG count above a certain threshold. The ratios or the logarithmic transformation of the ratios for CpG count in certain range are used to revise the region level methylation measurement. The adjusted measurement equals to the unadjusted measurement subtracting the MBD partitioning offset. The offset is estimated as the weighted sum of the ratios or the logarithmic transformation of the ratios. In some example, the CpG threshold is set at 12, 13, 14 and 15, and the CpG ranges are 1-30 CpGs, 4-8 CpGs and 6-12 CpGs.

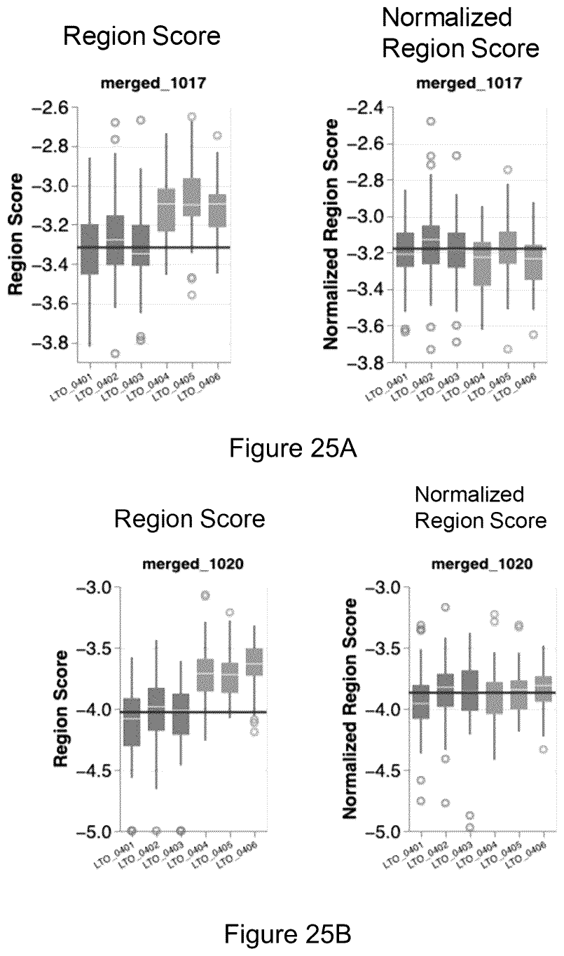

FIG. 25A includes a graphical representation showing changes to region level methylation measurements for a first classification region for a first group of samples treated with MBD using a first set of reagents and a second group of samples treated with MBD using a second set of reagents. The region score indicates unadjusted region level methylation measurements prior to applying the normalization method while the normalized region score indicates the adjusted region level methylation measurement. Additionally, FIG. 25B includes a graphical representation showing changes to region level methylation measurements for a second classification region for a first classification region for a first group of samples treated with MBD using a first set of reagents and a second group of samples treated with MBD using a second set of reagents. The region score indicates unadjusted region level methylation measurements prior to applying the normalization method while the normalized region score indicates the adjusted region level methylation measurement.

1.-60. (canceled)

61. A method comprising:
obtaining, by a computing system having one or more hardware processors and memory, testing data from an individual, the testing data including individual sequence representations having a threshold amount of methylated cytosines included in one or more portions of the individual sequence representations having at least threshold cytosine-guanine content;
analyzing, by the computing system, the testing data to determine first counts of a first number of the individual sequence representations that correspond to individual classification regions of a plurality of classification regions that have the threshold amount of methylated cytosines and that have at least the threshold cytosine-guanine content;
analyzing, by the computing system, the testing data to determine second counts of a second number of the individual sequence representations that correspond to individual control regions of a plurality of control regions;
determining, by the computing system, a metric for the individual classification regions based on a ratio of (i) the first counts for the individual classification regions and (ii) the second counts for the individual control regions;
generating, by the computing system, an input vector that includes the metrics for the individual classification regions; and
determining, by the computing system, an indication of a biological condition being present in the individual by providing the input vector to a model that implements one or more machine learning techniques, the model including weights for individual classification regions of the plurality of classification regions and at least a portion of the weights of the individual classification regions being different from one another.

62. The method of claim 61, comprising:
obtaining, by the computing system having one or more hardware processors and memory, training data including additional sequence representations having a threshold amount of methylated cytosines included in one or more portions of individual additional sequence representations having at least a threshold cytosine-guanine content;
analyzing, by the computing system, the training data to determine additional first counts of a first number of the additional sequence representations that corresponds to individual classification regions of the plurality of classification regions;
analyzing, by the computing system, the training data to determine an additional second counts of a second number of the additional sequence representations that correspond to individual control regions of a plurality of control regions;
determining, by the computing system, an additional metric for the individual classification regions based on an additional ratio of (i) the additional first counts for the individual classification regions and (ii) the additional second counts for the individual control regions;
generating, by the computing system, additional training data that includes the additional metric for the individual classification regions; and
implementing, by the computing system and using the additional training data, the one or more machine learning techniques to generate the model to determine indications of the biological condition being present in individuals.

63. The method of claim 61, wherein:
the one or more machine learning techniques include one or more classification algorithms; and
the indication of the biological condition corresponds to a first numerical indicator of the biological condition being present in the individual.

64. The method of claim 61, wherein the one or more machine learning techniques include one or more regression algorithms; and
the indication of the biological condition corresponds to a second numerical indicator of the biological condition being present in the individual.

65. The method of claim 61, wherein the one or more machine learning techniques include:
a classification algorithm that determines a first numerical indication of the biological condition being present in the individual; and
a regression algorithm that determines a second numerical indication of the biological condition being present in the individual;
wherein an integration system combines the first numerical indication and the second numerical indication to determine an aggregated numerical indication of the biological condition being present in the individual.

66. The method of claim 61, wherein the metric for the individual classification regions is determined based on the ratio of the first counts for the individual classification regions and the second counts for the individual control regions, a scaling factor and an error correction factor.

67. The method of claim 62, comprising:
performing, by the computing system, a training process using the training data and the additional training data to generate the model, wherein the training process includes:
determining, by the computing system, one or more additional weights of individual samples used to produce the training data based on the indication of the biological condition for the individual samples being within a threshold confidence level.

68. The method of claim 67, wherein the indication of the biological condition for an individual sample is outside of the threshold confidence level and the method comprises:
applying, by the computing system, a penalty to a weight of the individual sample during the training process.

69. The method of claim 67, comprising:
performing, by the computing system and using the one or more machine learning techniques, one or more first iterations of the training process for the model using a portion of the training data; and
generating, by the computing system, first output data for the model based on the one or more first iterations of the training process, the first output data corresponding to one or more first additional indications of the biological condition being present in first individuals, wherein first samples obtained from the first individuals are used to produce the portion of the training data.

70. The method of claim 69, comprising:
combining, by the computing system, the first output data and the training data to produce further training data;
performing, by the computing system, one or more second iterations of the training process for the model using a portion of the further training data; and
generating, by the computing system, second output data for the model based on the one or more second iterations of the training process, the second output data indicating one or more second additional indications of the biological condition being present in second individuals, wherein second samples obtained from the second individuals are used to produce the portion of the additional training data;
wherein the weights for the individual classification regions of the plurality of classification regions are determined based on the first output data and the second output data.

71. The method of claim 70, wherein the indication of a biological condition being present in the individual includes at least one of a tumor fraction estimate, an indication that circulating tumor DNA is detected in a sample obtained from the individual, an indication that circulating tumor DNA is not detected in the sample obtained from the individual, a probability of a tumor being present in the individual, or an indication of one or more tissues from which cancer cells present in the individual originate.

72. The method of claim 67, comprising:
determining, by the computing system, that a number of indications of the biological condition being present that were determined during one or more iterations of the training process are at least a threshold value; and
determining, by the computing system, that modifications to one or more weights of the model are not modified or are modified by a minimal amount.

73. The method of claim 72, comprising:
determining, by the computing system, that an additional number of indications of the biological condition being present that were determined during the one or more iterations of the training process are less than the threshold value; and
determining, by the computing system, that modifications to one or more additional weights of the model are modified by more than the minimal amount.

74. The method of claim 61, wherein a limit of detection for the model to determine the indication of the biological condition is no greater than 0.05%.

75. A computing system includes:
one or more hardware processors; and
one or more non-transitory computer-readable storage media including computer-readable instructions that, when executed by the one or more hardware processors, cause the one or more hardware processors to perform operations comprising:
obtaining testing data from an individual, the testing data including individual sequence representations having a threshold amount of methylated cytosines included in one or more portions of the individual sequence representations having at least threshold cytosine-guanine content;
analyzing the testing data to determine first counts of a first number of the individual sequence representations that correspond to individual classification regions of a plurality of classification regions that have the threshold amount of methylated cytosines and that have at least the threshold cytosine-guanine content;
analyzing the testing data to determine second counts of a second number of the individual sequence representations that correspond to individual control regions of a plurality of control regions;
determining a metric for the individual classification regions based on a ratio of (i) the first counts of the individual classification regions and the second counts of the individual control regions;
generating an input vector that includes the metrics for the individual classification regions; and
determining an indication of a biological condition being present in the individual by providing the input vector to a model that implements one or more machine learning techniques, the model including weights for individual classification regions of the plurality of classification regions and at least a portion of the weights of the individual classification regions being different from one another.

76. The computing system of claim 75, wherein the one or more non-transitory computer-readable storage media include additional computer-readable instructions that, when executed by the one or more hardware processors, cause the one or more hardware processors to perform additional operations comprising:
determining, using the testing data, a distribution of the individual sequence representations for a differentially methylated region;
determining that at least a threshold amount of the individual sequence representations included in the distribution overlap with a subregion of the differentially methylated region; and
determining that the subregion of the differentially methylated region is a classification region of the plurality of classification regions.

77. The computing system of claim 76, wherein the threshold amount of sequence representations is at least about 70% of the sequence representations included in the distribution.

78. The computing system of claim 75, wherein:
the one or more non-transitory computer-readable storage media include additional computer-readable instructions that, when executed by the one or more hardware processors, cause the one or more hardware processors to perform additional operations comprising:
determining an order of values of the metrics; and
determining a subset of the individual classification regions from among the plurality of classification regions based on the order; and
a portion of the metrics that correspond to the subset of the individual classification regions is used to determine the indication of the biological condition being present in the individual.

79. The computing system of claim 75, wherein:
the indication of the biological condition being present in the individual is an initial indication of the biological condition being present in the individual; and
the one or more non-transitory computer-readable storage media include additional computer-readable instructions that, when executed by the one or more hardware processors, cause the one or more hardware processors to perform additional operations comprising:
applying a scaling factor to the initial indication of the biological condition being present in the individual to determine a modified indication of the biological condition being present in the individual.

80. One or more non-transitory computer-readable storage media including computer-readable instructions that, when executed by one or more processors, cause the one or more processors to perform operations comprising:
obtaining testing data from an individual, the testing data including individual sequence representations having a threshold amount of methylated cytosines included in one or more portions of the individual sequence representations having at least threshold cytosine-guanine content;
analyzing the testing data to determine first counts of a first number of the individual sequence representations that correspond to individual classification regions of a plurality of classification regions that have the threshold amount of methylated cytosines and that have at least the threshold cytosine-guanine content;
analyzing the testing data to determine second counts of a second number of the individual sequence representations that correspond to individual control regions of a plurality of control regions;
determining a metric for the individual classification regions based on a ratio of (i) the first counts for the individual classification regions and (ii) the second counts for the individual control regions;
generating an input vector that includes the metrics for the individual classification regions; and
determining an indication of a biological condition being present in the individual by providing the input vector to a model that implements one or more machine learning techniques, the model including weights for individual classification regions of the plurality of classification regions and at least a portion of the weights of the individual classification regions being different from one another.
